(19) United States
(12) Patent Application Publication
(10) Pub. No.: US 2024/0174745 A1
(43) Pub. Date: May 30, 2024

(54) Antibody Constructs for CLDN18.2 and CD3

(71) Applicants: AMGEN RESEARCH (MUNICH) GMBH, Munich (DE); AMGEN INC., Thousand Oaks, CA (US)

(72) Inventors: Christoph Dahlhoff, Munich (DE); Claudia Blümel, Munich (DE); Johannes Brozy, Munich (DE); Tobias Raum, Munich (DE); Elisabeth Nahrwold, Munich (DE); Tara Arvedson, Thousand Oaks, CA (US); Irwin Chen, Thousand Oaks, CA (US); Sandra Ross, Thousand Oaks, CA (US); Julie Bailis, Thousand Oaks, CA (US)

(21) Appl. No.: 18/316,739

(22) Filed: May 12, 2023

Related U.S. Application Data
(63) Continuation of application No. 16/530,006, filed on Aug. 2, 2019, now Pat. No. 11,692,031.

(51) Int. Cl. C07K 16/28; A61K 39/00; A61P 35/00

(52) U.S. Cl. CPC C07K 16/28; A61P 35/00; C07K 16/2809; A61K 2039/505; C07K 2317/31; C07K 2317/33; C07K 2317/565; C07K 2317/622; C07K 2317/73; C07K 2317/92; C07K 2317/94

(57) ABSTRACT

The present invention relates to an antibody construct comprising a domain which binds to Claudin 18.2 (CLDN18.2) and another domain which binds to CD3. Moreover, the invention provides a polynucleotide encoding the antibody construct, a vector comprising said polynucleotide and a host cell transformed or transfected with said polynucleotide or vector. Furthermore, the invention provides a process for producing the antibody construct of the invention, a medical use of said antibody construct and a kit comprising said antibody construct.

This application includes a sequence listing submitted electronically by the file name 53378B_Seqlisting.XML; Size: 380,143 bytes; Created: May 11, 2023, and is incorporated herein by reference.

The present invention relates to an antibody construct comprising a domain which binds to Claudin 18.2 (CLDN18.2) and another domain which binds to CD3. Moreover, the invention provides a polynucleotide encoding the antibody construct, a vector comprising said polynucleotide and a host cell transformed or transfected with said polynucleotide or vector. Furthermore, the invention provides a process for producing the antibody construct of the invention, a medical use of said antibody construct and a kit comprising said antibody construct.

### BACKGROUND OF THE INVENTION

Claudins are key structural and functional components of epithelial tight junctions, which act to regulate cell-cell permeability, maintain ion homeostasis, and support cell adhesion and polarity. Claudins are tetraspan transmembrane proteins of 22-27 kDa that multimerize within or across cell membranes to form a protective barrier. The 24 claudin proteins that have been reported differ by the specificity of their tissue localization and by their interactions with other proteins.

Claudin 18 (CLDN18) was initially identified as a target gene for the transcription factor T/EBP/NKX2.1. Consistent with its homology to other claudin family members, CLDN18 was confirmed to localize to cellular tight junctions in mouse and human. CLDN18 was shown to encode two isoforms generated by alternative splicing: CLDN18.1, expressed specifically in normal lung, and CLDN18.2, expressed in differentiated cells of the gastric mucosa.

CLDN18.2 is a 261 amino acid protein with two extracellular loops, and has 92% sequence identity to CLDN18.1. Unlike the second extracellular loop, the first extracellular loop of CLDN18.2 has eight amino acid differences from CLDN18.1. CLDN18.2 homology to other family members is more limited, with 29-34% overall identity to CLDN1, CLDN6 and CLDN7.

CLDN18.2 is expressed in several tumor types, including gastric cancer, pancreatic cancer, esophageal cancer, mucinous ovarian cancer and non-small cell lung cancer. CLDN18.2 expression in gastric cancer includes the invasive front and metastatic sites, although absolute levels of CLDN18 are reported to be decreased in these settings. The expression of CLDN18.2 in multiple tumor types, with normal tissue expression mainly restricted to differentiated cells in the stomach, has led to the consideration of CLDN18.2 as a therapeutic target in gastric cancer and other indications.

Gastric and gastroesophageal cancers remain indications with high unmet medical need, with at least 1.4 million new cases and 1.1 million deaths reported annually worldwide (Lordick and Janjigian, Nat Rev Cancer 2016). Typical first-line treatment involves surgery and combination chemotherapy that includes platinum and fluoropyrimidine compounds. Although this regimen can potentially improve quality of life and extend survival 8-10 months on median, 5-year survival rates remain low.

Targeted therapies offer an alternative strategy for treatment. The anti-Her2 monoclonal antibody trastuzumab is approved for first-line treatment of Her2-positive gastric and gastroesophageal cancer, in combination with chemotherapy (Bang et al., The Lancet 2010). The anti-VEGFR2 antibody ramucirumab is approved for treatment of gastric and gastroesophageal cancer that has progressed after chemotherapy (Fuchs et al., The Lancet 2014). While these targeted agents further increase survival compared to chemotherapy alone, their efficacy has been limited by the heterogeneity of target expression and by resistance mechanisms.

More recently, immune checkpoint therapy has demonstrated activity in select settings: pembrolizumab was approved in the United States for treatment of microsatellite instability-high (MSI-H) tumors, including gastric cancer (Le et al., Science 2017), and for treatment of unresectable advanced or recurrent gastric cancer that progressed after two or more lines of chemotherapy (Fuchs et al., J Clin Oncol 2017). Nivolumab was approved in Japan for treatment of unresectable advanced or recurrent gastric cancer that progressed after chemotherapy (Kang et al., The Lancet, 2017). Only 1-2% of patients in unselected populations, and 60% of the MSI-H (microsatellite instability high) population (9% of total gastric and gastroesophageal cases) demonstrated a complete response in these studies. New therapies with the potential to provide durable response to a larger patient population are therefore still needed.

Pancreatic cancer has proven to be even less responsive to available therapy than gastric or gastroesphageal cancers. There are at least 338,000 cases and 331,000 deaths from pancreatic cancer reported worldwide annually, with a median survival of 6 months (Ilic and Ilic, World J Gastroenterol 2016). Just 20-30% of patients are candidates for surgical resection. Gemcitabine has been considered first-line therapy until recently, when the combination chemotherapy regimens FOLFIRINOX (5-fluorouracil, leucovorin, oxaliplatin, irinotecan), and gemcitabine with nab-paclitaxel, were shown to increase overall survival ˜2-5 months over gemcitabine treatment alone (Uccello et al., Curr Oncol 2018). Other chemotherapy combinations are typically used in second-line therapy. Several targeted therapy and immunotherapy agents have been evaluated in advanced pancreatic cancer with limited success. Pancreatic tumors are characterized by desmoplasia and immunosuppressive immune infiltrates which drives this lack of response. New therapies with the potential to overcome this immunosuppressive environment and extend survival are needed.

Bispecific antibody constructs comprising one domain that binds to CD3 on a T cell and one domain that binds to a protein expressed on a target cell directly connect T cells to target cells to induce T cell redirected lysis. This mechanism of action is distinct from chemotherapy, targeted therapy and other immunotherapy in that it can work with any CD3-positive T cell, independent of a costimulatory activating signal (Klinger et al., Immunol Reviews 2016). The expression of CLDN18.2 on the cell surface of gastric cancer, gastroesophageal cancer and pancreatic cancer provides a basis for targeting these tumor types with a CLDN18.2×CD3 antibody construct. Furthermore, a CLDN18.2×CD3 antibody construct has the potential to target additional tumor types that express CLDN18.2, including mucinous ovarian cancer, colorectal cancer and non-small cell lung cancer.

### DETAILED DESCRIPTION OF THE INVENTION

Thus, in one aspect, the present invention provides an antibody construct comprising a first domain which binds to CLDN18.2 on the surface of a target cell and a second domain which binds to CD3 on the surface of a T cell.

It is also envisaged that:
(1) the first domain of the antibody construct of the invention binds to the first extracellular loop (loop 1) of CLDN18.2;
(2) the first domain of the antibody construct of the invention binds to the same epitope of CLDN18.2 as an antibody or antibody construct comprising a domain which binds to CLDN18.2 on the surface of a target cell and which comprises:
   a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
   b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
   c) a VH region as depicted in SEQ ID NO: 127, and a VL region as depicted in SEQ ID NO: 128; or
   d) a VH region as depicted in SEQ ID NO: 139, and a VL region as depicted in SEQ ID NO: 140;
(3) the antibody construct of the invention competes for binding with an antibody or antibody construct comprising a domain which binds to CLDN18.2 on the surface of a target cell and which comprises:
   a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
   b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
   c) a VH region as depicted in SEQ ID NO: 127, and a VL region as depicted in SEQ ID NO: 128; or
   d) a VH region as depicted in SEQ ID NO: 139, and a VL region as depicted in SEQ ID NO: 140;
(4) the first domain of the antibody construct of the invention binds to a CLDN18.2 mutant on the surface of a target cell having the amino acid sequence as depicted in SEQ ID NO: 22, and optionally also binds to a CLDN18.2 mutant on the surface of a target cell having the amino acid sequence as depicted in SEQ ID NO: 24, but does not bind to a CLDN18.2 mutant on the surface of a target cell having the amino acid sequence as depicted in SEQ ID NO: 23;
(5) the first domain of the antibody construct of the invention binds to a CLDN18.2 mutant on the surface of a target cell having the amino acid sequence as depicted in SEQ ID NO: 14 and/or to a CLDN18.2 mutant on the surface of a target cell having the amino acid sequence SEQ ID NO 15, and optionally also binds to one or more CLDN18.2 mutants on the surface of a target cell having an amino acid sequence selected from the group consisting of those depicted in SEQ ID NOs: 11, 12, 13, 16, 17, 19, 20 and 21, but does not bind to a CLDN18.2 mutant on the surface of a target cell having the amino acid sequence as depicted in SEQ ID NO: 18;
(6) the first domain of the antibody construct of the invention binds to human CLDN18.2 on the surface of a target cell, wherein Glu (E) at position 56 of human CLDN18.2 is essential for the binding of the first domain, and Ala (A) at position 42 and/or Asn (N) at position 45 of human CLDN18.2 is/are not essential for the binding of the first domain; and/or
(7) the first domain of the antibody construct of the invention binds to an epitope of CLDN18.2 comprising the amino acid sequence as depicted in SEQ ID NO: 266, but not comprising the amino acid sequence as depicted in SEQ ID NO: 265, and optionally also not comprising the amino acid sequence as depicted in SEQ ID NO: 267.

Advantageously, targeting the epitope of CLDN18.2 which is recognized by the antibody constructs of the present invention (see also Example 2) provides the following benefits:
(1) selectivity of the CLDN18.2×CD3 antibody constructs over CLDN18.1 (see Example 6), and
(2) an unexpectedly high cytotoxic potency for the CLDN18.2×CD3 antibody constructs (see Example 7.4).

The term “antibody construct” refers to a molecule in which the structure and/or function is/are based on the structure and/or function of an antibody, e.g., of a full-length immunoglobulin molecule. An antibody construct hence immunospecifically binds to its target or antigen, and/or it comprises the heavy chain variable region (VH) and/or the light chain variable region (VL) of an antibody, or comprises domains derived therefrom. An antibody construct according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. An antibody construct may hence be characterized by the presence of three or six CDRs in either one or both binding domains, and the skilled person knows where (in which order) those CDRs are located within the binding domain.

The definition of “antibody” according to the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Antibody constructs” of the present invention may have the structure of a full-length immunoglobulin as it occurs naturally. For example, they may comprise (at least) two full-length antibody heavy chains and two full-length antibody light chains. However, given that the antibody constructs according to the invention comprise one domain binding to CLDN18.2 and another domain binding to CD3, they do not occur naturally, and they are markedly different in their function from naturally occurring products. An antibody construct of the invention is hence an artificial “hybrid” molecule comprising at least two distinct binding domains with different specificities.

“Antibody constructs” of the present invention may also comprise fragments of full-length antibodies, such as VH, VHH, VL, (s)dAb, Fv, light chain (VL-CL), Fd (VH-CH1), heavy chain, Fab, Fab′, F(ab′)2 or “r IgG” (“half antibody” consisting of a heavy chain and a light chain). Antibody constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL, that specifically binds to an antigen or target independently of other variable regions or domains. Further possible formats of the antibody constructs according to the invention are cross bodies, maxi bodies, hetero Fc constructs, mono Fc constructs and scFc constructs. Examples for those formats will be described herein below.

Furthermore, the definition of the term “antibody construct” includes bivalent and polyvalent/multivalent constructs as well as bispecific and polyspecific/multispecific constructs, which specifically bind to two, three or more antigenic structures, through distinct binding domains. An antibody construct can have more binding valences than specificities, e.g. in a case where it has two binding domains for the first target (Cldn18.2) and one binding domain for the second target (CD3), or vice versa, in which case the construct is trivalent and bispecific. In general, the term “bispecific” includes the meaning that an antibody construct binds to (at least) two different antigens, such as Cldn18.2 and CD3.

Moreover, the definition of the term “antibody construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and antibody constructs derived therefrom are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009.

The term “binding domain” or “domain which binds to . . . ” characterizes in connection with the present invention a domain of the antibody construct which immunospecifically binds to/interacts with/recognizes an epitope on the target or antigen (here: CLDN18.2 in the case of the first domain, and CD3 in the case of the second domain). The structure and function of the first domain (binding to CLDN18.2), and preferably also the structure and/or function of the second domain (binding to CD3), is/are based on the structure and/or function of an antibody, e.g. of a full-length immunoglobulin molecule. The “binding domain” or “domain which binds to . . . ” may hence comprise the minimum structural requirements of an antibody which allow for immunospecific target binding. This minimum structural requirement of the first domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. It is envisaged that the second domain also comprises this minimum structural requirement of an antibody which allow for the immunospecific target binding. More preferably, the second domain also comprises at least three light chain CDRs (i.e. CDR1, CDR2 and CDR3 of the VL region) and/or three heavy chain CDRs (i.e. CDR1, CDR2 and CDR3 of the VH region), preferably all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain.

Examples for the format of a “domain which binds to” (or a “binding domain”) include, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′), or “r IgG” (“half antibody”)), antibody variants or derivatives such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” (selected from formats such as (VH-VL-CH3)₂. (scFv-CH3)₂, ((scFv)₂-CH3+CH3)), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)2); (3) an antibody fragment or variant comprising VH and CH1 (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of antibody constructs or binding domains according to the invention are e.g. described in WO 00/006605, WO 2005/040220, WO 2008/119567, WO 2010/037838, WO 2013/026837, WO 2013/026833, US 2014/0308285, US 2014/0302037, W O2014/144722, WO 2014/151910, and WO 2015/048272.

It is envisaged for the antibody construct of the present invention that
a) the antibody construct is a single chain polypeptide or a single chain antibody construct, b) the first domain is in the format of an scFv,
c) the second domain is in the format of an scFv,
d) the first and the second domain are connected via a linker, preferably a peptide linker, more preferably a glycine/serine linker, and/or
e) the antibody construct comprises a domain providing an extended serum half-life, such as an Fc-based domain.

The antibody constructs of the present invention are preferably “in vitro generated antibody constructs” and/or “recombinant antibody constructs”. In the context of the present invention, the term “in vitro generated” refers to an antibody construct according to the above definition where all or part of the binding domain or of a variable region (e.g., at least one CDR) is generated in a non-immune cell selection, e.g., in an in vitro phage display, on a protein chip or in any other method in which candidate amino acid sequences can be tested for their ability to bind to an antigen. This term thus preferably excludes sequences generated solely by genomic rearrangement in an immune cell in an animal. It is envisaged that the first and/or second domain of the antibody construct is produced by or obtainable by phage display or library screening methods rather than by grafting CDR sequences from a pre-existing (monoclonal) antibody into a scaffold. A “recombinant antibody construct” is an antibody construct generated or produced using (inter alia) recombinant DNA technology or genetic engineering.

The antibody constructs of the present invention are envisaged to be monoclonal. As used herein, antibodies or antibody constructs that are denominated “monoclonal” (mAb) are obtained from a population of substantially homogeneous antibodies/antibody constructs, i.e., the individual antibodies/antibody constructs comprised in the population are identical (in particular with respect to their amino acid sequence) except for possible naturally occurring mutations and/or post-translational modifications (e.g., isomerizations, amidations) that may be present in minor amounts. Monoclonal antibodies/antibody constructs are highly specific, being directed against a single epitope within the antigen, in contrast to polyclonal antibody preparations which typically include different antibodies directed against different determinants (or epitopes). In addition to their specificity, monoclonal antibodies are advantageous in that they are synthesized by the hybridoma culture, hence uncontaminated by other immunoglobulins. The modifier “monoclonal” indicates the character of the antibody/antibody construct as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that immunospecifically binds to a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore™ system can be used to increase the efficiency of phage antibodies/antibody constructs which bind to an epitope of a target antigen (Schier, Human Antibodies Hybridomas 7 (1996), 97-105; Malmborg, J. Immunol. Methods 183 (1995), 7-13).

Another exemplary method of making antibody constructs or binding domains includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., Xenomouse™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antibody constructs or binding domains include humanized variants of non-human antibodies/antibody constructs, “affinity matured” antibody constructs or binding domains (see, e.g. Hawkins et al. J. Mol. Biol. 254, 889-896 (1992) and Lowman et al., Biochemistry 30, 10832-10837 (1991)) and antibody variants or mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, antibody constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, antibody constructs or binding domains with affinities in the low nanomolar range.

A preferred type of an amino acid substitutional variation of the antibody constructs or binding domains of the invention involves substituting one or more residues within the hypervariable region of a parent antibody structure (e.g. a humanized or human antibody structure). Generally, the resulting variant(s) selected for further development will have improved biological properties relative to the parent antibody structure from which they are generated. A convenient way for generating such substitutional variants involves affinity maturation using phage display. Briefly, several sites of the hypervariable region (e. g. 6-7 sites) are mutated to generate all possible amino acid substitutions at each site. The variants thus generated are displayed in a monovalent fashion from filamentous phage particles as fusions to the gene III product of M13 packaged within each particle. The phage-displayed variants are then screened for their biological activity (e.g. binding affinity) as disclosed herein. To identify candidate hypervariable region sites contributing significantly to antigen binding (candidates for modification), alanine scanning mutagenesis can also be performed. Alternatively, or additionally, it may be beneficial to analyze a crystal structure of the complex between the antigen and the antibody construct or the binding domain to identify contact points between the binding domain and its specific antigen. Such contact residues and neighbouring residues are candidates for substitution according to the techniques elaborated herein. Once such variants are generated. the panel of variants is subjected to screening as described herein and antibodies, their antigen-binding fragments, antibody constructs or binding domains with superior properties in one or more relevant assays may be selected for further development.

The antibody constructs and binding domains of the present invention specifically include “chimeric” versions in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments or variants of such antibodies, so long as they exhibit the desired biological activity (U.S. Pat. No. 4,816,567; Morrison et al., Proc. Natl. Acad. Sci. USA, 81: 6851-6855 (1984)). Chimeric antibody constructs or binding domains of interest herein include “primitized” antibody constructs comprising variable domain antigen-binding sequences derived from a non-human primate (e.g., Old World Monkey, Ape etc.) and human constant region sequences. A variety of approaches for making chimeric antibodies or antibody constructs have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment, antibody variant or binding domain may also be modified by specific deletion of human T cell epitopes (a method called “deimmunization”) using methods disclosed for example in WO 98/52976 or WO 00/34317. Briefly, the heavy and light chain variable regions of an antibody, antibody construct or binding domain can be analyzed for peptides that bind to MHC class II; these peptides represent potential T cell epitopes (as defined e.g. in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains or variable regions, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory (www2.mrc-lmb.cam.ac.uk/vbase/list2.php) provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, variants or fragments thereof, antibody constructs and binding domains are based on immunoglobulins of mostly human sequences, which contain (a) minimal sequence(s) derived from non-human immunoglobulin. For the most part, humanized antibodies, variants or fragments thereof, antibody constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, antibody constructs and binding domains as used herein may also comprise residues which are found neither in the recipient antibody nor the donor antibody. These modifications are made to further refine and optimize antibody performance. The humanized antibodies, variants or fragments thereof, antibody constructs and binding domains may also comprise at least a portion of an immunoglobulin constant region (such as Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies, variants or fragments thereof, antibody constructs and binding domains can be generated by replacing sequences of the (Fv) variable region that are not directly involved in antigen binding with equivalent sequences from human (Fv) variable regions. Exemplary methods for generating such molecules are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. These methods include isolating, manipulating, and expressing the nucleic acid sequences that encode all or part of immunoglobulin (Fv) variable regions from at least one of a heavy or light chain. Such nucleic acids may be obtained from a hybridoma producing an antibody against a predetermined target, as described above, as well as from other sources. The recombinant DNA encoding the humanized antibody, variant or fragment thereof, antibody construct or binding domain can then be cloned into an appropriate expression vector.

Humanized antibodies, variants or fragments thereof, antibody constructs and binding domains may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized molecules described herein (U.S. Pat. No. 5,225,539). All of the CDRs of a particular human sequence may be replaced with at least a portion of a non-human CDR, or only some of the CDRs may be replaced with non-human CDRs. It is only necessary to replace the number of CDRs required for binding of the humanized molecule to a predetermined antigen.

A humanized antibody, variant or fragment thereof, antibody construct or binding domain can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

Human anti-mouse antibody (HAMA) responses have led the industry to prepare chimeric or otherwise humanized antibodies/antibody constructs. It is however expected that certain human anti-chimeric antibody (HACA) responses will be observed, particularly in chronic or multi-dose utilizations of an antibody or antibody construct. Thus, it would be desirable to provide antibody constructs comprising a human binding domain against CLDN18.2 and/or a human binding domain against CD3, in order to vitiate concerns and/or effects of HAMA or HACA response.

Therefore, according to one embodiment, the antibody construct, the first binding domain and/or the second binding domain are “human”. The term “human antibody”, “human antibody construct” and “human binding domain” includes antibodies, antibody constructs and binding domains, respectively, having antibody-derived regions such as variable and constant regions or domains which correspond substantially to human germline immunoglobulin sequences known in the art, including, for example, those described by Kabat et al. (1991) (loc. cit.). The human antibody constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular in CDR3. The human antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, antibody constructs and binding domains which include only non-artificially and/or genetically altered human sequences of antibodies as those can be derived by using technologies or systems such as the Xenomouse.

Antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. To avoid the use of rodent-derived antibody constructs, humanized or fully human antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs derived therefrom are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse model is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513. Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the antibody constructs of the invention are “isolated” or “substantially pure” antibody constructs. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that could interfere with diagnostic or therapeutic uses for the antibody construct, and may include enzymes, hormones, and other proteinaceous or non-proteinaceous compounds. It is understood that the isolated or substantially pure antibody construct may constitute from 5% to 99.9% by weight of the total protein/polypeptide content in a given sample, depending on the circumstances. The desired antibody construct may be produced at a significantly higher concentration through the use of an inducible promoter or high expression promoter. The definition includes the production of an antibody construct in a wide variety of organisms and/or host cells that are known in the art. In certain embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver staining. Usually, however, an isolated antibody construct will be prepared by at least one purification step.

According to one embodiment, the entire antibody construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins on the basis of size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The terms “(specifically or immunospecifically) binds to”, “(specifically or immunospecifically) recognizes”, or “(specifically or immunospecifically) reacts with” mean in accordance with this invention that an antibody construct or a binding domain interacts or (immuno-)specifically interacts with a given epitope on the target molecule (antigen), here: CLDN18.2 and CD3, respectively. This interaction or association occurs more frequently, more rapidly, with greater duration, with greater affinity, or with some combination of the aforementioned, to an epitope on the specific target than to alternative substances (non-target molecules). Because of the sequence similarity between homologous proteins in different species, an antibody construct or a binding domain that immunspecifically binds to its target (such as a human target) may, however, cross-react with homologous target molecules from different species (such as, from non-human primates). The term “specific/immunospecific binding” can hence include the binding of an antibody construct or binding domain to epitopes or structurally related epitopes in more than one species.

In the context of the present invention, the term “epitope” refers to the part or region of the antigen that is recognized/immunospecifically recognized by the binding domain. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved as a result of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

A method for CLDN18.2 epitope mapping is described in the following: A pre-defined region (a contiguous amino acid stretch) within the extracellular loops of human CLDN18.2 protein is exchanged/replaced with a corresponding region of a CLDN18.2 paralogue (such as human CLDN6 or human CLDN9, but other paralogues are also conceivable, so long as the binding domain is not cross-reactive with the paralogue used). These human CLDN18.2/paralogue chimeras are expressed on the surface of host cells (such as CHO cells). Binding of the antibody or antibody construct can be tested via FACS analysis. When the binding of the antibody or antibody construct to the chimeric molecule is entirely abolished, or when a significant binding decrease is observed, it can be concluded that the region of human CLDN18.2 which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to human (wild-type) CLDN18.2, whereby binding to human CLDN18.2 is set to be 100%. Alternatively, the above described epitope mapping analysis can be modified by introducing one or more point mutations into the sequence of CLDN18.2, specifically the sequence of the extracellular loop 1 or loop 2. These point mutations can e.g. reflect the differences between CLDN18.2 and its closely related paralogue CLDN18.1. For example, the mutations can be selected from the group consisting of: Q29M, N37D, A42S, N45Q, Q47E, E56Q, G65P and L691. See Examples 1 and 2.

A further method to determine the contribution of a specific residue of a target antigen to the recognition by an antibody construct or binding domain is alanine scanning (see e.g. Morrison K L & Weiss G A. Curr Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired.

The interaction between the binding domain and the epitope of the target antigen implies that a binding domain exhibits appreciable or significant affinity for the epitope/the target antigen (here: CLDN18.2 and CD3, respectively) and, generally, does not exhibit significant affinity for proteins or antigens other than the target antigen (here: CLDN18.2/CD3)—notwithstanding the above discussed cross-reactivity with homologous targets e.g. from other species. “Significant affinity” includes binding with an affinity (dissociation constant, KD) of ≤10⁶ M. Preferably, binding is considered specific when the binding affinity is ≤10⁻⁷ M, ≤10⁻⁸ M, ≤10⁻⁹M, ≤10⁻¹⁰ M, or even ≤10⁻¹¹ M, or ≤10⁻¹² M. Whether a binding domain (immuno-)specifically reacts with or binds to a target can be tested readily e.g. by comparing the affinity of said binding domain to its desired target protein or antigen with the affinity of said binding domain to non-target proteins or antigens (here: proteins other than CLDN18.2 or CD3, respectively). Preferably, an antibody construct of the invention does not significantly bind to proteins or antigens other than CLDN18.2 or CD3, respectively (i.e., the first domain does not bind to proteins other than CLDN18.2 and the second domain does not bind to proteins other than CD3)—unless any further binding domain(s) directed against a further target is/are deliberately introduced into the antibody construct of the invention, in which case the binding of that binding domain to its specific target is also provided by the present invention.

It is envisaged that the affinity of the first domain for CLDN18.2 (e.g. human CLDN18.2) is ≤100 nM, ≤90 nM, ≤80 nM, ≤70 nM, ≤60 nM, ≤50 nM, ≤40 nM, ≤30 nM, or ≤20 nM. These values are preferably measured in a cell-based assay, such as a Scatchard assay. See Example 4. Other methods of determining the affinity are also well-known. It is furthermore envisaged that the affinity of the second domain for CD3 (e.g. human CD3) is ≤100 nM, ≤90 nM, ≤80 nM, ≤70 nM, ≤60 nM, ≤50 nM, ≤40 nM, ≤30 nM, ≤20 nM, or ≤10 nM. These values are preferably measured in a surface plasmon resonance assay, such as a Biacore assay. See Example 3.

The term “does not significantly bind” means that an antibody construct or binding domain of the present invention does not bind to a protein or antigen other than CLDN18.2 or CD3, when said protein or antigen is expressed on the surface of a cell. The antibody construct hence shows reactivity of ≤30%, preferably ≤20%, more preferably ≤10%, particularly preferably ≤9%, ≤8%, ≤7%, ≤6%, ≤5%, ≤4%, ≤3%, ≤2%, or ≤1% with proteins or antigens other than CLDN18.2 or CD3 (when said proteins or antigens are expressed on the surface of a cell), whereby binding to CLDN18.2 or CD3, respectively, is set to be 100%. The “reactivity” can e.g. be expressed in an affinity value (see above).

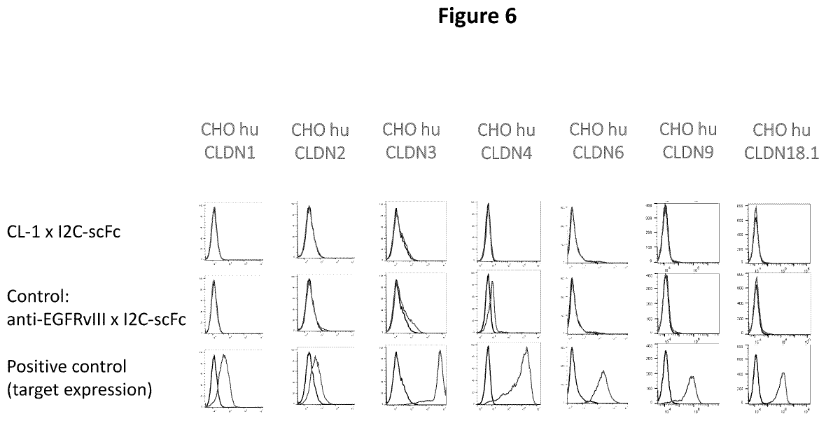

It is envisaged that the antibody construct of the invention (and more specifically its first domain) does not bind or does not significantly bind to CLDN18.2 paralogues, more specifically to human CLDN18.2 paralogues and/or to macaque/cyno CLDN18.2 paralogues. It is also envisaged that the antibody construct does not bind or does not significantly bind to (human or macaque/cyno) CLDN18.2 paralogues on the surface of a target cell. The CLDN18.2 paralogues include—but are not limited to—CLDN18.1, CLDN1, CLDN2, CLDN3, CLDN4, CLDN6, and CLDN9. According to one embodiment, the human paralogues of CLDN18.2 have sequences as depicted in SEQ ID NOs: 2-10. See Example 6 and FIG. 6. It is hence envisaged that the first domain of the antibody construct of the invention does not bind or does not significantly bind to CLDN18.1. CLDN1, CLDN2, CLDN3, CLDN4, CLDN6, and/or CLDN9 (on the surface of a target cell).

The first domain of the antibody construct of the invention binds to CLDN18.2 on the surface of a target cell. The “target cell” can be any prokaryotic or eukaryotic cell expressing CLDN18.2 on its surface; preferably the target cell is a cell that is part of the human or animal body, such as a specific CLDN18.2 expressing cancer or tumor cell or a cell of a CLDN18.2 positive neoplasm. It is understood that the term “on the surface”, in the context of the present invention, means that the first domain of the antibody construct specifically binds to an epitope comprised within the first CLDN18.2 extracellular loop (CLDN18.2 ECL1), within the second CLDN18.2 extracellular loop (CLDN18.2 ECL2), or comprised within a combination of both loops. It is hence envisaged that the first domain of the antibody construct of the invention binds to an extracellular loop of CLDN18.2, preferably of human CLDN18.2. The extracellular loop can be the first loop or the second loop. It is also envisaged that both loops contribute to the binding. In this case, it is possible that one loop (such as the first loop) represents the main binding partner for the antibody construct, and the other loop (such as the second loop) contributes to the binding, e.g. as a stabilizing partner, but is not absolutely essential for the binding. The first domain according to the invention may hence bind to CLDN18.2 when it is expressed by naturally expressing cells or cell lines (such as human gastric cancer lines SNU-601, SNU-620, or also SNU-16, NUGC, NUG-C4, GSU, or IM95), and/or by cells or cell lines transformed or (stably/transiently) transfected with CLDN18.2. In one embodiment, the first domain binds to CLDN18.2 when CLDN18.2 is used as a target molecule in a cell-based binding assay such as Scatchard (see e.g. Example 4). It is furthermore envisaged that the antibody construct/its first domain binds to human CLDN18.2 on the surface of a target cell. A preferred amino acid sequence for human CLDN18.2 is depicted in SEQ ID NO: 1.

It is envisaged that the antibody construct according to the invention (and, more specifically, the first domain of said antibody construct) binds to the first extracellular loop (ECL1, loop 1) of CLDN18.2. This does not necessarily exclude that the second extracellular loop also contributes, albeit to a lesser extent, to the paratope-eptiope interaction site. The term “CLDN18.2 ECL” (ECL=extracellular loop) refers to those parts of CLDN18.2 which are essentially free of the transmembrane and cytoplasmic domains of CLDN18.2. It is understood that the transmembrane domains identified for the CLDN18.2 polypeptide of the present invention are identified pursuant to criteria routinely employed in the art for identifying that type of hydrophobic domain. The exact boundaries of a transmembrane domain may vary but most likely by no more than about 5 amino acids at either end of the domain specifically mentioned herein. A preferred human CLDN18.2 ECL1 is shown in SEQ ID NO: 2, and a preferred human CLDN18.2 ECL2 is shown in SEQ ID NO: 3.

The present invention furthermore provides that:
the first domain of the antibody construct of the invention binds to the same epitope of CLDN18.2 as an antibody or antibody construct comprising a domain which binds to CLDN18.2 on the surface of a target cell and which comprises:
   a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
   b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
   c) a VH region as depicted in SEQ ID NO: 127, and a VL region as depicted in SEQ ID NO: 128; or
   d) a VH region as depicted in SEQ ID NO: 139, and a VL region as depicted in SEQ ID NO: 140;
the antibody construct of the invention competes for binding with an antibody or antibody construct comprising a domain which binds to CLDN18.2 on the surface of a target cell and which comprises:
   a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
   b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
   c) a VH region as depicted in SEQ ID NO: 127, and a VL region as depicted in SEQ ID NO: 128; or
   d) a VH region as depicted in SEQ ID NO: 139, and a VL region as depicted in SEQ ID NO: 140;
the first domain of the antibody construct of the invention binds to a CLDN18.2 mutant on the surface of a target cell having the amino acid sequence as depicted in SEQ ID NO: 22, and optionally also binds to a CLDN18.2 mutant on the surface of a target cell having the amino acid sequence as depicted in SEQ ID NO: 24, but does not bind to a CLDN18.2 mutant on the surface of a target cell having the amino acid sequence as depicted in SEQ ID NO: 23;
the first domain of the antibody construct of the invention binds to a CLDN18.2 mutant on the surface of a target cell having the amino acid sequence as depicted in SEQ ID NO: 14 and/or to a CLDN18.2 mutant on the surface of a target cell having the amino acid sequence SEQ ID NO 15, and optionally also binds to one or more CLDN18.2 mutants on the surface of a target cell having an amino acid sequence selected from the group consisting of those depicted in SEQ ID NOs: 11, 12, 13, 16, 17, 19, 20 and 21, but does not bind to a CLDN18.2 mutant on the surface of a target cell having the amino acid sequence as depicted in SEQ ID NO: 18;
the first domain of the antibody construct of the invention binds to human CLDN18.2 on the surface of a target cell, wherein Glu (E) at position 56 of human CLDN18.2 is essential, and Ala (A) at position 42 and/or Asn (N) at position 45 of human CLDN18.2 is/are not essential for the binding of the first domain; and/or
the first domain of the antibody construct of the invention binds to an epitope of CLDN18.2 comprising the amino acid sequence as depicted in SEQ ID NO: 266, but not comprising the amino acid sequence as depicted in SEQ ID NO: 265, and optionally also not comprising the amino acid sequence as depicted in SEQ ID NO: 267.

Other anti-CLDN18.2 binders (CL-3, CL-4) were also analyzed for their CLDN18.2 binding specificities during epitope mapping (see Example 2). These CLDN18.2×CD3 antibody constructs were found to have a different epitope specificity, and at the same time were shown to have a significantly inferior cytotoxic potential compared with the antibody constructs of the invention. In Example 7.4, it was demonstrated that the antibody constructs of the present invention display EC₅₀ values in the two-digit picomolar range, while the comparative constructs displayed EC₅₀ values in the three-digit up to the five-digit picomolar range, despite having similar affinities for CLDN18.2. Antibody constructs displaying cytotoxic activity of the latter range might not be potent enough for a therapeutic use in directing a patient's immune system, more specifically the T cells' cytotoxic activity, against cancer cells. On the other hand, antibody constructs according to the invention present with a very favorable epitope-activity relationship, hence supporting potent antibody construct mediated cytotoxic activity.

Whether or not an antibody, antibody construct or binding domain binds to the same epitope of CLDN18.2 on the surface of a target cell as another given antibody, antibody construct or binding domain can be measured by different analyses as described herein, e.g. by epitope mapping with chimeric or mutated CLDN18.2 molecules, as described herein above or in Examples 1 and 2. Other methods of determining epitopes are described herein, such as alanine scanning.

Whether or not an antibody or antibody construct competes for binding to an antigen (such as CLDN18.2) on the surface of a target cell with another given antibody or antibody construct can be measured in a competition assay such as a competitive ELISA. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. Preferably a cell-based competition assay is used, using either cells that naturally express CLDN18.2 or cells that were stably or transiently transformed with CLDN18.2. The term “competes for binding”, in the present context, means that competition occurs between the two tested antibodies of at least 20%, at least 30%, at least 40%, at least 50%, at least 60%, at least 70%, at least 80% or at least 90%, as determined by any one of the assays disclosed above, preferably the cell-based assay. The same analysis can of course be applied for other targets such as CD3.

Competitive antibody binding assays include assays determining the competitive binding of two antibodies/antibody constructs to a cell surface bound antigen. Common methods aim to detect binding of two antibodies/antibody constructs, A and B, to the same antigen on the surface of a cell may include steps of:
a) blocking of the cell surface antigen by pre-incubation of cells with antibody/antibody construct A followed by a sub-maximal addition of labeled antibody/antibody construct B and detecting the binding of B compared with binding in the absence of A;
b) titration (i.e. adding different amounts) of antibody/antibody construct A in the presence of sub-maximal amounts of labeled antibody/antibody construct B and detecting the effect on binding of B; or
c) co-titration of A and B, wherein both antibodies/antibody constructs are incubated together at maximal concentration and detecting whether the total binding equals or exceeds that of either A or B alone, i.e. a method which cannot be affected by the order of addition or relative amounts of the antibodies/antibody constructs.

When two antibodies/antibody constructs A and B compete for a cell surface bound antigen, the antibodies will very often compete with each other in blocking assays independently from the order of the addition of the antibodies. In other words, competition is detected if the assay is carried out in either direction. However, this is not always the case, and under certain circumstances the order of the addition of the antibodies or the direction of the assay may have an effect on the signal generated. This may be due to differences in affinities or avidities of the potentially competing antibodies/antibody constructs. If the order of the addition has a significant effect on the signal generated, it is concluded that the two antibodies/antibody constructs do compete if competition is detected in at least one order.

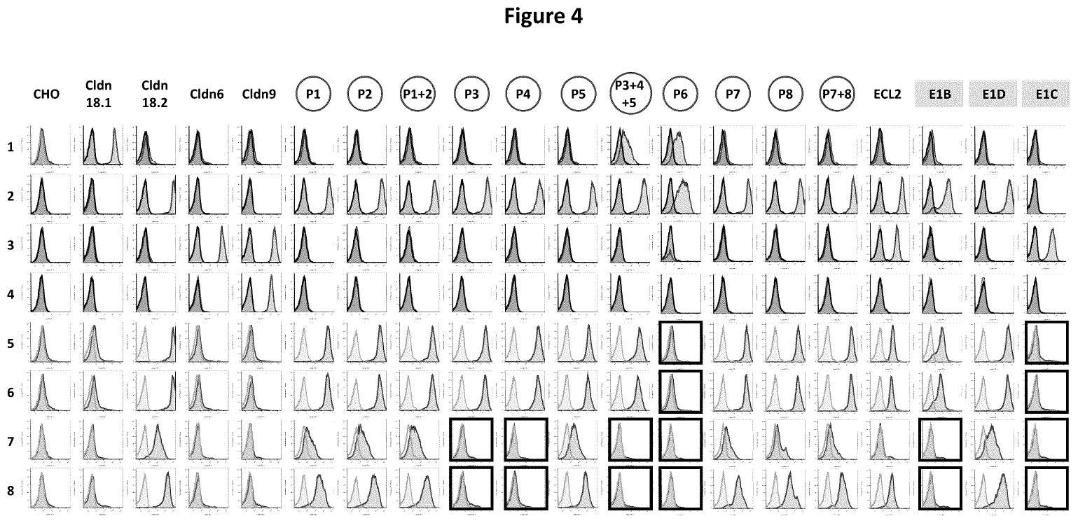

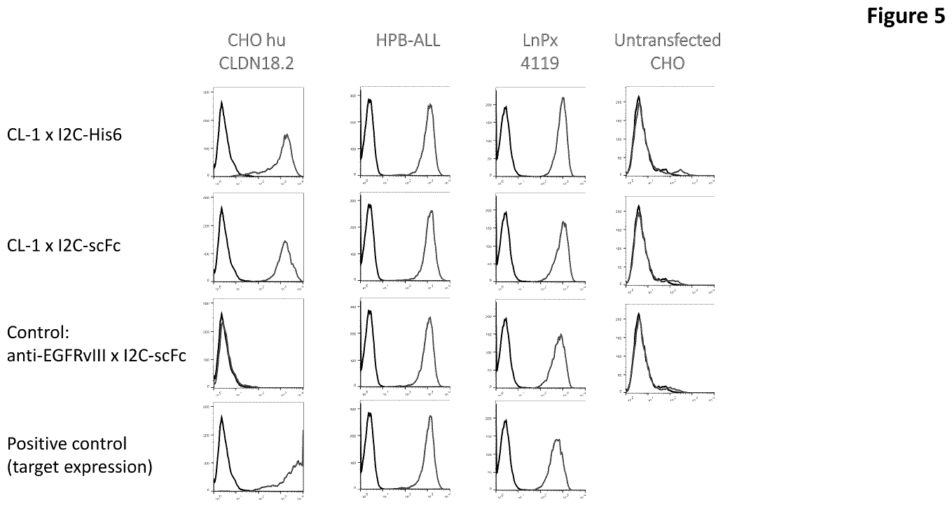

According to one embodiment, the first domain of the antibody construct of the invention binds to human CLDN18.2 on the surface of a target cell, wherein Glu (E) at position 56 of human CLDN18.2 is essential for the binding of the first domain, and Ala (A) at position 42 and/or Asn (N) at position 45 of human CLDN18.2 is/are not essential for the binding of the first domain. In this context, the term “is essential for the binding” means that the specified amino acid (Glu at position 56) is necessary for the binding of the antibody construct to CLDN18.2 to occur. If the amino acid Glu at position 56 is exchanged, e.g. for Gln (→E56Q), binding of the antibody construct is abolished or significantly decreased (see FIG. 4, column denominated “P6”). The term “is not essential for the binding” means that the specified amino acid (Ala at position 42 and/or Asn at position 45) is not necessary for the binding of the antibody construct to CLDN18.2 to occur. If the amino acid Ala at position 42 and/or Asn at position 45 is exchanged, e.g. for Ser at position 42 (→A42S) and/or Gln at position 45 (→N45Q), binding of the antibody construct is not or not significantly decreased (see FIG. 4, columns denominated “P3” and “P4”).

According to one embodiment, the first domain of the antibody construct of the invention comprises a VH region comprising CDR-H1, CDR-H2 and CDR-H3 and a VL region comprising CDR-L1, CDR-L2 and CDR-L3 selected from the group consisting of:
a) CDR-H1 as depicted in SEQ ID NO: 121, CDR-H2 as depicted in SEQ ID NO: 122, and CDR-H3 as depicted in SEQ ID NO: 123, CDR-L1 as depicted in SEQ ID NO: 124, CDR-L2 as depicted in SEQ ID NO: 125 and CDR-L3 as depicted in SEQ ID NO: 126; and
b) CDR-H1 as depicted in SEQ ID NO: 133, CDR-H2 as depicted in SEQ ID NO: 134, and CDR-H3 as depicted in SEQ ID NO: 135, and CDR-L1 as depicted in SEQ ID NO: 136, CDR-L2 as depicted in SEQ ID NO: 137 and CDR-L3 as depicted in SEQ ID NO: 138.

In the context of the present invention, the term “variable” refers to those portions of antibody or immunoglobulin domains that exhibit variability in their sequence and that are involved in determining the specificity and binding affinity of a particular antibody (i.e., the “variable region(s)”). Usually, the pairing of a heavy chain variable region (VH) and a light chain variable region (VL) together forms a single antigen-binding site.

Variability is not evenly distributed throughout the variable regions of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable regions are called the “framework” (FR) regions and provide a scaffold for the six CDRs in three-dimensional space to form an antigen-binding surface. The variable regions of naturally occurring antibody heavy and light chains each comprise four FR regions (FR1, FR2, FR3, and FR4), largely adopting a β-sheet configuration. Together with the CDRs, they form the following sequence within a variable heavy or light chain: FR1-CDR1-FR2-CDR2-FR3-CDR3-FR4. The hypervariable regions in each chain are held together in close proximity by the framework regions and, usually together with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody (or antibody construct or binding domain) with the antigen and hence contribute to the functional activity of an antibody molecule: they are the main determinants of antigen specificity.

The exact definition of CDR boundaries and lengths is subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Corresponding loops between antibodies may, therefore, have very similar three-dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable region in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate class sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibodies or antibody constructs/binding domains, the heavy chain CDR3 appears to constitute the major area of contact between the antigen and the antibody. In vitro selection schemes in which CDR3 alone is varied can be used to vary the binding properties of an antibody or antibody construct/binding domain or determine which residues contribute to the binding of an antigen. Hence, CDR3 is typically the greatest source of molecular diversity within the antibody binding site. CDR-H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The heavy chain constant (CH) domain most proximal to VH is usually designated as CH1. The constant (“C”) domains are not directly involved in antigen binding, but exhibit various effector functions, such as antibody-dependent cell-mediated cytotoxicity (ADCC) and complement activation (complement dependent cytotoxicity, CDC). The Fc region of an antibody is the “tail” region of a classical antibody that interacts with cell surface receptors called Fc receptors and some proteins of the complement system. In IgG, IgA and IgD antibody isotypes, the Fc region is composed of two identical protein fragments, derived from the second and third constant domains (CH2 and CH3) of the antibody's two heavy chains. IgM and IgE Fc regions contain three heavy chain constant domains (CH2, CH3 and CH4) in each polypeptide chain. The Fc regions also contains part of the so-called “hinge” region held together by one or more disulfides and noncovalent interactions. The Fc region of a naturally occurring IgG bears a highly conserved N-glycosylation site. Glycosylation of the Fc fragment is essential for Fc receptor-mediated activity.

ADCC is a mechanism of cell-mediated immune defense whereby an effector cell of the immune system actively lyses a target cell, whose membrane-surface antigens have been bound by specific antibodies. ADCC requires an immune effector cell which classically is known to be a natural killer (NK) cell that typically interacts with IgG antibodies. However, ADCC can also be mediated by macrophages, neutrophils and eosinophils. ADCC involves activation of effector cells expressing Fc receptors by antibodies expressing an Fc portion. For example, the most common Fc receptor on the surface of an NK cell is calles CD16 or FcγRIII. Once the Fc receptor binds to the Fc region of IgG, the NK cell releases cytotoxic factors that cause the death of the target cell. Likewise, the Fc receptor (FceRI) of an eosinophil will recognize IgE. In CDC, in contrast, the molecule “Clq” of the complement system binds to the antibody Fc region, and this binding triggers the complement cascade which leads to the formation of the membrane attack complex (MAC) at the surface of the target cell, as a result of the classical pathway complement activation. In therapeutic antibodies or antibody constructs, both ADCC and CDC can be modulated by Fc isotype engineering, Fc genetic mutations, or Fc glycosylation profile modifications.

The sequence of antibody genes after assembly and somatic mutation is highly varied, and these varied genes are estimated to encode 1010 different antibody molecules (Immunoglobulin Genes, 2^{nd} ed., eds. Jonio et al., Academic Press, San Diego, C A, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

It is envisaged that the antibody construct has a cysteine clamp within the first domain. This cysteine clamp may be introduced to improve stability of the construct. See e.g. US 2016/0193295.

In one embodiment of the invention, the first domain of the antibody construct of the invention comprises a VH region having an amino acid sequence as depicted in SEQ ID NO: 127 or SEQ ID NO: 139. In a further embodiment, the first domain of the antibody construct of the invention comprises a VL region having an amino acid sequence as depicted in SEQ ID NO: 128 or SEQ ID NO: 140. In another embodiment, the first domain of the antibody construct of the invention comprises a VH region and a VL region having an amino acid sequence as depicted in SEQ ID NOs: 127+128 (VH+VL) or SEQ ID NOs: 139+140 (VH+VL). In yet a further embodiment, the first domain of the antibody construct of the invention comprises a polypeptide having an amino acid sequence as depicted in SEQ ID NO: 129 or SEQ ID NO: 141.

As described herein above, the invention provides an embodiment wherein the antibody construct is in a format selected from the group consisting of (scFv)₂, scFv-single domain mAb, diabodies and oligomers of any of the afermentioned formats. The term “is in a format” does not exclude that the construct can be further modified, e.g. by attachment or fusion to other moieties, as described herein. According to one embodiment of the antibody construct of the present invention, the first and/or the second domain are in the format of an scFv. In an scFv, the VH region and the and VL region are arranged in the order VH-VL or VL-VH (from N- to C-terminus). It is envisaged that the VH and the VL regions of the first and/or the second binding domain are connected via a linker, preferably a peptide linker. According to one embodiment of the first and/or the second domain, the VH-region is positioned N-terminally of the linker, and the VL-region is positioned C-terminally of the linker. In other words, in one embodiment of the first and/or the second domain, the scFv comprises from the N-terminus to the C-terminus: VH-linker-VL. It is furthermore envisaged that the first domain and the second domain of the antibody construct are connected via a linker, preferably a peptide linker. The antibody construct may e.g. comprise the domains in the order (from N-terminus to C-terminus) first domain—linker—second domain. The inverse order (second domain—linker—first domain) is also possible.

The linkers are preferably peptide linkers, more preferably short peptide linkers. In accordance with the present invention, a “peptide linker” comprises an amino acid sequence which connects the amino acid sequences of one domain with another (variable and/or binding) domain (e.g. a variable domain or a binding domain) of the antibody construct. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct of the invention. Examples of useful peptide linkers are shown in SEQ ID NOs: 155-168. In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13, and the linker between the two binding domains may have a length between 3 and 10 amino acids, preferably between 4 and 8. It is further envisaged that the peptide linkers are glycine/serine linkers, such as those depicted in SEQ ID NOs: 156 and 158-168. The majority of the amino acids in glycine/serine linkers are selected from glycine and serine.

In the event that a linker is used, this linker is preferably of a length and sequence sufficient to ensure that each of the first and second domains can, independently from one another, retain their differential binding specificities. For peptide linkers which connect the at least two binding domains (or the two variable regions forming one binding domain) in the antibody construct, those peptide linkers are envisaged which comprise only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A “single amino acid” linker in the context of said “peptide linker” is Gly. Another embodiment of a peptide linker is characterized by the amino acid sequence Gly-Gly-Gly-Gly-Ser, i.e. Gly Ser (SEQ ID NO: 156), or polymers thereof, i.e. (Gly₄Ser)x, where x is an integer of 1 or greater (e.g. 2 or 3). Usable linkers are depicted in SEQ ID NOs: 155-163. The characteristics of said peptide linkers are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which do not promote any secondary structures are preferred. The linkage of said domains to each other can be provided, e.g., by genetic engineering. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

According to one embodiment of the invention, the antibody construct of the invention is a “single chain antibody construct”. It is also envisaged that either the first or the second or both binding domains may be in the format of a “single chain Fv” (scFv). Although the two domains of the Fv fragment, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for producing single chain antibody constructs (see, inter alia, U.S. Pat. No. 4,946,778, Kontermann and Dübel (2010), loc. cit. and Little (2009), loc. cit.) can be adapted to produce single chain antibody constructs specifically recognizing (an) elected target(s).

Bivalent (also called divalent) or bispecific single-chain variable fragments (bi-scFvs or di-scFvs) having the format (scFv)₂ can be engineered by linking two scFv molecules (e.g. with linkers as described hereinbefore). The linking can be done by producing a single polypeptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. In this case, the VH and the VL of a binding domain (binding either to the target antigen CLDN18.2 or to CD3) are not directly connected via a peptide linker. Thus, the VH of the CD3 binding domain may e.g. be fused to the VL of the CLDN18.2 binding domain via a peptide linker, and the VH of the CLDN18.2 binding domain is fused to the VL of the CD3 binding domain via such peptide linker. This type is known as diabodies (see e.g. Hollinger, Philipp et al., (July 1993) Proceedings of the National Academy of Sciences of the United States of America 90 (14): 6444-8).

Antibody constructs denominated “single domain antibodies” comprise one (monomeric) antibody variable region which is able to bind selectively to a specific antigen, independently of other variable regions. The first single domain antibodies were engineered from heavy chain antibodies found in camelids, and these are called VHH fragments. Cartilaginous fishes also have heavy chain antibodies (IgNAR) from which single domain antibodies called V_{NAR} fragments can be obtained. An alternative approach is to split the dimeric variable regions from common immunoglobulins into monomers, hence obtaining VH or VL as a single domain Ab. Although most research into single domain antibodies is currently based on heavy chain variable regions, nanobodies derived from light chains were also shown to bind specifically to target epitopes. Examples of single domain antibodies are called sdAb, nanobodies or single variable domain antibodies. A (single domain mAb)₂ is hence a monoclonal antibody construct composed of (at least) two single domain monoclonal antibody constructs, which are individually selected from the group comprising VH, VL, V_{H}H and V_{NAR}. The linker is preferably in the form of a peptide linker. Similarly, an “scFv-single domain mAb” is a monoclonal antibody construct composed of at least one single domain antibody as described above and one scFv molecule as described above. Again, the linker is preferably in the form of a peptide linker.

It is also envisaged that the antibody construct of the invention has, in addition to its function to bind to the target molecules CLDN18.2 and CD3, a further function. In this format, the antibody construct may be a trifunctional or multifunctional antibody construct by targeting target cells through CLDN18.2 binding, mediating cytotoxic T cell activity through CD3 binding and providing a further function such as means or domains to enhance or extend serum half-life, a fully functional or modified Fc constant domain mediating ADCC through recruitment of effector cells, a label (fluorescent etc.), a therapeutic agent such as a toxin or radionuclide, etc.

Examples for means or domains to extend serum half-life of the antibody constructs of the invention include peptides, proteins or domains of proteins, which are fused or otherwise attached to the antibody constructs. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the antibody constructs of the present invention. The concept of attaching larger domains of proteins or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may e.g. be optimized/modified in order to allow the desired pairing of dimers or mulimers, to abolish Fc receptor binding (e.g. to avoid ADCC or CDC) or for other reasons. A further concept known in the art to extend the half-life of substances or molecules in the human body is the pegylation of those molecules (such as the antibody constructs of the present invention).

In one embodiment, the antibody constructs according to the invention are linked (e.g. via peptide bond) with a fusion partner (such as a protein, polypeptide or peptide), e.g. for the purpose of extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. Exempary sequences of these fusion partners are depticed in SEQ ID NOs: 170-232. In general, the fusion partners may be linked to the N-terminus or to the C-terminus of the antibody constructs according to the invention, either directly (e.g. via peptide bond) or through a peptide linker such as (GGGGS), (wherein “n” is an integer of 2 or greater, e.g. 2 or 3 or 4). Suitable peptide linkers are depticed in SEQ ID NOs: 155-163.

Hence, it is envisaged that an antibody construct according to the present invention comprises:
(a) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
   a peptide linker having an amino acid sequence selected from the group consisting of SEQ ID NOs: 155-163; and
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168; and
   optionally a His-tag, e.g. selected from SEQ ID NOs: 169, 269, 270 or 271;
(b) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
   a peptide linker having an amino acid sequence selected from the group consisting of SEQ ID NOs: 155-163;
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168;
   optionally a peptide linker having an amino acid sequence selected from the group consisting of SEQ ID NOs: 155-163;
   a polypeptide having an amino acid sequence selected from the group consisting of SEQ ID NOs: 170 and 176-205; and
   optionally a His-tag, e.g. selected from SEQ ID NOs: 169, 269, 270 or 271;
(c) a polypeptide comprising in the following order from N-terminus to C-terminus:
   a polypeptide having the amino acid sequence QRFVTGHFGGLX₁PANG (SEQ ID NO: 171) wherein X₁ is Y or H; and
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
   a peptide linker having an amino acid sequence selected from the group consisting of SEQ ID NOs: 155-163;
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168;
   a polypeptide having an amino acid sequence as depicted in SEQ ID NO: 173 or SEQ ID NO: 175; and
   optionally a His-tag, e.g. selected from SEQ ID NOs: 169, 269, 270 or 271;
(d) a polypeptide comprising in the following order from N-terminus to C-terminus:
   a polypeptide having an amino acid sequence selected from the group consisting of SEQ ID NO: 34, SEQ ID NO: 43, SEQ ID NO: 52, SEQ ID NO: 61, SEQ ID NO: 70, SEQ ID NO: 79, SEQ ID NO: 88, SEQ ID NO: 97, SEQ ID NO: 106, SEQ ID NO: 115, and SEQ ID NO: 118;
   a peptide linker having the amino acid sequence depicted in SEQ ID NO: 162;
   a polypeptide having an amino acid sequence selected from the group consisting of SEQ ID NO: 128 and SEQ ID NO: 140, followed by a serine residue at the C-terminus;
   a polypeptide having the amino acid sequence depicted in SEQ ID NO: 206; and a polypeptide comprising in the following order from N-terminus to C-terminus:
   a polypeptide having an amino acid sequence selected from the group consisting of SEQ ID NO: 127 and SEQ ID NO: 139;
   a peptide linker having the amino acid sequence depicted in SEQ ID NO: 162;
   a polypeptide having an amino acid sequence selected from the group consisting of SEQ ID NO: 35, SEQ ID NO: 44, SEQ ID NO: 53, SEQ ID NO: 62, SEQ ID NO: 71, SEQ ID NO: 80, SEQ ID NO: 89, SEQ ID NO: 98, SEQ ID NO: 107, SEQ ID NO: 116, and SEQ ID NO: 119; followed by a serine residue at the C-terminus; and
   a polypeptide having the amino acid sequence depicted in SEQ ID NO: 207;
(e) a polypeptide comprising in the following order from N-terminus to C-terminus:
   a polypeptide having an amino acid sequence selected from the group consisting of SEQ ID NO: 34, SEQ ID NO: 43, SEQ ID NO: 52, SEQ ID NO: 61, SEQ ID NO: 70, SEQ ID NO: 79, SEQ ID NO: 88, SEQ ID NO: 97, SEQ ID NO: 106, SEQ ID NO: 115, and SEQ ID NO: 118;
   a peptide linker having the amino acid sequence depicted in SEQ ID NO: 162;
   a polypeptide having an amino acid sequence selected from the group consisting of SEQ ID NO: 128 and SEQ ID NO: 140; and
   a polypeptide having the amino acid sequence depicted in SEQ ID NO: 208; and
   a polypeptide comprising in the following order from N-terminus to C-terminus:
   a polypeptide having an amino acid sequence selected from the group consisting of SEQ ID NO: 127 and SEQ ID NO: 139;
   a peptide linker having the amino acid sequence depicted in SEQ ID NO: 162;
   a polypeptide having an amino acid sequence selected from the group consisting of SEQ ID NO: 35, SEQ ID NO: 44, SEQ ID NO: 53, SEQ ID NO: 62, SEQ ID NO: 71, SEQ ID NO: 80, SEQ ID NO: 89, SEQ ID NO: 98, SEQ ID NO: 107, SEQ ID NO: 116, and SEQ ID NO: 119 followed by a serine residue at the C-terminus; and
   a polypeptide having the amino acid sequence depicted in SEQ ID NO: 209;
(f) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
   a peptide linker having an amino acid sequence selected from the group consisting of SEQ ID NOs: 155-163;
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168;
   a polypeptide having the amino acid sequence depicted in SEQ ID NO: 210; and
   a polypeptide having the amino acid sequence depicted in SEQ ID NO: 211;
(g) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168; and
   a polypeptide having the amino acid sequence depicted in SEQ ID NO: 212; and
   a polypeptide comprising in the following order starting from the N-terminus:
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168; and
   a polypeptide having the amino acid sequence depicted in SEQ ID NO: 213;
(h) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168; and
   a polypeptide having the amino acid sequence depicted in SEQ ID NO: 214; and a polypeptide comprising in the following order starting from the N-terminus:
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168; and
   a polypeptide having the amino acid sequence depicted in SEQ ID NO: 215;
or
(i) a polypeptide comprising in the following order from N-terminus to C-terminus:
   a polypeptide having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
   a peptide linker having an amino acid sequence selected from the group consisting of SEQ ID NOs: 155-163;
   a polypeptide having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168; and
   a polypeptide having the amino acid sequence depicted in SEQ ID NO: 216.

According to another embodiment, the antibody construct of the invention comprises (in addition to the first and second domain) a third domain which comprises two polypeptide monomers, each comprising a hinge, a CH2 and a CH3 domain, wherein said two polypeptide monomers are fused to each other via a peptide linker. It is envisaged that said third domain comprises in an N-terminal to C-terminal order: hinge-CH2-CH3-linker-hinge-CH2-CH3. Amino acid sequences that can be used for said third domain are depicted in SEQ ID NOs: 225-232. Each of said polypeptide monomers can have an amino acid sequence that is selected from the group consisting of SEQ ID NOs: 217-224, or that is at least 90% identical to those sequences. In another embodiment, the first and second domains of the antibody construct of the invention are fused to the third domain via a peptide linker which is for example selected from the group consisting of SEQ ID NO: 155, 156, 157, 158, 159, 160, 161, 162 or 163.

In line with the present invention, a “hinge” is an IgG hinge region. This region can be identified by analogy using the Kabat numbering, see e.g. Kabat positions 223-243. In line with the above, the minimal requirement for a “hinge” are the amino acid residues corresponding to the IgG₁ sequence stretch of D231 to P243 according to the Kabat numbering. The terms “CH2” and “CH3” refer to the immunoglobulin heavy chain constant regions 2 and 3. These regions can as well be identified by analogy using the Kabat numbering, see e.g. Kabat positions 244-360 for CH2 and Kabat positions 361-478 for CH3. Is is understood that there is some variation between the immunoglobulins in terms of their IgG₁ Fc region, IgG₂ Fc region, IgG₃ Fc region, IgG₄ Fc region, IgM Fc region, IgA Fc region, IgD Fc region and IgE Fc region (see, e.g., Padlan, Molecular Immunology, 31(3), 169-217 (1993)). The term Fc region refers to the last two heavy chain constant regions of IgA, IgD, and IgG, and the last three heavy chain constant regions of IgE and IgM. The Fc region can also include the flexible hinge N-terminal to these domains. For IgA and IgM, the Fc region may include the J chain. For IgG, the Fc region comprises immunoglobulin domains CH2 and CH3 and the hinge between the first two domains and CH2. Although the boundaries of the Fc region of an immunoglobulin may vary, an example for a human IgG heavy chain Fc portion comprising a functional hinge, CH2 and CH3 domain can be defined e.g. to comprise residues D231 (of the hinge domain) to P476 (of the C-terminus of the CH3 domain), or D231 to L476, respectively, for IgG₄, wherein the numbering is according to Kabat.

The antibody construct of the invention may hence comprise in an N- to C-terminal order:
(a) the first domain;
(b) a peptide linker having an amino acid sequence selected from the group consisting of SEQ ID NOs: 156, 162 and 163;
(c) the second domain;
(d) a peptide linker having an amino acid sequence selected from the group consisting of SEQ ID NOs: 155, 156, 158, 159, 160, 162, and 163;
(e) the first polypeptide monomer of the third domain (comprising a hinge, a CH2 and a CH3 domain);
(f) a peptide linker having an amino acid sequence selected from the group consisting of SEQ ID NOs: 165, 166, 167, and 168; and
(g) the second polypeptide monomer of the third domain (comprising a hinge, a CH2 and a CH3 domain).

It is also envisaged that the antibody construct of the invention comprises in an N- to C-terminal order:
the first domain having an amino acid sequence selected from the group consisting of SEQ ID NO: 129 and SEQ ID NO: 141; wherein the peptide linker comprised within those sequences and having SEQ ID NO: 163 can be replaced by any one of SEQ ID NOs: 155-162 and 164-168;
a peptide linker having an amino acid sequence selected from the group consisting of SEQ ID NOs: 156, 162, and 163;
the second domain having an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255; wherein the peptide linker comprised within those sequences and having SEQ ID NO: 163 can be replaced by any one of SEQ ID NOs: 155-162 and 164-168;
a peptide linker having an amino acid sequence selected from the group consisting of SEQ ID NOs: 155, 156, 158, 159, 160, 162, and 163; and
the third domain having an amino acid sequence selected from the group consisting of SEQ ID NOs: 225-232.

Hence, in one embodiment, the antibody construct of the present invention comprises or consists of a polypeptide having an amino acid sequence selected from the group of those depicted in SEQ ID NO: 131, SEQ ID NO: 132, SEQ ID NO: 143 or in SEQ ID NO: 144.

Covalent modifications of the antibody constructs are also included within the scope of this invention, and are generally, but not always, done post-translationally. For example, several types of covalent modifications of the antibody construct are introduced into the molecule by reacting specific amino acid residues of the antibody construct with an organic derivatizing agent that is capable of reacting with selected side chains or with the N- or C-terminal residues. Derivatization with bifunctional agents is useful for crosslinking the antibody constructs of the present invention to a water-insoluble support matrix or surface for use in a variety of methods. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antibody constructs included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an antibody construct may be altered through changes at the DNA level, particularly by mutating the DNA encoding the polypeptide at preselected bases such that codons are generated that will translate into the desired amino acids.

Another means of increasing the number of carbohydrate moieties on the antibody construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antibody construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the antibody construct are also contemplated herein. For example, another type of covalent modification of the antibody construct comprises linking the antibody construct to various non-proteinaceous polymers, including polyols, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antibody construct, e.g. in order to facilitate the addition of polymers such as polyethylene glycol (PEG).

In some embodiments, the covalent modification of the antibody constructs of the invention comprises the addition of one or more labels. The labelling group may be coupled to the antibody construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzymatic groups (e.g. horseradish peroxidase, β-galactosidase, luciferase, alkaline phosphatase) f) biotinylated groups
g) predetermined polypeptide epitopes recognized by a secondary reporter (e.g., leucine zipper pair sequences, binding sites for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank® Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994. FEBS Letters 344:191. The use of a modified leucine zipper that allows for stable trimerization of a heterologous protein fused thereto is described in Fanslow et al., 1994, Semin. Immunol. 6:267-78.

The antibody construct of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag. GST-tag, chitin binding domain (CBD-tag), maltose binding protein (MBP-tag), Flag-tag, Strep-tag and variants thereof (e.g. StrepII-tag) and His-tag. All herein disclosed antibody constructs characterized by the identified CDRs may comprise a His-tag domain, which is generally known as a repeat of consecutive His residues in the amino acid sequence of a molecule, e.g. of five His residues (SEQ ID NO: 269), or of six His residues (hexa-histidine, SEQ ID NO: 169). The His-tag may be located e.g. at the N- or C-terminus of the antibody construct. In one embodiment, a hexa-histidine tag (HHHHHH) is linked via peptide bond to the C-terminus of the antibody construct according to the invention.

It is also envisaged that the antibody construct of the present invention comprises or consists of a polypeptide which has an amino acid sequence selected from the group consisting of those depicted in SEQ ID NOs: 130 and 142, and which is linked at its N-terminus or at its C-terminus with a protein purification tag, preferably via a peptide bond (amide bond). The linking of the protein purification tag at the C-terminus of the polypeptide is preferred. It is envisaged that the protein purification tag is a short peptide. For example, the length of the short peptide may be 2-30 amino acids, 4-25 amino acids, 5-20 amino acids or 6-19 amino acids. Examples of protein purification tags include, but are not limited to, AU1 epitope (e.g. as depicted in SEQ ID NO: 272), AU5 epitope (e.g. as depicted in SEQ ID NO: 273), T7-tag (e.g. as depicted in SEQ ID NO: 274), V5-tag (e.g. as depicted in SEQ ID NO: 275), B-tag (e.g. as depicted in SEQ ID NO: 276), E2 epitope (e.g. as depicted in SEQ ID NO: 277), FLAG epitope/FLAG tag (e.g. as depicted in SEQ ID NO: 278), Glu-Glu tag (e.g. as depicted in SEQ ID NOs: 279 or 280), HA tag, Histidine affinity tag (e.g. as depicted in SEQ ID NO: 281), HSV epitope (e.g. as depicted in SEQ ID NO: 282), KT3 epitope (e.g. as depicted in SEQ ID NO: 283), Myc epitope (e.g. as depicted in SEQ ID NO: 284), polyarginine tag (5-6 Arg residues), polyaspartate tag (5-16 Asp residues), polyhistidine tag (2-10 His residues, usually 6 His residues, see e.g. SEQ ID NOs: 169 and 269-271), polyphenylalanine tag (usually 11 Phe residues), S1 tag (e.g. as depicted in SEQ ID NO: 285), S-tag (e.g. as depicted in SEQ ID NO: 286), Strep-tag (e.g. as depicted in SEQ ID NOs: 287 or 288), universal tag (e.g. as depicted in SEQ ID NO: 289), VSV-G (e.g. as depicted in SEQ ID NO: 290), Protein C (e.g. as depicted in SEQ ID NO: 291), and Protein A. A histidine tag is preferred, especially a 6×His tag (SEQ ID NO: 169). Is it hence further envisaged that the antibody construct of the present invention consists of a polypeptide which has an amino acid sequence selected from the group consisting of those depicted in SEQ ID NOs: 130 and 142, and which is linked at its C-terminus with a 6×His tag via a peptide bond. An embodiment of the antibody construct of the present invention has an amino acid sequence as depicted in SEQ ID NO: 131 or SEQ ID NO: 143.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (a) and beta (B) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The antibody construct of the invention comprises a domain which binds to CD3 on the surface of a T cell. “CD3” (cluster of differentiation 3) is a T cell co-receptor composed of four chains. In mammals, the CD3 protein complex contains a CD3γ (gamma) chain, a CD38 (delta) chain, and two CD3ε (epsilon) chains. These four chains associate with the T cell receptor (TCR) and the so-called ζ (zeta) chain to for the “T cell receptor complex” and to generate an activation signal in T lymphocytes. The CD3γ (gamma), CD38 (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide which in humans is encoded by the CD3E gene which resides on chromosome 11.

The redirected lysis of target cells via the recruitment of T cells by an antibody construct which binds to CD3 on the T cell and to a target protein on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see e.g. WO 2007/042261.

Cytotoxicity mediated by CLDN18.2×CD3 antibody constructs can be measured in various ways. See Example 7. The “half maximal effective concentration” (EC₅₀) is commonly used as a measure of potency of a biologically active molecule such as an antibody construct of the present invention. It may be expressed in molar units. In the present case of measuring cytotoxicity, the EC₅₀ value refers to the concentration of an antibody construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). An EC₅₀ value may typically be expected to be lower when stimulated/enriched CD8+ T cells are used as effector cells, compared with unstimulated PBMC. If the target cells are of macaque origin or express or are transfected with macaque CLDN18.2, the effector cells should also be of macaque origin, such as a macaque T cell line, e.g. 4119LnPx. The target cells should express CLDN18.2, such as human or macaque CLDN18.2, on the cell surface. Preferably the target cells should express at least the extracellular loop(s) of CLDN18.2, such as CLDN18.2 loop 1 and/or loop 2, on the cell surface. Target cells can be a cell line (such as CHO) which is stably or transiently transfected with CLDN18.2, e.g. human or macaque CLDN18.2. Alternatively, the target cells can be a CLDN18.2 positive natural expresser cell line, such as the human gastric cancer lines SNU-601 or SNU-620, or also SNU-16, NUGC, NUG-C4, GSU or IM95. Usually EC₅₀ values are expected to be lower when using target cells that express higher levels of CLDN18.2 on the cell surface compared with target cells having a lower target expression rate.

The effector to target cell (E:T) ratio in a cytotoxicity assay is usually about 10:1, but can also vary. Cytotoxic activity of CLDN18.2×CD3 antibody constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by CLDN18.2×CD3 antibody constructs of the present invention is measured in a cell-based cytotoxicity assay. It may also be measured in a 51-chromium release assay. It is envisaged that the EC₅₀ value of the antibody constructs of the invention is ≤300 pM, ≤280 pM, ≤260 pM, ≤250 pM, ≤240 pM, ≤220 pM, ≤200 pM, ≤180 pM, ≤160 pM, ≤150 PM, ≤140 pM, ≤120 pM, ≤100 pM, ≤90 pM, ≤80 pM, ≤70 pM, ≤60 pM, ≤50 pM, ≤40 pM, ≤30 PM, ≤20 PM, ≤15 pM, ≤10 pM, or ≤5 pM.

The above given EC₅₀ values can be measured in different assays and under different conditions. For example, when human PBMCs are used as effector cells and CLDN18.2 transfected cells such as CHO cells are used as target cells, it is envisaged that the EC₅₀ value of the CLDN18.2×CD3 antibody construct is ≤500 pM, ≤400 pM, ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 PM, ≤30 pM, ≤20 pM, ≤15 pM, ≤10 pM, or ≤5 pM. When human PBMCs are used as effector cells and when the target cells are a CLDN18.2 positive cell line such as SNU-601 or SNU-620, it is envisaged that the EC₅₀ value of the CLDN18.2×CD3 antibody construct is ≤300 pM, ≤280 pM, ≤260 pM, ≤250 PM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 PM, or ≤5 pM.

According to one embodiment, the CLDN18.2×CD3 antibody constructs of the present invention do not induce/mediate lysis or do not essentially induce/mediate lysis of cells that do not express CLDN18.2 on their surface (CLDN18.2 negative cells), such as CHO cells. The term “do not induce lysis”, “do not essentially induce lysis”, “do not mediate lysis” or “do not essentially mediate lysis” means that an antibody construct of the present invention does not induce or mediate lysis of more than 30%, preferably not more than 20%, more preferably not more than 10%, particularly preferably not more than 9%, 8%, 7%, 6% or 5% of CLDN18.2 negative cells, whereby lysis of CLDN18.2 expressing target cells (such as cells transformed or transfected with CLDN18.2 or a natural expresser cell line such as the human gastric cancer lines SNU-601 or SNU-620) is set to be 100%. This usually applies for concentrations of the antibody construct of up to 500 nM. Cell lysis measurement is a routine technique. Moreover, the present specification teaches specific instructions how to measure cell lysis.

The difference in cytotoxic activity between the monomeric and the dimeric isoform of individual CLDN18.2×CD3 antibody constructs is referred to as “potency gap”. This potency gap can e.g. be calculated as ratio between EC₅₀ values of the molecule's monomeric and dimeric form. In one method to determine this gap, an 18 hour 51-chromium release assay or a 48 h FACS-based cytotoxicity assay is carried out as described hereinbelow (Examples 7.1 and 7.2) with purified antibody construct monomer and dimer. Effector cells are stimulated enriched human CD8+ T cells or unstimulated human PBMC. Target cells are hu CLDN18.2 transfected CHO cells. Effector to target cell (E:T) ratio is 10:1. Potency gaps of the CLDN18.2×CD3 antibody constructs of the present invention are preferably ≤5, more preferably ≤4, even more preferably ≤3, even more preferably ≤2 and most preferably ≤1.

The first and/or the second domain of the antibody construct of the invention is/are preferably cross-species specific for members of the mammalian order of primates, such as macaques. Cross-species specific CD3 binding domains are, for example, described in WO 2008/119567. According to one embodiment, the second domain, in addition to binding to human CD3, will also bind to CD3 of primates including (but not limited to) new world primates (such as Callithrix jacchus, Saguinus Oedipus or Saimiri sciureus), old world primates (such as baboons and macaques), gibbons, orangutans and non-human homininde. It is envisaged that the second domain which binds to human CD3 on the surface of a T cell also binds at least to macaque CD3. A preferred macaque is Macaca fascicularis. Macaca mulatta (Rhesus) is also envisaged. One antibody construct of the invention comprises a first domain which binds to human CLDN18.2 on the surface of a target cell and a second domain which binds to human CD3 on the surface of a T cell and at least macaque CD3.

In one embodiment, the affinity gap of the antibody constructs according to the invention for binding macaque CD3 versus human CD3 [KD ma CD3:KD hu CD3] (as determined e.g. by BiaCore or by Scatchard analysis) is between 0.01 and 100, preferably between 0.1 and 10, more preferably between 0.2 and 5, more preferably between 0.3 and 4, even more preferably between 0.5 and 3 or between 0.5 and 2.5, and most preferably between 0.5 and 1. See Example 3.

The second domain of the antibody construct of the invention binds to CD3. More preferably, it binds to CD3 on the surface of a T cell. It is furthermore envisaged that the second domain binds to human CD3, preferably to human CD3 on the surface of a T cell. It is also envisaged that the second domain binds to CD3 epsilon. More preferably, it binds to human CD3 epsilon, e.g. to human CD3 epsilon on the surface of a T cell. A preferred amino acid sequence for the extracellular domain of human CD3 epsilon is depicted in SEQ ID NO: 256.

In one embodiment of the present invention, the second domain of the antibody construct binds to human CD3 epsilon (or human CD3 epsilon on the surface of a T cell) and to Callithrix jacchus or Saimiri sciureus CD3 epsilon. It is also envisaged that the second domain binds to an extracellular epitope of CD3 epsilon, preferably to an extracellular epitope of human CD3 epsilon. It is also envisaged that the second domain binds to an extracellular epitope of the human and the Macaca CD3 epsilon chain. One preferred epitope of CD3 epsilon is comprised within amino acid residues 1-27 of the human CD3 epsilon extracellular domain (see SEQ ID NO: 257). Even more specifically, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or bispecific antibody constructs directed against (human) CD3 or specifically against CD3 epsilon are known in the art, and their CDRs, VH and VL sequences can serve as a basis for the second binding domain of the antibody construct of the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific antibody constructs directed against a (cancer) target and CD3 are also being developed and (pre-)clinically tested, and their CD3 binding domain (CDRs, VH, VL) may serve as a basis for the second binding domain of the antibody construct of the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, RO6958688), AFM11, and MGD006 (S80880). Other examples of CD3 binding domains are disclosed e.g. in U.S. Pat. No. 7,994,289 B2, U.S. Pat. No. 7,728,114 B2, U.S. Pat. No. 7,381,803 B1, U.S. Pat. No. 6,706,265 B1.

It is envisaged for the antibody construct of the present invention that the second domain which binds to CD3 on the surface of a T cell comprises a VL region comprising CDR-L1, CDR-L2 and CDR-L3 selected from:
(a) CDR-L1 as depicted in SEQ ID NO: 37, CDR-L2 as depicted in SEQ ID NO: 38, and CDR-L3 as depicted in SEQ ID NO: 39;
(b) CDR-L1 as depicted in SEQ ID NO: 82, CDR-L2 as depicted in SEQ ID NO: 83, and CDR-L3 as depicted in SEQ ID NO: 84; and
(c) CDR-L1 as depicted in SEQ ID NO: 100, CDR-L2 as depicted in SEQ ID NO: 101, and CDR-L3 as depicted in SEQ ID NO: 102.

It is also envisaged for the antibody construct of the present invention that the second domain which binds to CD3 on the surface of a T cell comprises a VH region comprising CDR-H1, CDR-H2 and CDR-H3 selected from:
(a) CDR-H1 as depicted in SEQ ID NO: 31, CDR-H2 as depicted in SEQ ID NO: 32, and CDR-H3 as depicted in SEQ ID NO: 33;
(b) CDR-H1 as depicted in SEQ ID NO: 40, CDR-H2 as depicted in SEQ ID NO: 41, and CDR-H3 as depicted in SEQ ID NO: 42;
(c) CDR-H1 as depicted in SEQ ID NO: 49, CDR-H2 as depicted in SEQ ID NO: 50, and CDR-H3 as depicted in SEQ ID NO: 51;
(d) CDR-H1 as depicted in SEQ ID NO: 58, CDR-H2 as depicted in SEQ ID NO: 59, and CDR-H3 as depicted in SEQ ID NO: 60;
(e) CDR-H1 as depicted in SEQ ID NO: 67, CDR-H2 as depicted in SEQ ID NO: 68, and CDR-H3 as depicted in SEQ ID NO: 69;
(f) CDR-H1 as depicted in SEQ ID NO: 76, CDR-H2 as depicted in SEQ ID NO: 77, and CDR-H3 as depicted in SEQ ID NO: 78;
(g) CDR-H1 as depicted in SEQ ID NO: 85, CDR-H2 as depicted in SEQ ID NO: 86, and CDR-H3 as depicted in SEQ ID NO: 87;
(h) CDR-H1 as depicted in SEQ ID NO: 94, CDR-H2 as depicted in SEQ ID NO: 95, and CDR-H3 as depicted in SEQ ID NO: 96;
(i) CDR-H1 as depicted in SEQ ID NO: 103, CDR-H2 as depicted in SEQ ID NO: 104, and CDR-H3 as depicted in SEQ ID NO: 105; and
(j) CDR-H1 as depicted in SEQ ID NO: 112, CDR-H2 as depicted in SEQ ID NO: 113, and CDR-H3 as depicted in SEQ ID NO: 114.

It is furthermore envisaged for the antibody construct of the present invention that the second domain which binds to CD3 comprises a VL region comprising CDR-L1, CDR-L2 and CDR-L3 and a VH region comprising CDR-H1, CDR-H2 and CDR-H3 selected from:
(a) CDR-L1 as depicted in SEQ ID NO: 28, CDR-L2 as depicted in SEQ ID NO: 29, CDR-L3 as depicted in SEQ ID NO: 30, CDR-H1 as depicted in SEQ ID NO: 31, CDR-H2 as depicted in SEQ ID NO: 32, and CDR-H3 as depicted in SEQ ID NO: 33;
(b) CDR-L1 as depicted in SEQ ID NO: 37, CDR-L2 as depicted in SEQ ID NO: 38, CDR-L3 as depicted in SEQ ID NO: 39, CDR-H1 as depicted in SEQ ID NO: 40, CDR-H2 as depicted in SEQ ID NO: 41, and CDR-H3 as depicted in SEQ ID NO: 42;
(c) CDR-L1 as depicted in SEQ ID NO: 46, CDR-L2 as depicted in SEQ ID NO: 47, CDR-L3 as depicted in SEQ ID NO: 48, CDR-H1 as depicted in SEQ ID NO: 49, CDR-H2 as depicted in SEQ ID NO: 50, and CDR-H3 as depicted in SEQ ID NO: 51;
(d) CDR-L1 as depicted in SEQ ID NO: 55, CDR-L2 as depicted in SEQ ID NO: 56, CDR-L3 as depicted in SEQ ID NO: 57, CDR-H1 as depicted in SEQ ID NO: 58, CDR-H2 as depicted in SEQ ID NO: 59, and CDR-H3 as depicted in SEQ ID NO: 60;
(e) CDR-L1 as depicted in SEQ ID NO: 64, CDR-L2 as depicted in SEQ ID NO: 65, CDR-L3 as depicted in SEQ ID NO: 66, CDR-H1 as depicted in SEQ ID NO: 67, CDR-H2 as depicted in SEQ ID NO: 68, and CDR-H3 as depicted in SEQ ID NO: 69;
(f) CDR-L1 as depicted in SEQ ID NO: 73, CDR-L2 as depicted in SEQ ID NO: 74, CDR-L3 as depicted in SEQ ID NO: 75, CDR-H1 as depicted in SEQ ID NO: 76, CDR-H2 as depicted in SEQ ID NO: 77, and CDR-H3 as depicted in SEQ ID NO: 78;
(g) CDR-L1 as depicted in SEQ ID NO: 82, CDR-L2 as depicted in SEQ ID NO: 83, CDR-L3 as depicted in SEQ ID NO: 84, CDR-H1 as depicted in SEQ ID NO: 85, CDR-H2 as depicted in SEQ ID NO: 86, and CDR-H3 as depicted in SEQ ID NO: 87;
(h) CDR-L1 as depicted in SEQ ID NO: 91, CDR-L2 as depicted in SEQ ID NO: 92, CDR-L3 as depicted in SEQ ID NO: 93, CDR-H1 as depicted in SEQ ID NO: 94, CDR-H2 as depicted in SEQ ID NO: 95, and CDR-H3 as depicted in SEQ ID NO: 96;
(i) CDR-L1 as depicted in SEQ ID NO: 100, CDR-L2 as depicted in SEQ ID NO: 101, CDR-L3 as depicted in SEQ ID NO: 102, CDR-H1 as depicted in SEQ ID NO: 103, CDR-H2 as depicted in SEQ ID NO: 104, and CDR-H3 as depicted in SEQ ID NO: 105; and
(j) CDR-L1 as depicted in SEQ ID NO: 109, CDR-L2 as depicted in SEQ ID NO: 110, CDR-L3 as depicted in SEQ ID NO: 111, CDR-H1 as depicted in SEQ ID NO: 112, CDR-H2 as depicted in SEQ ID NO: 113, and CDR-H3 as depicted in SEQ ID NO: 114.

It is envisaged for the antibody construct of the present invention that the second domain which binds to CD3 on the surface of a T cell comprises a VL region selected from the group consisting of a VL region as depicted in any one of SEQ ID NO: 44, SEQ ID NO: 89, SEQ ID NO: 107, SEQ ID NO: 233, SEQ ID NO: 234 and SEQ ID NO: 235.

It is also envisaged that the second domain which binds to CD3 on the surface of a T cell comprises a VH region selected from the group consisting of a VH region as depicted in any one of SEQ ID NO: 34, SEQ ID NO: 43, SEQ ID NO: 52, SEQ ID NO: 61, SEQ ID NO: 70, SEQ ID NO: 79, SEQ ID NO: 88, SEQ ID NO: 97, SEQ ID NO: 106, SEQ ID NO: 115, SEQ ID NO: 118, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, and SEQ ID NO: 245.

More preferably, the antibody construct of the present invention is characterized by the second domain which binds to CD3 on the surface of a T cell comprising a VL region and a VH region selected from the group consisting of:
(a) a VL region as depicted in SEQ ID NO: 35 or 233 and a VH region as depicted in SEQ ID NO: 34 or 236;
(b) a VL region as depicted in SEQ ID NO: 44 or 233 and a VH region as depicted in SEQ ID NO: 43 or 237;
(c) a VL region as depicted in SEQ ID NO: 53 or 233 and a VH region as depicted in SEQ ID NO: 52 or 238;
(d) a VL region as depicted in SEQ ID NO: 62 or 233 and a VH region as depicted in SEQ ID NO: 61 or 239;
(e) a VL region as depicted in SEQ ID NO: 71 or 234 and a VH region as depicted in SEQ ID NO: 70 or 240;
(f) a VL region as depicted in SEQ ID NO: 80 or 233 and a VH region as depicted in SEQ ID NO: 79 or 241;
(g) a VL region as depicted in SEQ ID NO: 89 or 234 and a VH region as depicted in SEQ ID NO: 88 or 242;
(h) a VL region as depicted in SEQ ID NO: 98 or 233 and a VH region as depicted in SEQ ID NO: 97 or 243;
(i) a VL region as depicted in SEQ ID NO: 107 or 235 and a VH region as depicted in SEQ ID NO: 106 or 244;
(j) a VL region as depicted in SEQ ID NO: 116 or 235 and a VH region as depicted in SEQ ID NO: 115 or 245; and
(k) a VL region as depicted in SEQ ID NO: 119 and a VH region as depicted in SEQ ID NO: 118.

A preferred embodiment of the above described antibody construct of the present invention is characterized by the second domain which binds to CD3 on the surface of a T cell comprising an amino acid sequence selected from the group consisting of SEQ ID NOs: 36, 45, 54, 63, 72, 81, 90, 99, 108, 117, 120, 246, 247, 248, 249, 250, 251, 252, 253, 254 and 255.

Amino acid sequence modifications of the antibody constructs described herein are also contemplated. For example, it may be desirable to improve the binding affinity and/or other biological properties of the antibody construct. Amino acid sequence variants of the antibody constructs are prepared by peptide synthesis or by introducing appropriate nucleotide changes into the nucleic acid molecule encoding the antibody constructs. All below described amino acid sequence modifications should result in an antibody construct which retains the desired biological activity of the unmodified parental molecule (such as binding to CLDN18.2 and to CD3, inducing cytotoxicity against CLDN18.2 positive target cells).

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. There are basically four different classes of amino acids determined by different side chains:
(1) non-polar and neutral (uncharged): Ala, Gly, Ile, Leu, Met, Phe, Pro, Val
(2) polar and neutral (uncharged): Asn, Cys (being only slightly polar), Gln, Ser, Thr, Trp (being only slightly polar), Tyr
(3) acidic and polar (negatively charged): Asp and Glu
(4) basic and polar (positively charged): Arg, His, Lys

Hydrophobic amino acids can be divided according to whether they have aliphatic or aromatic side chains. Phe and Trp (being very hydrophobic), Tyr and His (being less hydrophobic) are classified as aromatic amino acids. Strictly speaking, aliphatic means that the side chain contains only hydrogen and carbon atoms. By this strict definition, the amino acids with aliphatic side chains are alanine, isoleucine, leucine (also norleucine), proline and valine. Alanine's side chain, being very short, means that it is not particularly hydrophobic, and proline has an unusual geometry that gives it special roles in proteins. It is often convenient to consider methionine in the same category as isoleucine, leucine and valine, although it also contains a sulphur atom. The unifying theme is that these amino acids contain largely non-reactive and flexible side chains. The amino acids alanine, cysteine, glycine, proline, serine and threonine are often grouped together for the reason that they are all small in size. Gly and Pro may influence chain orientation.

Amino acid modifications include, for example, deletions of residues from, insertions of residues into, and/or substitutions of residues within the amino acid sequences of the antibody constructs. Any combination of deletion, insertion, and/or substitution is made to arrive at a final antibody construct, provided that the final construct possesses the desired characteristics, e.g. the biological activity of the unmodified parental molecule (such as binding to CLDN18.2 and to CD3, inducing cytotoxicity against CLDN18.2 positive target cells). The amino acid changes may also alter post-translational processes of the antibody constructs, such as changing the number or position of glycosylation sites.

For example, 1, 2, 3, 4, 5, or 6 amino acids may be inserted, deleted and/or substituted in each of the CDRs (of course, dependent on their respective length), while 1, 2, 3, 4, 5, 6, 7, 8, 9, 10, 11, 12, 13, 14, 15, 16, 17, 18, 19, 20, or 25 amino acids may be inserted, deleted and/or substituted in each of the FRs. Amino acid sequence insertions also include N-terminal and/or C-terminal additions of amino acids ranging in length from e.g. 1, 2, 3, 4, 5, 6, 7, 8, 9 or 10 residues to polypeptides containing more than 10, e.g. one hundred or more residues, as well as intra-sequence insertions of single or multiple amino acid residues. An insertional variant of the antibody construct of the invention includes the fusion of a polypeptide which increases or extends the serum half-life of the antibody construct to the N-terminus or to the C-terminus of the antibody construct. It is also conceivable that such insertion occurs within the antibody construct, e.g. between the first and the second domain.

The sites of greatest interest for amino acid modifications, in particular for amino acid substitutions, include the hypervariable regions, in particular the individual CDRs of the heavy and/or light chain, but FR alterations in the heavy and/or light chain are also contemplated. The substitutions can be conservative substitutions as described herein. Preferably, 1, 2, 3, 4, 5, 6, 7, 8, 9, or 10 amino acids may be substituted in a CDR, while 1, 2. 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR, respectively. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for the identification of certain residues or regions within the antibody constructs that are preferred locations for mutagenesis is called “alanine scanning mutagenesis” and is described e.g. in Cunningham B.C. and Wells J.A. (Science. 1989 Jun. 2; 244(4908): 1081-5). Here, a residue or group of residues within the antibody construct is/are identified (e.g. charged residues such as Arg, His, Lys, Asp, and Glu) and replaced by a neutral or non-polar amino acid (most preferably alanine or polyalanine) to affect the interaction of the respective amino acid(s) with the epitope of the target protein. Alanine scanning is a technique used to determine the contribution of a specific residue to the stability or function of given protein. Alanine is used because of its non-bulky, chemically inert, methyl functional group that nevertheless mimics the secondary structure preferences that many of the other amino acids possess. Sometimes bulky amino acids such as valine or leucine can be used in cases where conservation of the size of mutated residues is needed. This technique can also be useful to determine whether the side chain of a specific residue plays a significant role in bioactivity. Alanine scanning is usually accomplished by site-directed mutagenesis or randomly by creating a PCR library. Furthermore, computational methods to estimate thermodynamic parameters based on a theoretical alanine substitutions have been developed. The data can be tested by IR, NMR Spectroscopy, mathematical methods, bioassays, etc.

Those amino acid locations demonstrating functional sensitivity to the substitutions (as determined e.g. by alanine scanning) can then be refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done e.g. using assays of antigen (e.g. CLDN18.2 or CD3) binding activity and/or of cytotoxic activity.

Generally, if amino acids are substituted in one or more or all of the CDRs of the heavy and/or light chain, it is envisaged that the then-obtained “substituted” sequence is at least 60% or 65%, more preferably 70% or 75%, even more preferably 80% or 85%, and particularly preferably 90% or 95% identical/homologous to the “original” or “parental” CDR sequence. This means that the degree of identity/homology between the original and the substituted sequence depends on the length of the CDR. For example, a CDR having 5 amino acids in total and comprising one amino acid substitution is 80% identical to the “original” or “parental” CDR sequence, while a CDR having 10 amino acids in total and comprising one amino acid substitution is 90% identical to the “original” or “parental” CDR sequence. Accordingly, the substituted CDRs of the antibody construct of the invention may have different degrees of identity to their original sequences, e.g., CDRL1 may have 80%, while CDRL3 may have 90% of homology. The same considerations apply to the framework regions and to the entire VH and VL regions.

A “variant CDR” is a CDR with a specific sequence homology, similarity, or identity to the parent CDR of the invention, and shares biological function with the parent CDR, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. Generally, the amino acid homology, similarity, or identity between individual variant CDRs is at least 60% to the parent sequences depicted herein, and more typically with increasing homologies, similarities or identities of at least 65% or 70%, preferably at least 75% or 80%, more preferably at least 85%, 90%, 91%, 92%, 93%, 94%, and most preferably 95%, 96%, 97%, 98%, 99%, and almost 100%. The same applies to “variant VH” and “variant VL”. According to one embodiment, the sequence variations within a “variant VH” and/or a “variant VL” do not extend to the CDRs. The present invention is hence directed to an antibody construct as defined herein, comprising VH and VL sequences having a certain sequence homology (see above) to the specific sequences as defined herein (the “parental” VH and VL), wherein the CDR sequences are 100% identical to the specific CDR sequences as defined herein (the “parental” CDRs).

Preferred substitutions (or replacements) are conservative substitutions. However, any substitution (including non-conservative substitutions or one or more from the “exemplary substitutions” listed in Table 1, below) is envisaged, as long as the antibody construct retains its capacity to bind to CLDN18.2 via the first domain and to CD3 or CD3 epsilon via the second domain, and/or provided its CDRs, FRs, VH and/or VL sequences have a degree of identity to the original or parental sequence of at least 60% or 65%, more preferably at least 70% or 75%, even more preferably at least 80% or 85%, and particularly preferably at least 90% or 95%.

A conservative replacement (also called a conservative mutation or a conservative substitution) is an amino acid replacement that changes a given amino acid to a different amino acid with similar biochemical properties (e.g. charge, hydrophobicity, size). Conservative replacements in proteins often have a smaller effect on protein function than non-conservative replacements. Conservative substitutions are shown in Table 1. Exemplary conservative substitutions are shown as “exemplary substitutions”. If such substitutions result in a change in biological activity, then more substantial changes, as further described herein in reference to amino acid classes, may be introduced and the products screened for a desired characteristic.

TABLE 1
Amino acid substitutions

| Original | | Exemplary |
|---|---|---|
| aa | Conservative substitutions | Substitutions |
| Ala (A) | Small aa | Gly, Ser, Thr |
| Arg (R) | Polar aa, in particular Lys | Lys, Gln, Asn |
| Asn (N) | Polar aa, in particular Asp | Asp, Gln, His, Lys, |
| | | Arg |
| Asp (D) | Glu or other polar aa, in particular Asn | Glu, Asn |
| Cys (C) | Small aa | Ser, Ala |
| Gln (Q) | Polar aa, in particular Glu | Glu, Asn |
| Glu (E) | Asp or other polar aa, in particular Gln | Asp, Gln |
| Gly (G) | Small aa, such as Ala | Ala |
| His (H) | | Asn, Gln, Arg, Lys, |
| | | Tyr |
| Ile (I) | Hydrophobic, in particular aliphatic aa | Ala, Val, Met, Leu, |
| | | Phe |
| Leu (L) | Hydrophobic, in particular aliphatic aa | Norleucine, Ile, Ala, |
| | | Val, Met |
| Lys (K) | Polar aa, in particular Arg | Arg, Gln, Asn |
| Met (M) | Hydrophobic, in particular aliphatic aa | Leu, Ala, Ile, Val, |
| | | Phe |
| Phe (F) | Aromatic or hydrophobic aa, in | Tyr, Trp, Leu, Val, |
| | particular Tyr | Ile, Ala |
| Pro (P) | Small aa | Ala |
| Ser (S) | Polar or small aa, in particular Thr | Thr |
| Thr (T) | Polar aa, in particular Ser | Ser |
| Trp (W) | Aromatic aa | Tyr, Phe |
| Tyr (Y) | Aromatic aa, in particular Phe | Phe, Trp, Thr, Ser |
| Val (V) | Hydrophobic, in particular aliphatic aa | Leu, Ile, Ala, Met, |
| | | Phe |

(aa = amino acid)

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Non-conservative substitutions will usually entail exchanging a member of one of the above defined amino acid classes (such as polar, neutral, acidic, basic, aliphatic, aromatic, small . . . ) for another class. Any cysteine residue not involved in maintaining the proper conformation of the antibody construct may be substituted, generally with serine, to improve the oxidative stability of the antibody construct.

Sequence identity, homology and/or similarity of amino acid sequences is determined by using standard techniques known in the art, including, but not limited to, the local sequence identity algorithm of Smith and Waterman, 1981, Adv. Appl. Math. 2:482, the sequence identity alignment algorithm of Needleman and Wunsch (J Mol Biol. 1970 March; 48(3):443-53), the search for similarity method of Pearson and Lipman (Proc Natl Acad Sci USA. 1988 April; 85(8):2444-8), computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (Nucleic Acids Res. 1984 Jan. 11; 12(1 Pt 1):387-95), preferably using the default settings, or by inspection. It is envisaged that percent identity is calculated by FastDB based upon the following parameters: mismatch penalty of 1; gap penalty of 1; gap size penalty of 0.33; and joining penalty of 30. See also “Current Methods in Sequence Comparison and Analysis.” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle (J Mol Evol. 1987; 25(4):351-60); the method is similar to that described by Higgins and Sharp (Comput Appl Biosci. 1989 April; 5(2):151-3). Useful PILEUP parameters include a default gap weight of 3.00, a default gap length weight of 0.10, and weighted end gaps.

Another example of a useful algorithm is the BLAST algorithm, described in: Altschul et al. (J Mol Biol. 1990 Oct. 5; 215(3):403-10.); Altschul et al., (Nucleic Acids Res. 1997 Sep. 1; 25(17):3389-402); and Karlin and Altschul (Proc Natl Acad Sci USA. 1993 Jun. 15; 90(12):5873-7). A particularly useful BLAST program is the WU-Blast-2 program which was obtained from Altschul et al., (Methods Enzymol. 1996; 266:460-80). WU-Blast-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (Nucleic Acids Res. 1997 Sep. 1; 25(17):3389-402). Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

In line herewith, the term “percent (%) nucleic acid sequence identity/homology/similarity” with respect to the nucleic acid sequence encoding the antibody constructs identified herein is defined as the percentage of nucleotide residues in a candidate sequence that are identical with the nucleotide residues in the coding sequence of the antibody construct. One method to align two sequences and thereby determine their homology uses the BLASTN module of WU-Blast2 set to the default parameters, with overlap span and overlap fraction set to 1 and 0.125, respectively. Generally, the nucleic acid sequence homology, similarity, or identity between the nucleotide sequences encoding individual variant CDRs and the nucleotide sequences depicted herein are at least 60%, and more typically with increasing homologies, similarities or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Again, the same applies to nucleic acid sequence encoding the “variant VH” and/or “variant VL”.

In one embodiment, the percentage of identity to human germline of the antibody constructs according to the invention, or of the first and second domain (binding domains) of these antibody constructs, is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang W. Y. and Foote J. (Methods. 2005 May; 36(1):3-10) demonstrate that the reduction of non-human portions of drug antibody constructs leads to a decrease of risk of inducing anti-drug antibodies in the patients during treatment. By comparing an exhaustive number of clinically evaluated antibody drugs and the respective immunogenicity data, the trend is shown that humanization of the variable regions of antibodies/antibody constructs makes the protein less immunogenic (average 5.1% of patients) than antibodies/antibody constructs carrying unaltered non-human variable regions (average 23.59% of patients). A higher degree of identity to human sequences is hence desirable for protein therapeutics based on variable regions and in the form of antibody constructs. For the purpose of determining the germline identity, the V-regions of VL can be aligned with the amino acid sequences of human germline V segments and J segments (http://www2.mrc-lmb.cam.ac.uk/vbase/) using Vector NTI software and the amino acid sequence calculated by dividing the identical amino acid residues by the total number of amino acid residues of the VL in percent. The same can be done for the VH segments (http://www2.mrc-lmb.cam.ac.uk/vbase/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the antibody constructs of the present invention exhibit high monomer yields under standard research scale conditions, e.g., in a standard two-step purification process. It is envisaged that the monomer yield of the antibody constructs according to the invention is ≥0.25 mg/L supernatant (SN), preferably ≥0.5 mg/L SN, more preferably ≥1 mg/L SN, even more preferably ≥2 mg/L SN and most preferably ≥3 mg/L SN. The yield of the antibody construct denominated “CL-1×I2C-6His” was shown to be 4.1 mg/L supernatant, and the yield of the antibody construct denominated “CL-1×I2C-scFc” was shown to be 36.5 mg/L supernatant.

Likewise, the yield of the dimeric antibody construct isoforms and hence the monomer percentage (i.e., monomer:(monomer+dimer)) of the antibody constructs can be determined. The productivity of monomeric and dimeric antibody constructs and the calculated monomer percentage can e.g. be obtained in the SEC purification step of culture supernatant from standardized research-scale production in roller bottles. According to one embodiment, the monomer percentage of the antibody constructs of the invention is ≥80%, more preferably ≥85%, even more preferably ≥90%, and most preferably ≥95%.

According to one embodiment, the antibody constructs of the invention have a plasma stability (ratio of EC₅₀ with plasma to EC₅₀ w/o plasma) of ≤5 or ≤4, more preferably ≤3.5 or ≤3, even more preferably ≤2.5 or ≤2, and most preferably ≤1.5 or ≤1. The plasma stability of an antibody construct can be tested by incubation of the purified construct in human plasma at 37ºC for 24 to 96 hours, e.g. at a concentration of 2-20 μg/ml, followed by EC₅₀ determination in an 18 h 51-chromium release or in a 48 h FACS cytotoxicity assay (assays e.g. as described in Examples 7.1 and 7.2). The effector cells in the cytotoxicity assay can be stimulated enriched human CD8 positive T cells (preferred) or unstimulated human PBMC. Target cells can e.g. be CHO cells transfected with human CLDN18.2. The effector to target cell (E:T) ratio can be 10:1. The starting concentration of the antibody constructs in the cytotoxicity assay can be 0.01-0.1 μg/ml. The human plasma pool used for this purpose is derived from the blood of healthy donors collected by EDTA coated syringes. Cellular components are removed by centrifugation and the upper plasma phase is collected and subsequently pooled. As control, non-incubated antibody constructs are diluted immediately prior to the cytotoxicity assay in appropriate medium such as RPMI-1640. The plasma stability is calculated as ratio of EC₅₀ (after plasma incubation) to EC₅₀ (control/no incubation).

It is furthermore envisaged that the monomer to dimer conversion of the antibody constructs of the invention is low. The conversion can be measured under different conditions and analyzed by high performance size exclusion chromatography. See Example 8. For example, incubation of the monomeric isoforms of the antibody constructs can be carried out for 7 days at 37° C. in generic formulation buffer and at concentrations of e.g. 100 μg/ml or 250 μg/ml in an incubator, followed by high performance SEC to determine the percentage of initially monomeric antibody construct which had been converted into dimeric antibody construct. Under these conditions, it is envisaged that the antibody constructs of the invention show a dimer percentage that is ≤8%, preferably ≤6%, more preferably ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is likewise envisaged that the antibody constructs of the present invention present with very low dimer conversion after several freeze/thaw cycles. For example, the antibody construct monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antibody construct which had been converted into dimeric antibody construct. It is envisaged that the dimer percentages of the antibody constructs are ≤8%, preferably ≤6%, more preferably ≤5%, more preferably ≤4%, even more preferably ≤3%, even more preferably ≤2.5%, even more preferably ≤2%, even more preferably ≤1.5%, and most preferably ≤1% or ≤0.5% or even 0%, for example after three freeze/thaw cycles.

According to one embodiment, the antibody constructs of the present invention show a favorable thermostability with aggregation temperatures ≥45° C. or ≥46° C., more preferably ≥47° C. or ≥48° C., even more preferably ≥49° ° C. or ≥50° ° C., and most preferably ≥51° C. The thermostability parameter can be determined in terms of antibody aggregation temperature as follows: Antibody solution at a concentration 250 μg/ml is transferred into a single use cuvette and placed in a dynamic light scattering (DLS) device. The sample is heated from 40° ° C. to 70° C. at a heating rate of 0.5° C./min with constant acquisition of the measured radius. Increase of radius indicating melting of the protein and aggregation is used to calculate the aggregation temperature of the antibody. See Example 9.

Alternatively, temperature melting curves can be determined by differential scanning calorimetry (DSC) to determine intrinsic biophysical protein stabilities of the antibody constructs. These experiments can be performed using a MicroCal LLC VP-DSC device. The energy uptake of a sample containing an antibody construct is recorded from 20° ° C. to 90° ° C. compared to a sample containing only the formulation buffer. The antibody constructs are adjusted to a final concentration of 250 μg/ml e.g. in SEC running buffer. For recording of the respective melting curve, the overall sample temperature is increased stepwise. Energy uptake of the sample and the formulation buffer reference is recorded at each temperature. The difference in energy uptake Cp (kcal/mole/° C.) of the sample minus the reference is plotted against the respective temperature. The melting temperature is defined as the temperature at the first maximum of energy uptake.

The antibody constructs of the invention are also envisaged to have a turbidity of ≤0.2 or ≤0.15, preferably of ≤0.10 or ≤0.08, more preferably of ≤0.06 or ≤0.05, and most preferably of ≤0.04 or ≤0.03. The turbidity can be measured by OD340 at a concentration of the antibody construct of 2.5 mg/ml and 16 h incubation at 5° C. See Example 10.

Changes in the potency of a target x CD3 antibody construct as a function of preincubation of the construct on the target cells in the absence of T cells can be measured. If an antibody construct is internalized, it is expected to undergo lysosomal degradation. The effective concentration is hence expected to decrease over time, and thus the apparent potency should decrease as well. The effect has been observed with some targets, for which this is a known phenomenon. Antibody constructs of the invention are envisaged to not be internalized or to not undergo significant internalization by the target cell. The rate of internalization can be assayed e.g. as described in the following: T cells are counted and diluted to a concentration of 1×10⁵/ml in assay media. Target positive target cells are counted and plated e.g. at 2500 cells per well (cpw). The antibody construct is diluted serially 1:2, e.g. at a starting concentration of 100 nM. The antibody construct is added to the culture assay plates to allow for 0 hours, 1 hour or 2 hours of incubation prior to addition of the T cells. Then the T cells are plated at 25000 cpw (E:T=10:1), and the assay is incubated for 48 hours at 37° C. Target cell survival is analyzed e.g. with the Steady-Glo® system (25 μl/well). Preferably, the internalization rate (e.g. measured as a decrease in cytotoxicity) is ≤20% after a 2-hour (pre-)incubation of the antibody construct with the target cell, more preferably ≤15%, even more preferably ≤10%, and most preferably ≤5%.

It is furthermore envisaged for an antibody construct of the invention that shed or soluble target does not significantly impair its efficacy or biologic activity. This can be measured e.g. in a cytotoxicity assay where soluble target is added at increasing concentrations to the assay, e.g. at 0 nM-0.3 nM-0.7 nM-1 nM-3 nM-7 nM-12 nM. An exemplary E:T value is 10:1. The EC50 value of the tested antibody construct should not be significantly increased in the presence of soluble target.

In a further embodiment, the antibody construct according to the invention is stable at acidic pH. The more tolerant the antibody construct behaves at unphysiologic pH such as pH 5.5 (a pH which is required to run e.g. a cation exchange chromatography), the higher is the recovery of the antibody construct eluted from an ion exchange column relative to the total amount of loaded protein. Recovery of the antibody construct from an ion (e.g., cation) exchange column at pH 5.5 is preferably ≥30%, more preferably ≥40%, more preferably ≥50%, even more preferably ≥60%, even more preferably ≥70%, even more preferably ≥80%, and most preferably ≥95%. The percentage represents the area under the curve (=AUC) of the main peak. See Example 11.

It is furthermore envisaged that the antibody constructs of the present invention exhibit therapeutic efficacy, which manifests as anti-tumor activity or tumor growth inhibition. This can e.g. be assessed in a study as disclosed in Example 13 or 14. In one embodiment, the tumor growth inhibition of the antibody construct of the invention T/C [%] is ≤70, ≤60, ≤50, ≤40, ≤30, ≤20, ≤10, ≤5, ≤4, ≤3, or ≤2. Modification or adjustment of certain parameters of these studies (such as the number of injected tumor cells, the site of injection, the number of transplanted human T cells, the amount of antibody constructs to be administered, and the timelines) is also envisaged, while still arriving at a meaningful and reproducible result.

The invention further provides a polynucleotide/nucleic acid molecule encoding an antibody construct of the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide of the present invention can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule of the invention, capable of expressing the antibody construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, more often than not, this difference is in the second or third position. For instance, codons GAA and GAG both specify glutamic acid and exhibit redundancy; but, neither specifies any other amino acid and thus demonstrate no ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. in order to increase protein expression. It is hence envisaged that the polynucleotides/nucleic acid molecules of the present disclosure are codon optimized. Nevertheless, the polynucleotide/nucleic acid molecule encoding an antibody construct of the invention may be designed using any codon that encodes the desired amino acid.

According to one embodiment, the polynucleotide/nucleic acid molecule of the present invention encoding the antibody construct of the invention is in the form of one single molecule or in the form of two or more separate molecules. If the antibody construct of the present invention is a single chain antibody construct, the polynucleotide/nucleic acid molecule encoding such construct will most likely also be in the form of one single molecule. However, it is also envisaged that different components of the antibody construct (such as the different domains, e.g. the domain which binds to CLDN18.2, the domain which binds to CD3, and/or further domains such as antibody constant domains) are located on separate polypeptide chains, in which case the polynucleotide/nucleic acid molecule is most likely in the form of two or more separate molecules.

The same applies for the vector comprising a polynucleotide/nucleic acid molecule of the present invention. If the antibody construct of the present invention is a single chain antibody construct, one vector may comprise the polynucleotide which encodes the antibody construct in one single location (as one single open reading frame, ORF). One vector may also comprise two or more polynucleotides/nucleic acid molecules at separate locations (with individual ORFs), each one of them encoding a different component of the antibody construct of the invention. It is envisaged that the vector comprising the polynucleotide/nucleic acid molecule of the present invention is in the form of one single vector or two or more separate vectors. In one embodiment, and for the purpose of expressing the antibody construct in a host cell, the host cell of the invention should comprise the polynucleotide/nucleic acid molecule encoding the antibody construct or the vector comprising such polynucleotide/nucleic acid molecule in their entirety, meaning that all components of the antibody construct—whether encoded as one single molecule or in separate molecules/locations—will assemble after translation and form together the biologically active antibody construct of the invention.

The invention also provides a vector comprising a polynucleotide/nucleic acid molecule of the invention. A vector is a nucleic acid molecule used as a vehicle to transfer (foreign) genetic material into a cell, usually for the purpose of replication and/or expression. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density, and can also be artificially induced.

Moreover, the invention provides a host cell transformed or transfected with the polynucleotide/nucleic acid molecule of the invention or with the vector of the invention.

As used herein, the terms “host cell” or “recipient cell” are intended to include any individual cell or cell culture that can be or has been recipient of vectors, exogenous nucleic acid molecules and/or polynucleotides encoding the antibody construct of the present invention; and/or recipients of the antibody construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include—but are not limited to—bacteria (such as E. coli), yeast cells, fungi cells, plant cells, and animal cells such as insect cells and mammalian cells, e.g., hamster, murine, rat, macaque or human.

In addition to prokaryotes, eukaryotic microbes such as filamentous fungi or yeast are suitable cloning or expression hosts for the antibody construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated antibody construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

In a further embodiment, the invention provides a process for producing an antibody construct of the invention, said process comprising culturing a host cell of the invention under conditions allowing the expression of the antibody construct of the invention and recovering the produced antibody construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a CO2 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures so they can be grown to a higher density than adherent conditions would allow.

The term “expression” includes any step involved in the production of an antibody construct of the invention including, but not limited to, transcription, post-transcriptional modification, translation, folding, post-translational modification, targeting to specific subcellular or extracellular locations, and secretion. The term “recovering” refers to a series of processes intended to isolate the antibody construct from the cell culture. The “recovering” or “purification” process may separate the protein and non-protein parts of the cell culture, and finally separate the desired antibody construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the antibody construct can be produced intracellularly, in the periplasmic space, or directly secreted into the medium. If the antibody construct is produced intracellularly, as a first step, the particulate debris, either host cells or lysed fragments, are removed, for example, by centrifugation or ultrafiltration. The antibody construct of the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner similar to the process for purifying an antibody construct expressed in mammalian cells and secreted into the medium. Carter et al. (Biotechnology (NY) 1992 February; 10(2):163-7) describe a procedure for isolating antibodies which are secreted to the periplasmic space of E. coli.

Where the antibody is secreted into the medium, supernatants from such expression systems are generally first concentrated using a commercially available protein concentration filter, for example, an ultrafiltration unit.

The antibody construct of the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the antibody construct to be recovered.

A protease inhibitor may be included in any of the foregoing steps to inhibit proteolysis, and antibiotics may be included to prevent the growth of contaminants.

Moreover, the invention provides a pharmaceutical composition or formulation comprising an antibody construct of the invention or an antibody construct produced according to the process of the invention.

As used herein, the term “pharmaceutical composition” relates to a composition which is suitable for administration to a patient, preferably a human patient. The particularly preferred pharmaceutical composition of this invention comprises one or a plurality of the antibody construct(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antibody construct of the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may contain formulation materials for the purpose of modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company). In such embodiments, suitable formulation materials may include, but are not limited to:
amino acids
antimicrobials such as antibacterial and antifungal agents
antioxidants
buffers, buffer systems and buffering agents which are used to maintain the composition at physiological pH or at a slightly lower pH, typically within a pH range of from about 5 to about 8 or 9
non-aqueous solvents, vegetable oils, and injectable organic esters
aqueous carriers including water, alcoholic/aqueous solutions, emulsions or suspensions, including saline and buffered media
biodegradable polymers such as polyesters
bulking agents
chelating agents
isotonic and absorption delaying agents
complexing agents
fillers
carbohydrates
(low molecular weight) proteins, polypeptides or proteinaceous carriers, preferably of human origin
coloring and flavouring agents
sulfur containing reducing agents
diluting agents
emulsifying agents
hydrophilic polymers
salt-forming counter-ions
preservatives
metal complexes
solvents and co-solvents
sugars and sugar alcohols
suspending agents
surfactants or wetting agents
stability enhancing agents
tonicity enhancing agents
parenteral delivery vehicles
intravenous delivery vehicles

It is common knowledge that the different constituents of the pharmaceutical composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In the context of the present invention, a pharmaceutical composition may comprise:
(a) an antibody construct as described herein,
(b) at least one buffer agent,
(c) at least one saccharide, and
(d) at least one surfactant;
wherein the pH of the pharmaceutical composition is in the range of 3.5 to 6.

In the composition described above, the first domain preferably has an isoelectric point (pI) in the range of 4 to 9,5; the second domain has a pI in the range of 8 to 10, preferably 8.5 to 9.0; and the antibody construct optionally comprises a third domain comprising two polypeptide monomers, each comprising a hinge, a CH2 domain and a CH3 domain, wherein said two polypeptide monomers are fused to each other via a peptide linker;

In the composition described above, it is further envisaged that the at least one buffer agent is present at a concentration range of 5 to 200 mM, more preferably at a concentration range of 10 to 50 mM. It is also envisaged that the at least one saccharide is selected from the group consisting of monosaccharide, disaccharide, cyclic polysaccharide, sugar alcohol, linear branched dextran or linear non-branched dextran. It is also envisaged that the disacchade is selected from the group consisting of sucrose, trehalose and mannitol, sorbitol, and combinations thereof. It is further envisaged that the sugar alcohol is sorbitol. It is also envisaged that the at least one saccharide is present at a concentration in the range of 1 to 15% (m/V), preferably in a concentration range of 9 to 12% (m/V). It is further envisaged that the antibody construct is present in a concentration range of 0.1 to 8 mg/ml, preferably of 0.2-2.5 mg/ml, more preferably of 0.25-1.0 mg/ml.

According to one embodiment of the composition described above, the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged that the at least one surfactant is present at a concentration in the range of 0.004 to 0.5% (m/V), preferably in the range of 0.001 to 0.01% (m/V). It is envisaged that the pH of the composition is in the range of 4.0 to 5.0, preferably 4.2. It is also envisaged that the pharmaceutical composition has an osmolarity in the range of 150 to 500 mOsm. It is further envisaged that the pharmaceutical composition further comprises an excipient selected from the group consisting of one or more polyol(s) and one or more amino acid(s). It is envisaged in the context of the present invention that said one or more excipient is present in the concentration range of 0.1 to 15% (w/V).

The present invention also provides a pharmaceutical composition comprising
(a) the antibody construct as described herein, preferably in a concentration range of 0.1 to 8 mg/ml, preferably of 0.2-2.5 mg/ml, more preferably of 0.25-1.0 mg/ml;
(b) 10 mM glutamate or acetate;
(c) 9% (m/V) sucrose or 6% (m/V) sucrose and 6% (m/V) hydroxypropyl-β-cyclodextrin;
(d) 0.01% (m/V) polysorbate 80;
wherein the pH of the liquid pharmaceutical composition is 4.2.

It is envisaged that the composition of the invention might comprise, in addition to the antibody construct of the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions, drugs modulating the inflammatory response, drugs acting on the circulatory system and/or agents such as cytokines known in the art. It is also envisaged that the antibody construct of the present invention is applied in a co-therapy, i.e., in combination with another anti-cancer medicament.

In this context, it is envisaged that the pharmaceutical composition of the invention (which comprises an antibody construct comprising a first domain which binds to CLDN18.2 on the surface of a target cell and a second domain which binds to CD3 on the surface of a T cell, as described in more detail herein above) furthermore comprises an agent, preferably an antibody or antibody construct, which binds to a protein of the immune checkpoint pathway (such as PD-1 or CTLA-4) or to a co-stimulatory immune checkpoint receptor (such as 4-1BB). The present invention also refers to a combination of an antibody construct according to the invention (which comprises an antibody construct comprising a first domain which binds to CLDN18.2 on the surface of a target cell and a second domain which binds to CD3 on the surface of a T cell, as described in more detail herein above) and an agent, preferably an antibody or antibody construct, which binds to a protein of the immune checkpoint pathway (such as PD-1 or CTLA-4) or to a co-stimulatory immune checkpoint receptor (such as 4-1BB). Due to the nature of the at least two ingredients of the combination, namely their pharmaceutical activity, the combination can also be referred to as a therapeutic combination. In some embodiments, the combination can be in the form of a pharmaceutical composition or of a kit. According to one embodiment, the pharmaceutical composition or the combination comprises an antibody construct of the invention and an antibody or antibody construct which binds to PD-1. Anti-PD-1 binding proteins useful for this purpose are e.g. described in detail in PCT/US2019/013205.

Hence, in a further aspect, the present invention is directed to a pharmaceutical composition or to a combination comprising:
an antibody construct comprising a first domain which binds to CLDN18.2 on the surface of a target cell and a second domain which binds to CD3 on the surface of a T cell, as described in more detail herein above, and
an antibody or antibody construct which binds to PD-1 and comprises:
   a) a VH region comprising CDR-H1 as depicted in SEQ ID NO: 292, CDR-H2 as depicted in SEQ ID NO: 293, and CDR-H3 as depicted in SEQ ID NO: 294, and/or a VL region comprising CDR-L1 as depicted in SEQ ID NO: 295, CDR-L2 as depicted in SEQ ID NO: 296 and CDR-L3 as depicted in SEQ ID NO: 297;
   b) a VH region comprising CDR-H1 as depicted in SEQ ID NO: 302, CDR-H2 as depicted in SEQ ID NO: 303, and CDR-H3 as depicted in SEQ ID NO: 304, and/or a VL region comprising CDR-L1 as depicted in SEQ ID NO: 305, CDR-L2 as depicted in SEQ ID NO: 306 and CDR-L3 as depicted in SEQ ID NO: 307;
   c) a VH region comprising CDR-H1 as depicted in SEQ ID NO: 312, CDR-H2 as depicted in SEQ ID NO: 313, and CDR-H3 as depicted in SEQ ID NO: 314, and/or a VL region comprising CDR-L1 as depicted in SEQ ID NO: 315, CDR-L2 as depicted in SEQ ID NO: 316 and CDR-L3 as depicted in SEQ ID NO: 317;
   d) a VH region comprising CDR-H1 as depicted in SEQ ID NO: 322, CDR-H2 as depicted in SEQ ID NO: 323, and CDR-H3 as depicted in SEQ ID NO: 324, and/or a VL region comprising CDR-L1 as depicted in SEQ ID NO: 325, CDR-L2 as depicted in SEQ ID NO: 326 and CDR-L3 as depicted in SEQ ID NO: 327;
   e) a VH region comprising CDR-H1 as depicted in SEQ ID NO: 332, CDR-H2 as depicted in SEQ ID NO: 333, and CDR-H3 as depicted in SEQ ID NO: 334, and/or a VL region comprising CDR-L1 as depicted in SEQ ID NO: 335, CDR-L2 as depicted in SEQ ID NO: 336 and CDR-L3 as depicted in SEQ ID NO: 337;
   f) a VH region comprising CDR-H1 as depicted in SEQ ID NO: 342, CDR-H2 as depicted in SEQ ID NO: 343, and CDR-H3 as depicted in SEQ ID NO: 344, and/or a VL region comprising CDR-L1 as depicted in SEQ ID NO: 345, CDR-L2 as depicted in SEQ ID NO: 346 and CDR-L3 as depicted in SEQ ID NO: 347;
   g) a VH region comprising CDR-H1 as depicted in SEQ ID NO: 352, CDR-H2 as depicted in SEQ ID NO: 353, and CDR-H3 as depicted in SEQ ID NO: 354, and/or a VL region comprising CDR-L1 as depicted in SEQ ID NO: 355, CDR-L2 as depicted in SEQ ID NO: 356 and CDR-L3 as depicted in SEQ ID NO: 357; or
   h) a VH region comprising CDR-H1 as depicted in SEQ ID NO: 362, CDR-H2 as depicted in SEQ ID NO: 363, and CDR-H3 as depicted in SEQ ID NO: 364, and/or a VL region comprising CDR-L1 as depicted in SEQ ID NO: 365, CDR-L2 as depicted in SEQ ID NO: 366 and CDR-L3 as depicted in SEQ ID NO: 367.

In one embodiment, the above described antibody or antibody construct which binds to PD-1 comprises
a) a VH region as depicted in SEQ ID NO: 298, and a VL region as depicted in SEQ ID NO: 299;
b) a VH region as depicted in SEQ ID NO: 308, and a VL region as depicted in SEQ ID NO: 309;
c) a VH region as depicted in SEQ ID NO: 318, and a VL region as depicted in SEQ ID NO: 319;
d) a VH region as depicted in SEQ ID NO: 328, and a VL region as depicted in SEQ ID NO: 329;
e) a VH region as depicted in SEQ ID NO: 338, and a VL region as depicted in SEQ ID NO: 339;
f) a VH region as depicted in SEQ ID NO: 348, and a VL region as depicted in SEQ ID NO: 349;
g) a VH region as depicted in SEQ ID NO: 358, and a VL region as depicted in SEQ ID NO: 359; or
h) a VH region as depicted in SEQ ID NO: 368, and a VL region as depicted in SEQ ID NO: 369.

In one embodiment, the above antibody or antibody construct which binds to PD-1 comprises:
a) a heavy chain as depicted in SEQ ID NO: 300, and a light chain as depicted in SEQ ID NO: 301;
b) a heavy chain as depicted in SEQ ID NO: 310, and a light chain as depicted in SEQ ID NO: 311;
c) a heavy chain as depicted in SEQ ID NO: 320, and a light chain as depicted in SEQ ID NO: 321;
d) a heavy chain as depicted in SEQ ID NO: 330, and a light chain as depicted in SEQ ID NO: 331;
e) a heavy chain as depicted in SEQ ID NO: 340, and a light chain as depicted in SEQ ID NO: 341;
f) a heavy chain as depicted in SEQ ID NO: 350, and a light chain as depicted in SEQ ID NO: 351;
g) a heavy chain as depicted in SEQ ID NO: 360, and a light chain as depicted in SEQ ID NO: 361; or
h) a heavy chain as depicted in SEQ ID NO: 370, and a light chain as depicted in SEQ ID NO: 371.

In certain embodiments, the optimal pharmaceutical composition is determined depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibody construct of the invention. In certain embodiments, the primary vehicle or carrier in a pharmaceutical composition may be either aqueous or non-aqueous in nature. For example, a suitable vehicle or carrier may be water for injection or physiological saline solution, possibly supplemented with other materials common in compositions for parenteral administration. In certain embodiments, the compositions comprising the antibody construct of the invention may be prepared for storage by mixing the selected composition having the desired degree of purity with optional formulation agents (Remington's Pharmaceutical Sciences, supra) in the form of a lyophilized cake or an aqueous solution. Further, in certain embodiments, the antibody construct of the invention may be formulated as a lyophilizate using appropriate excipients.

When parenteral administration is contemplated, the therapeutic compositions for use in this invention may be provided in the form of a pyrogen-free, parenterally acceptable aqueous solution comprising the desired antibody construct of the invention in a pharmaceutically acceptable vehicle. A particularly suitable vehicle for parenteral injection is sterile distilled water in which the antibody construct of the invention is formulated as a sterile, isotonic solution, properly preserved. In certain embodiments, the preparation can involve the formulation of the desired molecule with an agent that may provide controlled or sustained release of the product which can be delivered via depot injection, or that may promote sustained duration in the circulation. In certain embodiments, implantable drug delivery devices may be used to introduce the desired antibody construct.

Additional pharmaceutical compositions will be evident to those skilled in the art, including formulations involving the antibody construct of the invention in sustained or controlled delivery formulations. Techniques for formulating a variety of sustained- or controlled-delivery means are known to those skilled in the art. The antibody construct may also be entrapped in microcapsules prepared, for example, by coacervation techniques or by interfacial polymerization, in colloidal drug delivery systems, or in macroemulsions. Such techniques are disclosed in Remington's Pharmaceutical Sciences, supra.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions are generally placed into a container having a sterile access port, for example, an intravenous solution bag or vial having a stopper pierceable by a hypodermic injection needle.

Another aspect of the invention includes self-buffering formulations comprising the antibody construct of the invention, which can be used as pharmaceutical compositions, as described in international patent application WO 2006/138181. A variety of publications are available on protein stabilization and formulation materials and methods useful in this regard, such as Arawaka T. et al., Pharm Res. 1991 March; 8(3):285-91; Kendrick et al., “Physical stabilization of proteins in aqueous solution” in: Rational Design of Stable Protein Formulations: Theory and Practice, Carpenter and Manning, eds. Pharmaceutical Biotechnology. 13: 61-84 (2002), and Randolph and Jones, Pharm Biotechnol. 2002; 13:159-75, see particularly the parts pertinent to excipients and processes for self-buffering protein formulations, especially as to protein pharmaceutical products and processes for veterinary and/or human medical uses.

Salts may be used in accordance with certain embodiments of the invention, e.g. in order to adjust the ionic strength and/or the isotonicity of a composition or formulation and/or to improve the solubility and/or physical stability of an antibody construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the antibody construct of the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the antibody construct of the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption. Surfactants also are commonly used to control protein conformational stability. The use of surfactants in this regard is protein specific, since one specific surfactant will typically stabilize some proteins and destabilize others.

Certain embodiments of the formulation or composition comprising the antibody construct of the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants for use in therapeutic protein formulations in accordance with the present invention can be water-soluble and maintain their activity throughout the shelf life of the product (the composition comprising the antibody construct). Antioxidants can also damage proteins and should hence—among other things—be selected in a way to eliminate or sufficiently reduce the possibility of antioxidants damaging the antibody construct or other proteins in the formulation.

Certain embodiments of the formulation or composition comprising the antibody construct of the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the antibody construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of formulation of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to, white blood cell counts, differentials, fluorescence activated cell sorting, bone marrow aspiration. In addition, various disease specific clinical chemistry parameters and other established standard methods may be used. Furthermore, computer-aided tomography, X-ray, nuclear magnetic resonance tomography, positron-emission tomography scanning, lymph node biopsies/histologies and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance (e.g., an antibody construct of the invention) in blood plasma/serum to reach one-half of its steady-state value (“serum half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver. “Volume of distribution” (V_{D}) means the degree to which a drug is distributed in body tissue rather than the blood plasma, a higher V_{D} indicating a greater amount of tissue distribution. The retention of a drug can occur throughout the various compartments of the body, such as intracellular and extracellular spaces, tissues and organs, etc. “Degree of blood serum binding” means the propensity of a drug to interact with and bind to blood serum proteins, such as albumin, leading to a reduction or loss of biological activity of the drug.

Pharmacokinetic parameters also include bioavailability, lag time (T lag), Tmax, absorption rates, and/or Cmax for a given amount of drug administered. “Bioavailability” refers to the fraction of an administered dose of a drug/substance that reaches the systemic circulation (the blood compartment). When a medication is administered intravenously, its bioavailability is considered to be 100%. However, when a medication is administered via other routes (such as orally), its bioavailability generally decreases. “Lag time” means the time delay between the administration of the drug and its detection and measurability in blood or plasma. Cmax is the maximum plasma concentration that a drug achieves after its administration (and before the administration of a second dose). Tmax is the time at which Cmax is reached. The time to reach a blood or tissue concentration of the drug which is required for its biological effect is influenced by all parameters. Pharmacokinetic parameters of antibody constructs exhibiting cross-species specificity may be determined in preclinical animal testing in non-chimpanzee primates as outlined above and set forth e.g. in Schlereth et al. (supra).

One embodiment provides the antibody construct of the invention (or the antibody construct produced according to the process of the invention) for use in the prevention, treatment or amelioration of a disease, preferably a neoplasm. Another embodiment provides the use of the antibody construct of the invention (or of the antibody construct produced according to the process of the invention) in the manufacture of a medicament for the prevention, treatment or amelioration of a disease, preferably a neoplasm. It is also envisaged to provide a method for the prevention, treatment or amelioration of a disease, preferably a neoplasm, comprising the step of administering to a subject in need thereof the antibody construct of the present invention (or the antibody construct produced according to the process of the present invention). The terms “subject in need”, “patient” or those “in need of treatment” include those already with the disease, as well as those in which the disease is to be prevented. The terms also include human and other mammalian subjects that receive either prophylactic or therapeutic treatment.

The antibody constructs of the invention and the formulations/pharmaceutical compositions described herein are useful in the treatment, amelioration and/or prevention of the medical condition as described herein in a patient in need thereof. The term “treatment” refers to both therapeutic treatment and prophylactic or preventative measures. Treatment includes the application or administration of the antibody constructs/pharmaceutical composition to the body, to an isolated tissue, or to a cell from a patient or a subject in need who has a disease/disorder as described herein, a symptom of such disease/disorder, or a predisposition toward such disease/disorder, with the purpose to cure, heal, alleviate, relieve, alter, remedy, ameliorate, improve, or affect the disease, the symptom of the disease, or the predisposition toward the disease. The term “amelioration” as used herein refers to any improvement of the disease state of a patient, by the administration of an antibody construct according to the invention to such patient or subject in need thereof. Such an improvement may be seen as a slowing or stopping of the progression of the disease of the patient, and/or as a decrease in severity of disease symptoms, an increase in frequency or duration of disease symptom-free periods or a prevention of impairment or disability due to the disease. The term “prevention” as used herein means the avoidance of the occurrence or of the re-occurrence of a disease as specified herein, by the administration of an antibody construct according to the invention to a subject in need thereof.

The term “disease” refers to any condition that would benefit from treatment with the antibody construct or the pharmaceutical composition described herein. This includes chronic and acute disorders or diseases including those pathological conditions that predispose the mammal to the disease in question. The disease is preferably a neoplasm, cancer or tumor. The disease, neoplasm, cancer or tumor is preferably CLDN18.2 positive, i.e. it is characterized by expression or overexpression of CLDN18.2.

A “neoplasm” is an abnormal growth of tissue, usually but not always forming a mass. When also forming a mass, it is commonly referred to as a “tumor”. In brain tumors, the uncontrolled division of cells means that the mass of a neoplasm increases in size, and in a confined space such as the intracranial cavity this quickly becomes problematic because the mass invades the space of the brain pushing it aside, leading to compression of the brain tissue and increased intracranial pressure and destruction of brain parenchyma. Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. For example, a brain tumor occurs when abnormal cells form within the brain. Most cancers develop at their primary site but then go on to metastasize or spread to other parts (e.g. tissues and organs) of the body. These further tumors are “secondary tumors”. Most cancers continue to be called after their primary site, even after they have spread to other parts of the body.

Lymphomas and leukemias are lymphoid neoplasms. For the purposes of the present invention, they are also encompassed by the terms “tumor” or “cancer”. For the purposes of the present invention, the terms “neoplasm”, “tumor” and “cancer” may be used interchangeably, and they comprise both primary tumors/cancers and secondary tumors/cancers (or “metastases”), as well as mass-forming neoplasms (tumors) and lymphoid neoplasms (such as lymphomas and leukemias), and also MRD.

The term “minimal residual disease” (MRD) refers to the evidence for the presence of small numbers of residual cancer cells that remain in the patient after cancer treatment, e.g. when the patient is in remission (no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a cancer are envisaged to also encompass “prevention, treatment or amelioration of MRD”, whether the MRD was detected or not.

In one embodiment of the invention, the neoplasm, cancer or tumor is selected from the group including, but not limited to, (or consisting of) gastrointestinal cancer, ovarian cancer and lung cancer. According to one embodiment of the invention, the gastrointestinal cancer is selected from the group consisting of gastric or stomach cancer, esophageal cancer, gastroesophageal cancer, pancreatic cancer, and colorectal cancer. According to another embodiment of the invention, the ovarian cancer is mucinous ovarian cancer. According to a further embodiment of the invention, the lung cancer is non-small cell lung cancer.

The antibody construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration. Formulations and compositions thus may be designed in accordance with the invention for delivery by any suitable route of administration. In the context of the present invention, the routes of administration include, but are not limited to topical routes, enteral routes and parenteral routes.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical compositions and the antibody construct of this invention are particularly useful for parenteral administration, e.g., intravenous delivery, for example by injection or infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

The compositions of the present invention can be administered to the subject at a suitable dose which can be determined e.g. in dose escalating studies. As set forth above, the antibody construct of the invention exhibiting cross-species specificity as described herein can also be advantageously used in in preclinical testing in non-chimpanzee primates. The dosage regimen will be determined by the attending physician and clinical factors. As is well known in the medical art, dosages for any one patient depend upon many factors, including the patient's size, body surface area, age, the particular compound to be administered, sex, time and route of administration, general health, and other drugs being administered concurrently.

An “effective dose” is an amount of a therapeutic agent sufficient to achieve or at least partially achieve a desired effect. A “therapeutically effective dose” is an amount sufficient to cure or at least partially arrest the disease and its complications, signs and symptoms in a patient suffering from the disease. Amounts or doses effective for this use will depend on the disease to be treated (the indication), the delivered antibody construct, the therapeutic context and objectives, the severity of the disease, prior therapy, the patient's clinical history and response to the therapeutic agent, the route of administration, the size (body weight, body surface) and/or condition (the age and general health) of the patient, and the general state of the patient's own immune system. The proper dose can be adjusted according to the judgment of the attending physician, in order to obtain the optimal therapeutic effect.

A therapeutically effective amount of an antibody construct of the invention preferably results in a decrease in severity of disease symptoms, an increase in frequency or duration of disease symptom-free periods or a prevention of impairment or disability due to the disease. In the treatment of CLDN18.2-expressing tumors, a therapeutically effective amount of the antibody construct of the invention preferably inhibits tumor cell growth by at least about 20%, at least about 40%, at least about 50%, at least about 60%, at least about 70%, at least about 80%, or at least about 90% relative to untreated patients. The ability of a compound to inhibit tumor growth may also be evaluated in an animal model predictive of efficacy in human tumors.

In a further embodiment, the invention provides a kit comprising an antibody construct of the invention, an antibody construct produced according to the process of the invention, a polynucleotide of the invention, a vector of the invention, and/or a host cell of the invention. In the context of the present invention, the term “kit” means two or more components—one of which corresponding to the antibody construct, the pharmaceutical composition, the polynucleotide, the vector or the host cell of the invention—packaged together in a container, recipient or otherwise. A kit can hence be described as a set of products and/or utensils that are sufficient to achieve a certain goal, which can be marketed as a single unit.

It is envisaged that a further component of the kit of the invention is an agent, preferably an antibody or antibody construct, which binds to a protein of the immune checkpoint pathway (such as PD-1 or CTLA-4) or to a co-stimulatory immune checkpoint receptor (such as 4-1BB). These agents are described in more detail herein above. According to one embodiment, the kit comprises an antibody construct of the invention and an antibody or antibody construct which binds to PD-1. Anti-PD-1 binding proteins useful for this purpose are e.g. described in detail in PCT/US2019/013205. In certain embodiment, the kit allows for the simultaneous and/or sequential administration of the components.

The kit may comprise one or more recipients (such as vials, ampoules, containers, syringes, bottles, bags) of any appropriate shape, size and material (preferably waterproof, e.g. plastic or glass) containing the antibody construct or the pharmaceutical composition of the present invention in an appropriate dosage for administration (see above). The kit may additionally contain directions for use (e.g. in the form of a leaflet or instruction manual), means for administering the antibody construct or the pharmaceutical composition of the present invention such as a syringe, pump, infuser or the like, means for reconstituting the antibody construct of the invention and/or means for diluting the antibody construct of the invention.

The invention also provides kits for a single-dose administration unit. The kit of the invention may also contain a first recipient comprising a dried/lyophilized antibody construct or pharmaceutical composition and a second recipient comprising an aqueous formulation. In certain embodiments of this invention, kits containing single-chambered and multi-chambered pre-filled syringes are provided.

The present invention refers to the following items:

Item 1. An antibody construct comprising a first domain which binds to CLDN18.2 (preferably having SEQ ID NO: 1) on the surface of a target cell and a second domain which binds to CD3 on the surface of a T cell.

Item 2. An antibody construct according to item 1, wherein the first domain binds to the first extracellular loop (loop 1) of CLDN18.2, the loop preferably having SEQ ID NO: 2.

Item 3. An antibody construct according to item 1 or 2 comprising
a first domain which binds to CLDN18.2 on the surface of a target cell, and
a second domain which binds to human CD3 on the surface of a T cell, wherein the first domain binds to the same epitope of CLDN18.2 as an antibody or antibody construct comprising a domain which binds to CLDN18.2 on the surface of a target cell and which comprises:
a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
c) a VH region as depicted in SEQ ID NO: 127, and a VL region as depicted in SEQ ID NO: 128; or
d) a VH region as depicted in SEQ ID NO: 139, and a VL region as depicted in SEQ ID NO: 140.

Item 4. An antibody construct according to any one of the preceding items, wherein the antibody construct competes for binding with an antibody or antibody construct comprising a domain which binds to CLDN18.2 on the surface of a target cell and which comprises:
a) a VH region comprising CDR-H1 as depicted in SEQ ID NO: 121, CDR-H2 as depicted in SEQ ID NO: 122, and CDR-H3 as depicted in SEQ ID NO: 123, and a VL region comprising CDR-L1 as depicted in SEQ ID NO: 124, CDR-L2 as depicted in SEQ ID NO: 125 and CDR-L3 as depicted in SEQ ID NO: 126;
b) a VH region comprising CDR-H1 as depicted in SEQ ID NO: 133, CDR-H2 as depicted in SEQ ID NO: 134, and CDR-H3 as depicted in SEQ ID NO: 135, and a VL region comprising CDR-L1 as depicted in SEQ ID NO: 136, CDR-L2 as depicted in SEQ ID NO: 137 and CDR-L3 as depicted in SEQ ID NO: 138;
c) a VH region as depicted in SEQ ID NO: 127, and a VL region as depicted in SEQ ID NO: 128; or
d) a VH region as depicted in SEQ ID NO: 139, and a VL region as depicted in SEQ ID NO: 140.

Item 5. An antibody construct according to any one of the preceding items, wherein the first domain of the antibody construct binds to a CLDN18.2 mutant on the surface of a target cell having the amino acid sequence as depicted in SEQ ID NO: 22, and optionally also binds to a CLDN18.2 mutant on the surface of a target cell having the amino acid sequence as depicted in SEQ ID NO: 24, but does not bind to a CLDN18.2 mutant on the surface of a target cell having the amino acid sequence as depicted in SEQ ID NO: 23;

Item 6. An antibody construct according to any one of the preceding items, wherein the first domain of the antibody construct binds to a CLDN18.2 mutant on the surface of a target cell having the amino acid sequence as depicted in SEQ ID NO: 14 and/or to a CLDN18.2 mutant on the surface of a target cell having the amino acid sequence SEQ ID NO 15, and optionally also binds to one or more CLDN18.2 mutants on the surface of a target cell having an amino acid sequence selected from the group consisting of those depicted in SEQ ID NOs: 11, 12, 13, 16, 17, 19, 20 and 21, but does not bind to a CLDN18.2 mutant on the surface of a target cell having the amino acid sequence as depicted in SEQ ID NO: 18;

Item 7. An antibody construct according to any one of the preceding items, wherein the first domain of the antibody construct binds to human CLDN18.2 on the surface of a target cell, wherein Glu (E) at position 56 of human CLDN18.2 is essential for the binding of the first domain, and Ala (A) at position 42 and/or Asn (N) at position 45 of human CLDN18.2 is/are not essential for the binding of the first domain; and/or

Item 8. An antibody construct according to any one of the preceding items, wherein the first domain of the antibody construct binds to an epitope of CLDN18.2 comprising the amino acid sequence as depicted in SEQ ID NO: 266, but not comprising the amino acid sequence as depicted in SEQ ID NO: 265, and optionally also not comprising the amino acid sequence as depicted in SEQ ID NO: 267.

Item 9. The antibody construct according to any one of the preceding items, wherein the second domain binds to human CD3 epsilon and to Callithrix jacchus or Saimiri sciureus CD3 epsilon.

Item 10. The antibody construct according to any one of the preceding items, wherein
a) the antibody construct, the first domain and/or the second domain are human or humanized,
b) the antibody construct is a single chain antibody construct,
c) the first domain is in the format of an scFv,
d) the second domain is in the format of an scFv,
e) the first and the second domain are connected via a linker, preferably a peptide linker, more preferably a glycine/serine linker, and/or
f) the antibody construct comprises a domain providing an extended serum half-life, such as an Fc-based domain.

Item 11. The antibody construct according to any one of the preceding items, wherein the first domain does not bind or does not significantly bind to CLDN18.1, CLDN1, CLDN2, CLDN3, CLDN4, CLDN6, and/or CLDN9.

Item 12 The antibody construct according to any one of the preceding items, wherein
a) the affinity of the first domain to CLDN18.2 (e.g. hu CLDN18.2) is ≤100 nM, ≤90 nM, ≤80 nM, ≤70 nM, ≤60 nM, ≤50 nM, ≤40 nM, ≤30 nM, or ≤20 nM, as preferably measured in a cell-based assay, such as a Scatchard assay;
b) the affinity of the second domain for CD3 (e.g. hu CD3, e.g. hu CD3 epsilon) is ≤100 nM, ≤90 nM, ≤80 nM, ≤70 nM, ≤60 nM, ≤50 nM, ≤40 nM, ≤30 nM, ≤20 nM, or ≤10 nM, as preferably measured in a surface plasmon resonance assay, such as a Biacore assay; and/or
c) the EC₅₀ value of the antibody construct is ≤500 pM, ≤400 pM, ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM, as preferably measured in a cytotoxicity assay with CLDN18.2 positive target cells (such as SNU-601, SNU-620 or CLDN18.2 transfected CHO cells) and stimulated enriched (human) CD8 positive T cells or unstimulated (human) peripheral blood mononuclear cells as effector cells.

Item 13. The antibody construct according to any one of the preceding items, wherein the first domain comprises a VH region comprising CDR-H1, CDR-H2 and CDR-H3 and a VL region comprising CDR-L1, CDR-L2 and CDR-L3 selected from the group consisting of:
a) CDR-H1 as depicted in SEQ ID NO: 121, CDR-H2 as depicted in SEQ ID NO: 122, and CDR-H3 as depicted in SEQ ID NO: 123, CDR-L1 as depicted in SEQ ID NO: 124, CDR-L2 as depicted in SEQ ID NO: 125 and CDR-L3 as depicted in SEQ ID NO: 126; and
b) CDR-H1 as depicted in SEQ ID NO: 133, CDR-H2 as depicted in SEQ ID NO: 134, and CDR-H3 as depicted in SEQ ID NO: 135, and CDR-L1 as depicted in SEQ ID NO: 136, CDR-L2 as depicted in SEQ ID NO: 137 and CDR-L3 as depicted in SEQ ID NO: 138.

Item 14. The antibody construct according to any one of the preceding items, wherein the first domain comprises a VH region having an amino acid sequence as depicted in SEQ ID NO: 127 or SEQ ID NO: 139.

Item 15. The antibody construct according to one of the preceding items, wherein the first domain comprises a VL region having an amino acid sequence as depicted in SEQ ID NO: 128 or SEQ ID NO: 140.

Item 16. The antibody construct according to one of the preceding items, wherein the first domain comprises a VH region and a VL region having an amino acid sequence as depicted in SEQ ID NOs: 127+128 or SEQ ID NOs: 139+140.

Item 17. The antibody construct according to one of the preceding items, wherein the first domain comprises a polypeptide having an amino acid sequence as depicted in SEQ ID NO: 129 or SEQ ID NO: 141.

Item 18. The antibody construct according to one of the preceding items, comprising or consisting of a polypeptide having an amino acid sequence selected from the group of those depicted in SEQ ID NO: 131, SEQ ID NO: 132, SEQ ID NO: 143, and SEQ ID NO: 144.

Item 19. A polynucleotide encoding an antibody construct as defined in any one of the preceding items.

Item 20. A vector comprising a polynucleotide as defined in item 19.

Item 21. A host cell transformed or transfected with the polynucleotide as defined in item 19 or with the vector as defined in item 20.

Item 22. A process for producing an antibody construct as defined in any one of items 1 to 18, said process comprising culturing a host cell as defined in item 21 under conditions allowing the expression of said antibody construct and recovering the produced antibody construct from the culture.

Item 23. A pharmaceutical composition comprising an antibody construct as defined in any one of items 1 to 18, or produced according to the process of item 22.

Item 24. The antibody construct according to any one of items 1 to 18, or produced according to the process of item 22, for use in the prevention, treatment or amelioration of a disease, preferably a neoplasm, more preferably a CLDN18.2 positive neoplasm.

Item 25. The antibody construct according to item 24, wherein the disease or neoplasm is selected from the group consisting of gastrointestinal cancer, ovarian cancer and lung cancer.

Item 26. The antibody construct according to item 25, wherein the gastrointestinal cancer is selected from the group consisting of gastric cancer, esophageal cancer, gastroesophageal cancer, pancreatic cancer, and colorectal cancer.

Item 27. A kit comprising an antibody construct as defined in any one of items 1 to 18, an antibody construct produced according to the process of item 22, a polynucleotide as defined in item 19, a vector as defined in item 20, and/or a host cell as defined in item 21.

Item 28. The pharmaceutical composition of item 23 or the kit of item 27, further comprising an agent, preferably an antibody or antibody construct, which binds to a protein of the immune checkpoint pathway (such as PD-1 or CTLA-4) or to a co-stimulatory immune checkpoint receptor (such as 4-1BB).

Item 29. The pharmaceutical composition of item 23 or 28, or the kit of item 27 or 28, for therapeutic use, preferably for use in the prevention, treatment or amelioration of a disease, preferably a neoplasm, more preferably a CLDN18.2 positive neoplasm.

The Figures show:

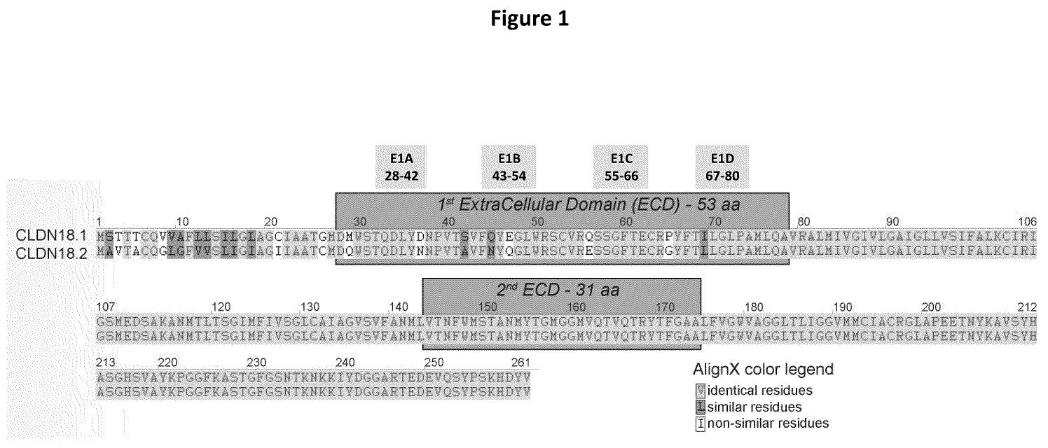

FIG. 1

Alignment of the human CLDN18.1 and CLDN18.2 amino acid sequence. The first and the second extracellular domains (=extracellular loops) are highlighted, as well as the amino acid positions differing between CLDN18.1 and CLDN18.2. Within the extracellular loop 1, CLDN18.2 and CLDN18.1 differ in eight positions. See also Example 1.

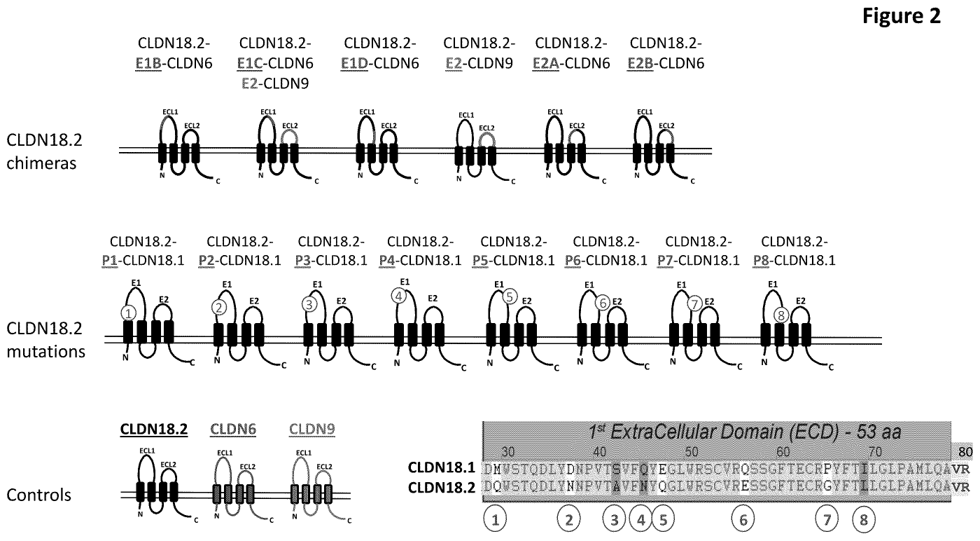

FIG. 2

The figure depicts the CLDN18.2 constructs (chimeras/point mutations) that were generated for the epitope mapping analysis of Example 2. It also shows the sequence alignment between human CLDN18.1 loop 1 and human CLDN18.2 loop 1 and highlights the eight positions P1-P8 in which these two molecules differ. See also Example 1.

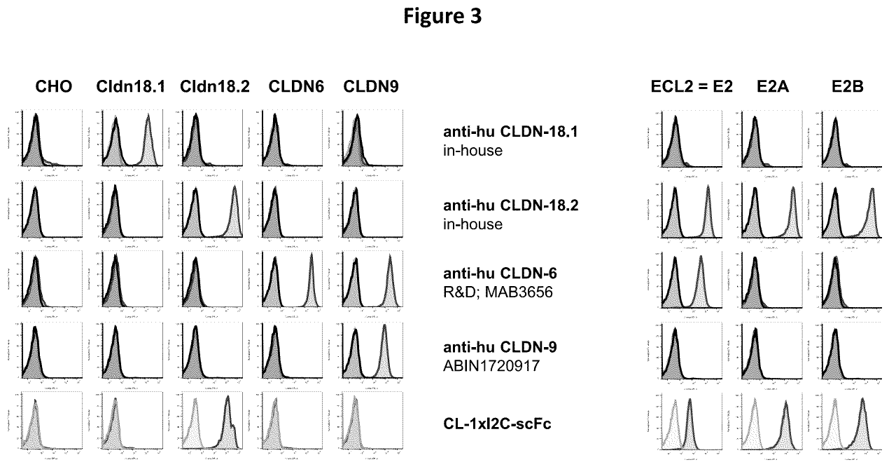

FIG. 3

Results of epitope mapping analysis, see Example 2. The figure shows FACS analyses of untransfected CHO cells, as well as of CHO cells transfected with hu CLDN18.1, hu CLDN18.2, hu CLDN6 and hu CLDN9 (left-hand side). On the right hand side, the figure shows FACS analyses of CHO cells transfected with three different chimeric hu CLDN18.2 constructs: The amino acid sequence of the entire loop 2 (ECL2, E2) was exchanged for a sequence originating from human CLDN9, and the regions E2A and E2B were exchanged for a counterpart sequence of human CLDN6. All antibodies/antibody constructs were tested at a concentration of 5 μg/ml.

FIG. 4

Results of epitope mapping analysis, see Example 2. The figure shows FACS analyses of untransfected CHO cells, as well as of CHO cells transfected with hu CLDN18.1, hu CLDN18.2, hu CLDN6 and hu CLDN9 Furthermore, the figure shows FACS analyses of CHO cells transfected with eleven different hu CLDN18.2 constructs having one, two or three point mutations at the indicated positions P1-P8 (for further details, see also Example 1), as well as of CHO cells transfected with four different chimeric hu CLDN18.2 constructs: The amino acid sequence of the entire loop 2 (ECL2, E2) was exchanged for a sequence originating from human CLDN9, and the regions E1B, E1D and E1C were exchanged for a counterpart sequence of human CLDN6 (for further details, see also Example 1). All monospecific antibodies were tested at a concentration of 5 μg/ml, while the CD3×CLDN18.2 bispecific antibody constructs were tested at a concentration of 20 μg/ml.
Row 1: in-house α-hu CLDN-18.1
Row 2: in-house α-hu CLDN-18.2
Row 3: α-hu CLDN-6 (R&D; MAB3656)
Row 4: α-hu CLDN-9 (ABIN1720917)
Row 5: CL-1×I2C-scFc
Row 6: CL-2×I2C-scFc
Row 7: CL-4×I2C-scFc
Row 8: CL-3×I2C-scFc

FIG. 5

Results of the FACS assay described in Example 5.

FIG. 6

Results of the FACS assay described in Example 6.

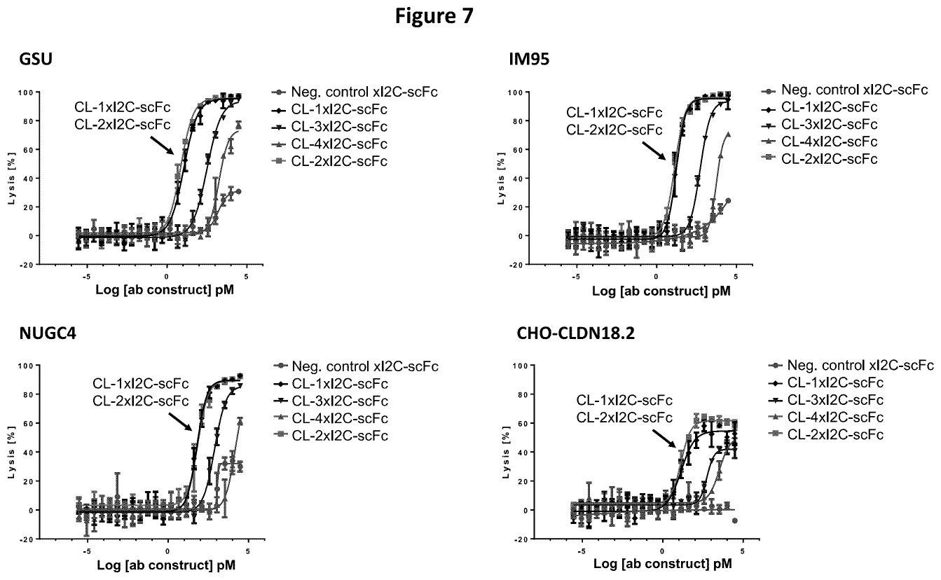

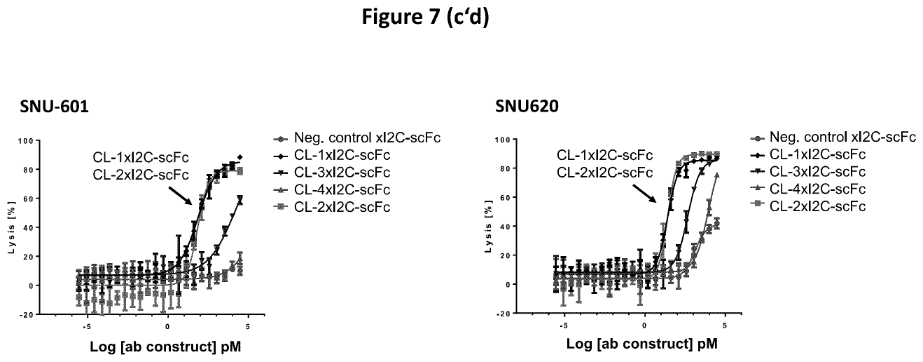

FIG. 7

Results of the FACS-based cytotoxicity assays described in Example 7.4. In all six cell lines tested (five natural expresser cell lines and CHO cells transfected with hu CLDN18.2), the antibody constructs of the invention (CL-1 and CL-2) showed significantly higher EC50 values compared with the control constructs binding to a different CLDN18.2 epitope (CL-3 and CL-4).

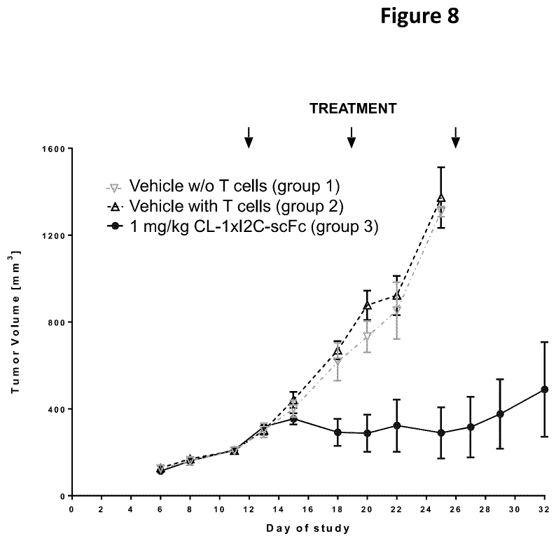

FIG. 8

Anti-tumor activity of a CLDN18.2×CD3 antibody construct (mean tumor volume with standard error of mean (SEM)). See Example 13.

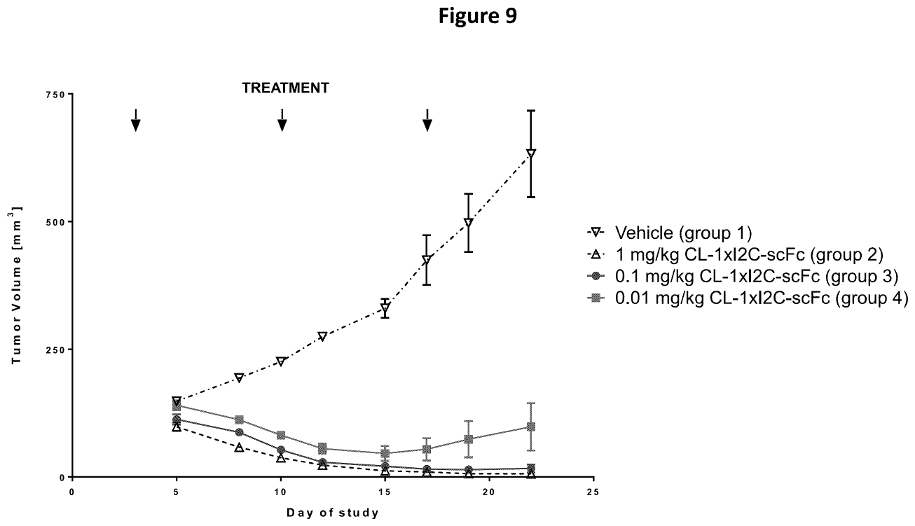

FIG. 9

Anti-tumor formation activity of a CLDN18.2×CD3 antibody construct (mean tumor volume with standard error of mean (SEM)). See Example 14.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

It should be understood that the above description and the below examples provide exemplary arrangements, but the present invention is not limited to the particular methodologies, techniques, protocols, material, reagents, substances, etc., described herein and as such can vary. The terminology used herein is for the purpose of describing particular embodiments only, and is not intended to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### Example 1

### Generation of CHO Cells Expressing CLDN18.2 Mutations

For the purposes of epitope mapping, E1 (extracellular loop 1; ECL1, SEQ ID NO: 2) of CLDN18.2 was divided into four sub-domains (E1A, E1B, E1C and E1D), and E2 (extracellular loop 2; ECL2, SEQ ID NO: 3) was divided into two sub-domains (E2A and E2B). The amino acid sequence of the respective epitope region (loop/domain or sub-domain) of human CLDN18.2 (E1, E1A, E1B, E1C, E1D, E2, E2A and E2B) was exchanged for a counterpart sequence of human CLDN6, with two exceptions (see FIGS. 1 and 2): E2 was exchanged for a sequence originating from human CLDN9; and in the construct having a mutated E1C (exchange of CLDN18.2 E1C for the counterpart CLDN6 region), E2 of CLDN18.2 was also exchanged for a sequence originating from human CLDN9. The latter served as a means to detect expression of the CLDN18.2/CLDN6-chimera. The expression of all chimeric constructs in CHO cells was verified via FACS analysis (see below). As there was no proof of expression for the E1 and the E1A chimeric constructs, these constructs were not used for the epitope mapping analysis. The following constructs were hence generated and used for epitope mapping:
CLDN18.2-E1B (CLDN6)→SEQ ID NO: 22
CLDN18.2-E1C (CLDN6)-E2 (CLDN9)→SEQ ID NO: 23
CLDN18.2-E1D (CLDN6)→SEQ ID NO: 24
CLDN18.2-E2 (CLDN9)→SEQ ID NO: 25
CLDN18.2-E2A (CLDN6)→SEQ ID NO: 26
CLDN18.2-E2B (CLDN6) →SEQ ID NO: 27

The amino acid sequences of human CLDN18.2-ECL2 and human CLDN18.1-ECL2 are identical, but within extracellular loop 1, CLDN18.2 and CLDN18.1 differ in eight positions (29, 37, 42, 45, 47, 56, 65 and 69). Therefore, additional CLDN18.2 mutants were generated and expressed in CHO cells, in which these eight CLDN18.2 positions “P1” to “P8” were exchanged by their respective CLDN18.1 counterparts, either individually or in a group of two or three positions. The following constructs were hence generated:
CLDN18.2-P1-CLDN18.1 (Q29M mutation)→SEQ ID NO: 11
CLDN18.2-P2-CLDN18.1 (N37D mutation)→SEQ ID NO: 12
CLDN18.2-P1/P2-CLDN18.1 (Q29M/N37D mutation)→SEQ ID NO: 13
CLDN18.2-P3-CLDN18.1 (A42S mutation)→SEQ ID NO: 14
CLDN18.2-P4-CLDN18.1 (N45Q mutation)→SEQ ID NO: 15
CLDN18.2-P5-CLDN18.1 (Q47E mutation)→SEQ ID NO: 16
CLDN18.2-P3/P4/P5-CLDN18.1 (A42S/N45Q/Q47E mutation)→SEQ ID NO: 17
CLDN18.2-P6-CLDN18.1 (E56Q mutation)→SEQ ID NO: 18
CLDN18.2-P7-CLDN18.1 (G65P mutation)→SEQ ID NO: 19
CLDN18.2-P8-CLDN18.1 (L69I mutation)→SEQ ID NO: 20
CLDN18.2-P7/P8-CLDN18.1 (G65P/L69I mutation)→SEQ ID NO: 21

For the generation of CHO cells expressing the above constructs, as well as of CHO cells expressing hu-CLDN18.2, hu-CLDN18.1, hu-CLDN6 and hu-CLDN9 (SEQ ID NOs: 1, 4, 9 and 10) as controls, the respective coding sequences were cloned into a plasmid designated pEF-DHFR (pEF-DHFR is described in Raum et al. Cancer Immunol Immunother 50 (2001) 141-150). All cloning procedures were carried out according to standard protocols (Sambrook, Molecular Cloning; A Laboratory Manual, 3rd edition, Cold Spring Harbour Laboratory Press, Cold Spring Harbour, New York (2001)). For each construct, a corresponding plasmid was transfected into DHFR deficient CHO cells for eukaryotic expression, as described by Kaufman R. J. (1990) Methods Enzymol. 185, 537-566.

The expression of the above constructs on CHO cells was verified in a FACS assay using antibodies against CLDN18.2 (in-house monoclonal anti-hu CLDN18.2 antibody), CLDN18.1 (in-house monoclonal anti-hu CLDN18.1 antibody), CLDN6 (R&D mouse anti-human CLDN6 monoclonal antibody MAB3656) and CLDN9 (rat anti-human CLDN9 monoclonal antibody ABIN1720917), respectively, at a concentration of 5 μg/ml. As negative control, cells were incubated with an isotype control antibody (BD 553454/R&D MAB0041/R&D MAB0061) instead of the first antibody. Bound monoclonal antibody was detected with a secondary anti-mouse/anti-rat/anti-human IgG Fc-gamma-PE (Jackson ImmunoResearch 115-116-071/112-116-071/109-116-098). The samples were measured by flow cytometry.

### Example 2

### Epitope Mapping of Anti-CLDN18.2 Antibody Constructs

CHO cells transfected with the constructs described in Example 1 were stained with purified CLDN18.2×CD3 antibody constructs at a concentration of 20 μg/ml. Bound antibody constructs were detected with an anti-human IgG Fc-gamma-PE (Jackson ImmunoResearch; 1:100). All antibodies were diluted in PBS/2% FCS. As negative control, cells were incubated with PBS/2% FCS followed by the anti-human IgG Fc-gamma-PE. The samples were measured by flow cytometry.

The results of the epitope mapping analysis are shown in FIGS. 3 and 4. When a loss of the FACS signal is observed for cells expressing a certain CLDN18.2 chimera or mutation, the respective CLDN18.2×CD3 antibody construct is assumed to bind to the epitope (loop/domain/sub-domain) or to the specific amino acid that was exchanged in this CLDN18.2 chimeric or mutated molecule. In other words, this epitope region or amino acid is required for the binding of the CLDN18.2×CD3 antibody construct that was analyzed. In addition to the control antibodies which were used to demonstrate proper expression of the respective target, the following CLDN18.2×CD3 antibody constructs were specifically tested in the epitope mapping analysis:
CL-1×I2C-scFc (SEQ ID NO: 132)
CL-2×I2C-scFc (SEQ ID NO: 144)
CL-3×I2C-scFc (SEQ ID NO: 149)
CL-4×I2C-scFc (SEQ ID NO: 154)

While CL-1×12C-scFc and CL-2×I2C-scFc are antibody constructs according to the invention, CL-3×I2C-scFc and CL-4×I2C-scFc have anti-CLDN18.2 VH and VL regions that are disclosed as SEQ ID NOs: 8+15 and SEQ ID NOs: 6+11, respectively, of WO 2014/075788.

As shown in FIG. 4, the CL-3×I2C-scFc antibody construct clearly requires positions P3 (A42), P4 (N45) and P6 (E56) for its specific binding to CLDN18.2. Position P4 is located within the sub-domain denominated E1B, and position P6 is located within the sub-domain denominated E1C. Consequently and likewise, an exchange of these sub-domains with the CLDN6 counterpart sequence leads to a loss of the FACS signal. The observation that these three positions are relevant for the binding of CL-3×I2C-scFc to CLDN18.2 confirms previously published results. The antibody construct denominated CL-4×12C-scFc has a very similar binding pattern (see FIG. 4).

In contrast, both antibody constructs CL-1×I2C-scFc and CL-2×I2C-scFc clearly require position P6 (E56) for their specific binding to CLDN18.2. However, the exchange of other positions, in particular of P3 and P4, does not appear to have any impact on the binding of CL-1×I2C-scFc or CL-2×12C-scFc to CLDN18.2. In line with this observation, the exchange of sub-domain E1C (in which position P6 is located) with the CLDN6 counterpart sequence—but not the exchange of E1B or E1D—leads to a loss of the FACS signal. The epitope mapping result depicted in FIG. 4 hence shows that CL-1×I2C-scFc and CL-2×I2C-scFc bind to the same epitope within ECL1 of CLDN18.2, and this epitope differs from the one of CL-3×I2C-scFc and CL-4×I2C-scFc.

### Example 3

### Biacore-Based Determination of Affinity to Human and Cynomolgus CD3 and FcRn

Biacore analysis experiments were performed using recombinant human/macaque CD3-ECD (ECD=extracellular domain) fusion proteins with chicken albumin to determine target binding of the antibody constructs of the invention.

In detail, CM5 Sensor Chips (GE Healthcare) were immobilized with approximately 600-800 RU of the respective recombinant antigen using acetate buffer pH 4.5 according to the manufacturer's manual. The CLDN18.2×CD3 antibody construct was loaded in a dilution series of the following concentrations: 50 nM, 25 nM, 12.5 nM, 6.25 nM and 3.13 nM diluted in HBS-EP running buffer (GE Healthcare). Flow rate was 30 μl/min for 3 min, then HBS-EP running buffer was applied for 8 min to 20 min again at a flow rate of 30 μl/ml. Regeneration of the chip was performed using 10 mM glycine 10 mM NaCl pH 1.5 solution. Data sets were analyzed using BiaEval Software. In general, two independent experiments were performed.

The CLDN18.2×CD3 antibody constructs according to the invention showed high affinities to human CD3 in the nanomolar range. Binding to macaque CD3 was balanced, also showing affinities in similar ranges. The affinity values as well as the calculated affinity gap are shown in Table 2.

TABLE 2
Affinities of CLDN18.2 × CD3 antibody constructs
to human and macaque CD3 as determined by Biacore analysis,
as well as the calculated interspecies affinity gaps.
The constructs in rows 3-5 were measured in a different
(separate) assay than the constructs in rows 1 and 2.

| | CLDN18.2 × CD3 | KD hu CD3 | KD cyno CD3 | Affinity gap |
|---|---|---|---|---|
| | antibody construct | [nM] | [nM] | KD mac/KD hu |
| 1 | CL-1 × I2C-scFc | 23.5 ± 1.0 | 19.7 ± 1.1 | 0.84 |
| 2 | CL-1 × I2C-6His | 4.0 ± 0.2 | 3.4 ± 0.2 | 0.85 |
| 3 | CL-2 × I2C-scFc | 8.32 ± 0.64 | 4.67 ± 0.18 | 0.56 |
| 4 | CL-3 × I2C-scFc | 14.50 ± 0.07 | 7.91 ± 0.37 | 0.55 |
| 5 | CL-4 × I2C-scFc | 15.30 ± 2.55 | 9.19 ± 0.16 | 0.60 |

Likewise, a balanced binding to human and cyno FcRn was confirmed via Biacore assays for the constructs denominated CL-1×12C-scFc, CL-2×12C-scFc, CL-3×12C-scFc, and CL-4×12C-scFc.

### Example 4

### Scatchard-Based Analysis of the Affinity to Human and Macaque CLDN18.2 on Target Antigen Positive Cells

The affinities of CLDN18.2×CD3 antibody constructs to CHO cells transfected with human or macaque CLDN18.2 were determined by Scatchard analysis. For this analysis, saturation binding experiments were performed using a monovalent detection system to precisely determine monovalent binding of the CLDN18.2×CD3 antibody constructs to the respective cell line.

2×10⁴ cells of the CHO cell line recombinantly expressing human CLDN18.2 were incubated each with 50 μl of a dilution series (twelve dilutions at 1:2) of the respective antibody construct (until saturation was reached) starting at 100-200 nM followed by 16 h incubation at 4° C. under agitation and one residual washing step. Then, the cells were incubated for another hour with 30 μl of an Alexa Fluor™ 488-conjugated AffiniPure™ Fab fragment goat anti-human IgG (H+L) solution. After one washing step, the cells were resuspended in 150 μl FACS buffer containing 3.5% formaldehyde, incubated for further 15 min, centrifuged, resuspended in FACS buffer and analyzed via FACS software. Data were generated from two independent sets of experiments, each using triplicates. Respective one site-specific binding evaluation was calculated to extrapolate maximal binding (Bmax). The concentrations of the antibody constructs at half-maximal binding were determined reflecting the respective KDs. Values of triplicate measurements were plotted as hyperbolic curves and as S-shaped curves to demonstrate proper concentration ranges from minimal to optimal binding.

Values depicted in Table 3 were derived from two independent experiments per antibody construct. Cell based Scatchard analysis confirmed that the CLDN18.2×CD3 antibody constructs of the invention are nanomolar in affinity to human CLDN18.2 and—due to sequence identity of human and macaque extracellular domains—present with a cyno/human interspecies affinity gap of 1.

TABLE 3
Affinities (KD) of CLDN18.2 × CD3 antibody constructs to
CLDN18.2 as determined in cell based Scatchard analysis. Antibody
constructs were measured in two independent experiments, each
using a dilution series.

| CLDN18.2 × CD3 | Cell based affinity | Affinity gap |
|---|---|---|
| antibody construct | hu CLDN18.2 [nM] | KD mac/KD hu* |
| CL-1 × I2C-scFc | 56.8 ± 10.7 | 1 |
| CL-1 × I2C-6His | 13.9 ± 1.8 | 1 |

*Human and cynomolgus CLDN18.2 share the identical amino acid sequence in the extracellular domains. Therefore the affinity gap equals “1” per definition

In a separate Scatchard assay carried out under the same conditions, a cell-based affinity for human CLDN18.2 of 11.13+2.72 was measured for CL-2×I2C-scFc under the same conditions.

### Example 5

### Confirmation of Binding to CLDN18.2 and Human/Cyno CD3 Expressing Cells

For confirmation of binding to human CLDN18.2 and CD3 and to cyno CD3, antibody constructs of the invention were tested by flow cytometry using
CHO cells transfected with human CLDN18.2,
CD3-expressing human T cell leukemia cell line HPB-all (DSMZ, Braunschweig, ACC483), and
the cynomolgus CD3-expressing T cell line LnPx 4119

For flow cytometry 200,000 cells of the respective cell lines were incubated for 60 min at 4° C. with the purified antibody construct at a concentration of 5 μg/ml. After washing, bound antibody constructs having an Fc domain were detected with a goat anti-human Fc-gamma-PE (1:100) for 30 min at 4° C. The antibody construct having a his-tag was detected with an in-house mouse antibody specific for the CD3 binding part, followed by a goat anti-mouse Fc-gamme-PE (1:100) for 30 min at 4° C. Samples were measured by flow cytometry. Non-transfected CHO cells were used as negative control.

The results are shown in FIG. 5. The CLDN18.2×CD3 antibody constructs of the invention stained CHO cells transfected with human CLDN18.2. Human and cyno T cell lines expressing CD3 were also recognized by the antibody constructs. There was no staining of non-transfected CHO cells, and no staining of the CLDN18.2-transfected CHO cells by the negative control anti-EGFRVIII×I2C-scFc antibody construct.

### Example 6

### Confirmation of the Absence of Binding to Human CLDN18.2 Paralogues

Human CLDN18.2 paralogues CLDN18.1, CLDN1, CLDN2, CLDN3, CLDN4, CLDN6 and CLDN9 were stably transfected into CHO cells. The sequences of the paralogues as used in the present example are depicted in SEQ ID Nos: 4-10. Protein expression was confirmed in FACS analyses with antibodies specific for the respective paralogues:
CLDN1: rat-anti-human (5 μg/ml final) stock: 100 μg/ml R&D, MAB4618
CLDN2: in-house mouse Ab (1:100-200)
CLDN3: mouse-anti-human (5 μg/ml final) stock: 500 μg/ml R&D, MAB4620
CLDN4: mouse-anti-human (5 μg/ml final) stock: 500 μg/ml R&D, MAB4219
CLDN6: mouse-anti human (5 μg/ml final) stock: 500 μg/ml R&D, MAB3656
CLDN9: rat-anti-human (5 μg/ml final) stock: 2 mg/ml, antibodies-online.com, ABIN1720917
CLDN18.1: in-house monoclonal mouse Ab (5 μg/ml final)

The transfected CHO cells were incubated for 60 min at 4° C. with the respective antibody (see above) at a concentration of 5 μg/ml, followed by the respective PE conjugated antibody goat-anti-mouse IgG, Fc-gamma fragment PE conjugated (1:100), Jackson 115-116-071 or goat-anti-rat IgG, Fc-gamma fragment PE conjugated (1:100) Jackson, 112-116-071 for 30 min at 4° C.

The flow cytometry assay was carried out as described in Example 5. The results are shown in FIG. 6. The analysis confirmed that the CLDN18.2×CD3 antibody construct of the invention that was tested in this assay does not cross-react with the human CLDN18.2 paralogues.

### Example 7

### Cytotoxic Activity

The potency of CLDN18.2×CD3 antibody constructs of the invention in redirecting effector T cells against CLDN18.2-expressing target cells was analyzed in different in vitro cytotoxicity assays:
The potency of CLDN18.2×CD3 antibody constructs in redirecting stimulated human CD8+ effector T cells against human CLDN18.2-transfected CHO cells was measured in a 48 hour FACS-based cytotoxicity assay.
The potency of CLDN18.2×CD3 antibody constructs in redirecting a macaque T cell line against human CLDN18.2-transfected CHO cells was measured in a 48 hour FACS-based cytotoxicity assay.
The potency of CLDN18.2×CD3 antibody constructs in redirecting the T cells in unstimulated human PBMC against human CLDN18.2-transfected CHO cells (along with a negative control using human CLDN18.1-transfected CHO cells) was measured in a 48 hour FACS-based cytotoxicity assay.
The potency of CLDN18.2×CD3 antibody constructs in redirecting the T cells in unstimulated human PBMC against a CLDN18.2 positive human gastric cancer line such as SNU-601 or SNU-620 was measured in a 48 hour FACS-based cytotoxicity assay.

### Example 7.1

### Chromium Release Assay with Stimulated Human T Cells

Stimulated T cells enriched for CD8⁺ T cells are obtained as follows: A petri dish (145 mm diameter) is coated with a commercially available anti-CD3 specific antibody (OKT3, Orthoclone) in a final concentration of 1 μg/ml for 1 hour at 37° C. Unbound protein is removed by one washing step with PBS. 3-5×10⁷ human PBMC are added to the precoated petri dish in 120 ml of RPMI 1640 with stabilized glutamine/10% FCS/20 U/ml IL-2 and stimulated for 2 days. On the third day, the cells are collected and washed once with RPMI 1640. IL-2 is added to a final concentration of 20 U/ml, and the cells are cultured again for one day in the same cell culture medium as above. CD8⁺ cytotoxic T lymphocytes (CTLs) are enriched by depletion of CD4⁺ T cells and CD56⁺ NK cells using Dynal-Beads according to the manufacturer's protocol.

Human CLDN18.2-transfected CHO target cells are washed twice with PBS and labeled with 11.1 MBq ⁵¹Cr in a final volume of 100 μl RPMI with 50% FCS for 60 minutes at 37° C. Subsequently, the labeled target cells are washed 3 times with 5 ml RPMI and then used in the cytotoxicity assay. The assay is performed in a 96-well plate in a total volume of 200 μl supplemented RPMI with an E:T ratio of 10:1. A starting concentration of 0.01-1 μg/ml of purified antibody construct and threefold dilutions thereof are used. Incubation time for the assay is 18 hours. Cytotoxicity is determined as relative values of released chromium in the supernatant relative to the difference of maximum lysis (addition of Triton-X) and spontaneous lysis (without effector cells). All measurements are carried out in quadruplicates. Measurement of chromium activity in the supernatants is performed in a gamma counter. Analysis of the results is carried out with appropriate software. EC50 values calculated by the analysis program from the sigmoidal dose response curves are used for comparison of cytotoxic activity.

### Example 7.2

### FACS-Based Cytotoxicity Assay with Unstimulated Human PBMC

### Isolation of Effector Cells

Human peripheral blood mononuclear cells (PBMC) were prepared by Ficoll density gradient centrifugation from enriched lymphocyte preparations (buffy coats). PBMC were prepared on the day of blood collection. After Ficoll density centrifugation and extensive washes with Dulbecco's PBS, remaining erythrocytes were removed from PBMC via incubation with erythrocyte lysis buffer (155 mM NH₄Cl, 10 mM KHCO₃, 100 UM EDTA). Platelets were removed via the supernatant upon centrifugation of PBMC at 100×g. Remaining lymphocytes mainly encompass B lymphocytes, T lymphocytes, NK cells and monocytes. PBMC were kept in culture at 37° C./5% CO2 in RPMI medium with 10% FCS.

Depletion of CD14⁺ and CD56⁺ cells

For depletion of CD14+ cells, human CD14 MicroBeads (Miltenyi Biotec, MACS, #130-050-201) were used. For depletion of NK cells, human CD56 MicroBeads (Miltenyi Biotec, MACS, #130-050-401) were used. PBMC were counted and centrifuged for 10 min at room temperature with 300×g. The supernatant was discarded and the cell pellet resuspended in MACS isolation buffer (80 μl/10⁷ cells; PBS, 0.5% (v/v) FBS, 2 mM EDTA). CD14 MicroBeads and CD56 MicroBeads (20 μl/10⁷ cells) were added and incubated for 15 min at 4-8° C. The cells were washed with MACS isolation buffer (1-2 ml/10⁷ cells). After centrifugation (see above), supernatant was discarded and cells resuspended in MACS isolation buffer (500 μl/10⁸ cells). CD14/CD56 negative cells were then isolated using LS Columns (Miltenyi Biotec, #130-042-401). PBMC w/o CD14+/CD56+ cells were cultured in RPMI complete medium (i.e. RPMI1640 supplemented with 10% FBS, 1× non-essential amino acids, 10 mM Hepes buffer, 1 mM sodium pyruvate and 100 U/ml penicillin/streptomycin) at 37° ° C. in an incubator until needed.

### Target Cell Labeling

For the analysis of cell lysis in flow cytometry assays, the fluorescent membrane dye DiOC₁₈ (DiO) (Molecular Probes) was used to label target cells (such as human CLDN18.2-transfected CHO cells) and distinguish them from effector cells. Briefly, cells were harvested, washed once with PBS and adjusted to 10⁶ cells/ml in PBS containing 2% (v/v) FBS and the membrane dye DiO (5 μl/10⁶ cells). After incubation for 3 min at 37° C., cells were washed twice in complete RPMI medium, and the cell number was adjusted to 1.25×10⁵ cells/ml. The vitality of cells was determined using 0.5% (v/v) isotonic EosinG solution.

### Flow Cytometry Based Analysis

This assay was designed to quantify the lysis of target cells (such as human CLDN18.2-transfected CHO cells) in the presence of serial dilutions of CLDN18.2×CD3 antibody constructs. Equal volumes of DiO-labeled target cells and effector cells (i.e., PBMC w/o CD14+ cells) were mixed, resulting in an E:T cell ratio of 10:1. 160 μl of this suspension were transferred to each well of a 96-well plate. 40 μl of serial dilutions of the CLDN18.2×CD3 antibody constructs to be analyzed (and possibly a negative control antibody construct such as an anti-CD3 (12C)-based bispecific antibody construct recognizing an irrelevant target antigen) or RPMI complete medium as an additional negative control were added. The antibody construct-mediated cytotoxic reaction proceeded for 48 hours in a 7% CO2 humidified incubator. Then cells were transferred to a new 96-well plate and loss of target cell membrane integrity was monitored by adding propidium iodide (PI) at a final concentration of 1 μg/ml. PI is a membrane impermeable dye that normally is excluded from viable cells, whereas dead cells take it up and become identifiable by fluorescent emission.

Samples were measured by flow cytometry on a FACSCanto II instrument and analyzed by FACSDiva software (both from Becton Dickinson). Target cells were identified as DiO-positive cells. PI-negative target cells were classified as living target cells. Percentage of cytotoxicity was calculated according to the following formula:

$$Cytotoxicity [ \% ] = \frac{{n}_{dead target cells}}{{n}_{target cells}} \times 100$$

$$n = number of events$$

Using the Prism software (GraphPad Software Inc.), the percentage of cytotoxicity was plotted against the corresponding antibody construct concentrations. Dose response curves were analyzed with the four parametric logistic regression models for evaluation of sigmoid dose response curves with fixed hill slope and EC50 values were calculated.

### Example 7.3

### Potency of Redirecting Unstimulated Human PBMC Against Target Cells

The cytotoxic activity of CLDN18.2×CD3 antibody constructs was analyzed in a FACS-based 48 h cytotoxicity assay using unstimulated human PBMC (CD14 neg./CD56 neg.) as effector cells and using as target cells in an E:T ratio of 10:1:
(1) CHO cells transfected with human CLDN18.2,
(2) CHO cells transfected with human CLDN18.1, and
(3) natural expresser cell lines SNU-601 and SNU-620.

The assay was carried out as described in Example 7.2 above. The results of the cytotoxicity assays are shown in Table 4.

TABLE 4
EC50 values [pM] of CLDN18.2 × CD3 antibody constructs
as measured in a 48-hour FACS-based cytotoxicity assay with
unstimulated human PBMC as effector cells.

| | CLDN18.2 × CD3 | |
|---|---|---|
| Target cells | antibody construct | EC50 [pM] |
| (1) | CL-1 × I2C-scFc | 5.2 |
| (1) | CL-1 × I2C-6His | 0.5 |
| (2) | CL-1 × I2C-scFc | 99396 |
| (2) | CL-1 × I2C-6His | 1985 |
| (3) | CL-1 × I2C-scFc | 104 |
| (3) | CL-1 × I2C-6His | 2.3 |
| (4) | CL-1 × I2C-scFc | 185 |
| (4) | CL-1 × I2C-6His | 12 |

Target cells: (1) CHO cells transfected with hu CLDN18.2; (2) CHO cells transfected with hu CLDN18.1; (3) SNU-601; (4) SNU-620.

The assay demonstrates that the antibody constructs of the invention do not exhibit significant undesired cytotoxic activity against CHO cells transfected with the CLDN18.2 paralogue CLDN18.1 (rows highlighted in grey). In the case of the construct denominated “CL-1×I2C-6His”, the factor between the EC₅₀ value for CLDN18.2-CHO and the EC50 value for CLDN18.1-CHO is almost 4.000. Moreover, in the case of the construct denominated “CL-1×I2C-scFc”, the factor between the EC₅₀ value for CLDN18.2-CHO and the EC50 value for CLDN18.1-CHO is almost 20.000.

Usually EC₅₀ values are expected to be lower when using target cells that express higher levels of CLDN18.2 on the cell surface compared with target cells having a lower target expression rate. Therefore, it is usually observed—and demonstrated in the present assay—that the use of CHO cells transfected with CLDN18.2 has a tendency to result in lower EC₅₀ values compared with the use of natural expressers.

### Example 7.4

### Potency of Redirecting Human PBMC Against Natural Expresser Target Cells

The cytotoxic activity of CLDN18.2×CD3 antibody constructs of the invention was compared against other CLDN18.2×CD3 antibody constructs which bind to a different epitope within the CLDN18.2 target (CL-3 and CL-4, see Example 2). Cancer cell lines stably expressing endogenous levels of CLDN18.2 (GSU, NUGC, IM95, SNU620, SNU601) and the CLDN18.2 negative control cell line AGS were stably labeled with luciferase (Luc). Furthermore, CHO cells were stably transfected to overexpress CLDN18.2. Two human T cell donors were used as a source for effector cells. The E:T ratio of the cytotoxicity assay was 10:1. The 1:3 serial titration of the antibody constructs started at a concentration of 30 nM. The assays were incubated for 48h at 37° C. Cytotoxicity readout was performed by the luciferase assay system ONE-Glo™ (Promega) for Luc labeled cells and the luminescent cell viability assay CellTiter-Glo™ (Promega) for CLDN18.2 transfected CHO cells.

With the exception of the sequences for the anti-CLDN18.2 VH and VL regions, the antibody constructs CL-3 and CL-4 were identical to the antibody constructs of the invention analyzed in the cytotoxicity assays of the present Example. The VH and VL regions of the two antibody constructs CL-3 and CL-4 are disclosed as SEQ ID NOs: 8+15 and SEQ ID NOs: 6+11, respectively, of WO 2014/075788. See also SEQ ID NOs: 145+146 and SEQ ID NOs: 150+151 as disclosed herein.

Results are shown in Table 5 below and in FIG. 7. Irrespective of the target cells, the antibody constructs of the present invention (here: CL-1, CL-2) were shown to have a significantly higher cytotoxic potency than the antibody constructs binding to a different CLDN18.2 epitope (CL-3, CL-4). While the antibody constructs of the present invention display EC₅₀ values in the two-digit picomolar range, the comparative constructs display EC₅₀ values in the three-digit up to the five-digit picomolar range. None of the constructs showed any activity against target-negative cell line (data not shown).

TABLE 5
EC₅₀ values [pM] of CLDN18.2 × CD3 antibody constructs
as measured in a 48-hour cytotoxicity assay.

| Target cell | CL-1 × I2C-scFc | CL-2 × I2C-scFc | CL-3 × I2C-scFc | CL-4 × I2C-scFc |
|---|---|---|---|---|
| GSU | 7.4 ± 0.3 | 12.1 ± 0.7 | 360.3 ± 47.2 | 1655.5 ± 71.5 |
| IM95 | 16.0 ± 2.7 | 21.4 ± 4.2 | 623.1 ± 93.3 | 5900.0 ± 514.0 |
| NUGC4 | 117.1 ± 46.2 | 97.1 ± 35.1 | 1196.5 ± 426.4 | >30000 |
| SNU-601 | 61.9 ± 6.9 | 64.7 ± 6.7 | 13797.5 ± 4169.5 | >30000 |
| SNU-620 | 29.0 ± 3.6 | 31.0 ± 5.3 | 557.1 ± 98.4 | 11093.5 ± 1996.5 |
| CHO-CLDN18.2 | 13.6 ± 0.1 | 13.2 ± 0.5 | 440.2 ± 112.5 | 3181.0 ± 119.0 |

In order to exclude that these observations were due to a significantly higher affinity of the antibody constructs of the present invention (here: CL-1, CL-2) as compared to the control constructs (here: CL-3), a cell-based affinity assay was carried out with CHO cells transfected with hu Cldn18.2. It was shown that the affinities of the three tested constructs CL-1, CL-2 and CL-3 were in a very similar range. This means that the favorable epitope/activity relationship demonstrated for the antibody constructs of the present invention is not due to the control constructs merely having a lower affinity and hence exhibiting a lower cytotoxic activity. Instead, the potency seems to be due to the particular epitope within CLDN18.2 that is recognized by the present antibody constructs.

### Example 7.5

### Potency of Redirecting Macaque T Cells Against CLDN18.2-Expressing CHO Cells

The cytotoxic activity of CLDN18.2×CD3 antibody constructs was analyzed in a 48 h FACS-based cytotoxicity assay using CHO cells transfected with human CLDN18.2 as target cells, and the macaque T cell line 4119LnPx (Knappe et al. Blood 95:3256-61 (2000)) as source of effector cells at an E:T ratio of 10:1. Note that human and cynomolgus CLDN18.2 share the identical amino acid sequence in the extracellular domains. Target cell labeling of transfected CHO cells and flow cytometry based analysis of cytotoxic activity was performed as described in Example 7.2 above.

Results are shown in Table 6. Macaque T cells from cell line 4119LnPx were induced to efficiently kill CLDN18.2-transfected CHO cells by CLDN18.2×CD3 antibody constructs according to the invention. The antibody constructs presented potently with 1-digit to 2-digit picomolar EC50-values in this assay, confirming that they are very active in the macaque system.

TABLE 6
EC50 values [pM] of CLDN18.2 × CD3 antibody constructs
as measured in a 48-hour FACS-based cytotoxicity assay with
macaque T cell line 4119LnPx as effector cells and CHO cells
transfected with human CLDN18.2 as target cells.

| CLDN18.2 × CD3 antibody construct | EC50 [pM] |
|---|---|
| CL-1 × I2C-scFc | 38 |
| CL-1 × I2C-6His | 7.2 |

### Example 8

### Monomer to Dimer Conversion after (i) Three Freeze/Thaw Cycles and (ii) 7 Days of Incubation at 37° C.

CLDN18.2×CD3 monomeric antibody constructs were subjected to different stress conditions followed by high performance SEC to determine the percentage of initially monomeric antibody construct which had been converted into dimeric antibody construct.

(i) 25 μg of monomeric antibody construct were adjusted to a concentration of 250 μg/ml with generic formulation buffer and then frozen at −80ºC for 30 min followed by thawing for 30 min at room temperature. After three freeze/thaw cycles the dimer content was determined by HP-SEC.

(ii) 25 μg of monomeric antibody construct were adjusted to a concentration of 250 μg/ml with generic formulation buffer followed by incubation at 37ºC for 7 days. The dimer content was determined by HP-SEC.

A high-performance (HP) silica-based size exclusion liquid chromatography (SEC) column was connected to an FPLC equipped with an autosampler. Column equilibration and running buffer consisted of 100 mM KH₂PO₄—200 mM Na₂SO₄ adjusted to pH 6.6. The antibody construct solution (25 μg protein) was applied to the equilibrated column and elution was carried out at a flow rate of 0.75 ml/min at a maximum pressure of 7 MPa. The whole run was monitored at 280, 254 and 210 nm optical absorbance. Analysis was done by peak integration of the 210 nm signal recorded in the software run evaluation sheet. Dimer content was calculated by dividing the area of the dimer peak by the total area of monomer plus dimer peak.

The results are shown in Table 7 below. The analyzed CLDN18.2×CD3 antibody constructs presented with dimer percentages of 0.0% after three freeze/thaw cycles, and with dimer percentages of ≤2% after 7 days of incubation at 37° C.

TABLE 7
Percentage of monomeric versus dimeric CLDN18.2 ×
CD3 antibody constructs as determined by High Performance
Size Exclusion Chromatography (HP-SEC).

| | Percentage of dimer | Percentage of dimer |
|---|---|---|
| CLDN18.2 × CD3 | after three freeze/ | after 7 days of |
| antibody construct | thaw cycles | incubation at 37° C. |
| CL-1 × I2C-scFc | 0.0 | 0.0 |
| CL-1 × I2C-6His | 0.72 | 0.0 |

### Example 9

### Thermostability

Antibody aggregation temperature was determined as follows: 40 μl of antibody construct solution at a concentration of 250 μg/ml are transferred into a single use cuvette and placed in a dynamic light scattering device. The sample was heated from 40° ° C. to 70° C. at a heating rate of 0.5° C./min with constant acquisition of the measured radius. Increase of radius indicating melting of the protein and aggregation was used by the software package delivered with the DLS device to calculate the aggregation temperature of the antibody construct.

The antibody construct CL-1×I2C-scFc was shown to have a beneficial aggregation temperature of ≥51° C., more specifically, of 51.7° ° C. In a separate assay, the aggregation temperature of the molecule denominated CL-1×I2C-6His was shown to have a value of ≥45° C., more specifically, of 49.7° C.

### Example 10

Turbidity at a concentration of the monomeric antibody construct of 2.5 mg/ml

1 ml of purified antibody construct solution of a concentration of 250 μg/ml was concentrated by spin concentration units to 2500 μg/ml. After 16h storage at 5° C. the turbidity of the solution was determined by OD340 nm optical absorption measurement against the generic formulation buffer.

The antibody construct CL-1×I2C-scFc was shown to have a very favourable turbidity of ≤0.021, while CL-1×I2C-6His was shown to have a very favourable turbidity of ≤0.029. A similar measurement of turbidity after three freeze/thaw cycles resulted in a turbidity of 0.021 for CL-1×12C-scFc.

### Example 11

### Protein Homogeneity by High Resolution Cation Exchange Chromatography

The protein homogeneity of the antibody constructs of the invention was analyzed by high resolution cation exchange chromatography (CIEX).

In one assay, 50 μg of antibody construct monomer were diluted with 50 ml binding buffer A (20 mM sodium dihydrogen phosphate, 30 mM NaCl, 0.01% sodium octanate, pH 5.5), and 40 ml of this solution were applied to a 1 ml BioPro SP-F column (YMC, Germany) connected to an FPLC device. After sample binding, a wash step with further binding buffer was carried out. For protein elution, a linear increasing salt gradient using buffer B (20 mM sodium dihydrogen phosphate, 1000 mM NaCl, 0.01% sodium octanate, pH 5.5) up to 50% percent buffer B was applied over 10 column volumes. The whole run was monitored at 280, 254 and 210 nm optical absorbance. Analysis was done by peak integration of the 280 nm signal recorded in the software run evaluation sheet. In this assay, the homogeneity of the molecule denominated “CL-1×I2C-6His” (SEQ ID NO: 131) was shown to have a value of 82.5%.

In another assay, a Bio SCX analytical CIEX column (Agilent, Frankfurt, Germany) was connected to a UPLC device (Waters, Eschborn, Germany) and equilibrated with buffer A (binding buffer) consisting of 50 mM MES, pH 5.6, 0.05% sodium azide. 10 μg of antibody construct monomer was applied to the column and bound to the column matrix. After sample binding, a wash step with further binding buffer B was carried out. For protein elution, a linear increasing salt gradient using buffer B consisting of 50 mM MES, 1000 mM sodium chloride, pH 5.6, 0.05% sodium azide up to 50% percent buffer B was applied. The whole run was monitored at 280 nm optical absorbance. Analysis was done by peak integration of the 280 nm signal recorded in the software run evaluation sheet. In this assay, the molecule denominated “CL-1×I2C-scFc”(SEQ ID NO: 132) was shown to have a homogeneity of 97,6% (area under the curve (=AUC) of the main peak).

### Example 12

### Surface Hydrophobicity as Measured by HIC Butyl

The surface hydrophobicity of antibody constructs CL-1×I2C-scFc and CL-1×I2C-6His was tested in Hydrophobic Interaction Chromatography HIC in flow-through mode.

50 μg of monomeric antibody construct were diluted with generic formulation buffer to a final volume of 500 μl (10 mM citric acid, 75 mM lysine HCl, 4% trehalose, pH 7.0) and applied to a 1 ml Butyl Sepharose FF column connected to a Äkta Purifier FPLC system. The whole run was monitored at 280, 254 and 210 nm optical absorbance. Analysis was done by peak integration of the 280 nm signal recorded in the Äkta Unicorn software run evaluation sheet. Elution behavior was evaluated by comparing area and velocity of rise and decline of protein signal thereby indicating the strength of interaction of the antibody construct with the matrix.

The antibody construct had a good elution behaviour, which was rapid and complete.

### Example 13

### Efficacy Evaluation of a CLDN18.2×CD3 Antibody Construct in a GSU-Luc Advanced Stage Gastric Cancer Model in NOD/SCID Mice

The anti-tumor activity of the CLDN18.2×CD3 antibody construct having SEQ ID NO: 132 (CL-1×I2C-scFc) was tested in a model of female NOD/SCID mice which were subcutaneously injected on day 1 with 5×10⁶ GSU-luc (luciferase) cells. 2×10⁷ effector cells (in vitro expanded and activated human CD3⁺ T cells) were injected intraperitoneally on day 8. Treatment occurred on days 12, 19 and 26 (Q7D×3). Two control groups, one w/o T cells (group 1), another one with T cells (group 2) were treated with 0.1 ml/admin of vehicle (25 mM L-lysine, 0.002% (w/v) polysorbate 80 in 0.9% (w/v) sodium chloride pH 7.0) by intravenous bolus injections. The antibody construct was administered at a concentration of 1 mg/kg/admin by intravenous bolus injections in a final volume of 0.1 ml (group 3). The number of mice per group was 5 (group 1), 10 (group 2) and 10 (group 3).

Tumors were measured by caliper during the study and progress evaluated by intergroup comparison of tumor volumes (TV). The tumor growth inhibition T/C [%] on day x is determined by calculating the tumor volume as T/C (%)=100×(median TV of analyzed group)/(median TV of control group), and the calculated values are depicted in Table 8.

TABLE 8
T/C [%] values of the GSU-luc advanced stage gastric cancer
model in NOD/SCID mice.

| | Group 2 | | | Group 3 | | |
|---|---|---|---|---|---|---|
| Day of Study | median | n | T/C [%] | median | n | T/C [%] |
| 11 | 208 | 10 | 100 | 208 | 10 | 100 |
| 13 | 275 | 10 | 100 | 316 | 10 | 115 |
| 15 | 430 | 10 | 100 | 369 | 10 | 86 |
| 18 | 695 | 10 | 100 | 185 | 10 | 27 |
| 20 | 891 | 10 | 100 | 123 | 10 | 14 |
| 22 | 896 | 4 | 100 | 79 | 10 | 9 |
| 25 | 1373 | 2 | 100 | 52 | 9 | 4 |

“Median” = median tumor volume of analyzed group.

The results are furthermore shown in FIG. 8. Significant tumor growth inhibition was shown in the NOD/SCID mouse model in the present advanced stage tumor analysis. Furthermore, treatment with the CLDN18.2×CD3 antibody construct had no effect on the body weight of the mice (data not shown).

### Example 14

### Evaluation of the Anti-Tumor Activity of a CLDN18.2×CD3 Antibody Construct in a SNU620-Luc Tumor Formation Xenograft Model in Female Athymic Nude Mice

The anti-tumor activity of the CLDN18.2×CD3 antibody construct having SEQ ID NO: 132 (CL-1×I2C-scFc) was tested in a model of female athymic nude mice which were subcutaneously injected on day 1 with SNU620-luc (5×10⁶ target cells)/PBMC (2.5×10⁶ effector cells) mix in 50% matrigel. Treatment of groups 1-4 occurred on days 3, 10 and 17 (Q7D). The control group which received tumor/PBMC mix (group 1) was treated with vehicle (25 mM L-lysine, 0.002% (w/v) polysorbate 80 in 0.9% (w/v) sodium chloride pH 7.0) at 0.1 ml/admin by intravenous bolus injections. The antibody construct was administered at a concentration of 1 mg/kg/admin (group 2), 0.1 mg/kg/admin (group 3) and 0.01 mg/kg/admin (group 4) in intravenous bolus injections at a volume of 0.1 ml. The number of mice per group was 10.

Tumors were measured by caliper during the study and progress evaluated by intergroup comparison of tumor volumes (TV). The tumor growth inhibition T/C [%] on day x is determined by calculating the tumor volume as T/C (%)=100×(median TV of analyzed group)/(median TV of control group), and the calculated values are depicted in Table 9.

TABLE 9
T/C [%] values of the SNU620-luc tumor formation xenograft model
in female athymic nude mice.

| | Group 1 | | Group 2 | | Group 3 | | Group 4 | | Group 5 | |
|---|---|---|---|---|---|---|---|---|---|---|
| Day | median | T/C [%] | median | T/C [%] | median | T/C [%] | median | T/C [%] | median | T/C [%] |
| 5 | 142 | 100 | 97 | 68 | 107 | 76 | 148 | 104 | 156 | 110 |
| 8 | 196 | 100 | 53 | 27 | 96 | 49 | 110 | 56 | 180 | 91 |
| 10 | 231 | 100 | 34 | 15 | 51 | 22 | 83 | 36 | 217 | 94 |
| 12 | 274 | 100 | 21 | 8 | 29 | 11 | 46 | 17 | 168 | 61 |
| 15 | 321 | 100 | 13 | 4 | 19 | 6 | 27 | 8 | 70 | 22 |
| 17 | 353 | 100 | 10 | 3 | 13 | 4 | 21 | 6 | 44 | 12 |
| 19 | 448 | 100 | 8 | 2 | 11 | 2 | 16 | 4 | 53 | 12 |
| 22 | 552 | 100 | 7 | 1 | 10 | 2 | 15 | 3 | 64 | 12 |

“Median” = median tumor volume of analyzed group.

The results are furthermore shown in FIG. 9. Significant tumor growth inhibition was shown in the athymic nude mouse model with all tested concentrations of the CLDN18.2×CD3 antibody construct. Furthermore, treatment with the CLDN18.2×CD3 antibody construct had no effect on the body weight of the mice (data not shown).

### Example 15

### Cyno Exploratory Toxicology Study

An exploratory NHP tolerability study was carried out with a CLDN18.2×CD3 antibody construct CL-1×I2C-6His (SEQ ID NO: 131) by intravenous administration. Systemic exposures achieved after the starting dose of 25 μg/kg/day (˜20× the human EC90) were well tolerated. Dose escalation to 125 μg/kg/day (˜120× the human EC90) resulted in clinical as well as histopathological effects expected from a T cell engaging antibody construct targeting CLDN18.2

### Example 16

### In Vitro Combination Therapy Studies

Treatment with CL-1×I2C-scFc activates human T cells, leading to upregulation of PD-1 on T cells (data not shown). The treatment can also lead to upregulation of PD-L1 on tumor cells. GSU and NUG-C4 gastric cancer cell lines which are CLDN18.2 positive were engineered to overexpress PD-L1. Cells were incubated with CL-1×I2C-scFc, and with activated human T cells in the absence or presence of an anti-PD-1 antibody having a heavy chain amino acid sequence as depicted in SEQ ID NO: 360 and a light chain amino acid sequence as depicted in SEQ ID NO: 361. Cytotoxicity was assessed after a 24 h incubation. The results are shown in Table 10 below.

TABLE 10
EC₅₀ and EC₉₀ values [pM] of two separate cytotoxicity
assays for two different cell lines each

| | GSU | | NUG-C4 | |
|---|---|---|---|---|
| | EC₅₀ | EC₉₀ | EC₅₀ | EC₉₀ |
| CL-1 × I2C-scFc | 52.8 ± 9.4 | 168.0 ± 77.7 | 168.0 ± 85.1 | 712 ± 91.7 |
| alone | | | | |
| CL-1 × I2C-scFc + | 28.1 ± 10.7 | 83.4 ± 45.0 | 63.9 ± 57.0 | 219.9 ± 77.2 |
| anti-PD-1 ab | | | | |

This assay demonstrates that the addition of an anti-PD-1 antibody increases the efficacy of the CD3×CLDN18.2 antibody constructs of the invention.

### Example 17

### Combination Therapy Studies in Mice

Using a human/murine chimeric CD3 epsilon knock-in mouse model, therapeutic combinations of antibodies/antibody constructs were evaluated to potentially enhance efficacy of bispecific antibody constructs as described herein. A bispecific single-chain anti-human CD3 (“I2C” scFv)×anti-mouse CLDN18.2 (scFv)-scFc surrogate antibody construct was generated. This molecule demonstrated potent activity against mouse CLDN18.2-expressing cells in vitro. The molecule was then tested in a genetically modified immune-competent mouse model (human/murine chimeric CD3 epsilon knock-in) for its anti-tumor activity in combination with anti-mouse PD-1, anti-mouse CTLA4 and anti-mouse 4-1BB (CD137) antibodies against CLDN18.2-positive sub-cutaneously implanted tumors (B16F10 muCLDN18.2 syngeneic model). According to the study design, the mice were randomized on day 10, having a tumor volume of about 50-100 mm³. Ten mice were included in each group. The CD3×CLDN18.2 antibody construct (150 μg/kg) or a control (anti-CD3×anti-EGFRvIII, 150 μg/kg) bispecific antibody construct were administered (i.v.) on days 11 and 18, and the antibodies (anti-PD-1 100 μg; anti-4-1BB 150 μg; anti-CTLA4 300 μg; antibody idotype as control) were dosed on days 11, 14, 17 and 20. Tumor volumes were measured on days 10, 13, 17, 20 and 25 (terminal harvest, all groups).

All three antibodies enhanced efficacy of the bispecific construct. The agonistic anti-4-1BB monoclonal antibody demonstrated no single-agent activity against the CLDN18.2 positive tumors. Specifically, tumor growth inhibition (TGI) on day 25 was determined as follows:

| Control bispecific antibody construct + antibody isotype | → 0% (standard) |
|---|---|
| Control bispecific antibody construct + anti-4-1BB ab | → not significant |
| Control bispecific antibody construct + anti-CTLA-4 ab | → 21% |
| CD3 × CLDN18.2 antibody construct + antibody isotype | → 36% |
| Control bispecific antibody construct + anti-PD-1 ab | → 57% |
| CD3 × CLDN18.2 antibody construct + anti-CTLA-4 ab | → 76% |
| CD3 × CLDN18.2 antibody construct + anti-4-1BB ab | → 77% |
| CD3 × CLDN18.2 antibody construct + anti-PD-1 ab | → 79% |

1-19. (canceled)

20. A method of inducing cytotoxicity in a cell expressing Claudin 18.2 (CLDN18.2) comprising:
contacting the cell with a bispecific antibody construct comprising a first domain which binds to human CLDN18.2, and a second domain which binds to human CD3, wherein the bispecific antibody construct has a half maximal effective concentration (EC₅₀) of ≤300 pM as measured in cell-based viability assay comprising combining CLDN18.2-positive human cells and human T cells at a ratio of about 1:10, respectively,
incubating the combined cells with the bispecific antibody construct in a culture medium for about 48 hours at about 37ºC, and
performing an assay to measure cell viability.

21. The method of claim 20, wherein the antibody construct is a human or humanized antibody.

22. The method of claim 20, wherein the antibody construct is in a format of (scFv)2.

23. The method of 20, wherein the antibody construct is a single chain antibody construct.

24. The method of claim 20, wherein the antibody construct further comprises a half-life extending domain.

25. The method of claim 24, wherein the half-life extending domain is an Fc constant domain.

26. The method of claim 20, wherein the first binding domain and the second binding domain are linked via at least one disulfide bond.

27. The method of claim 20, wherein the human CD3 is human CD3 epsilon.

28. The method of claim 20, wherein the first binding domain comprises a variable heavy chain and a variable light chain.

29. The method of claim 20, wherein the second binding domain comprises a variable heavy chain and a variable light chain.

30. The method of claim 20, wherein the cell is in a human subject.

31. The method of claim 30, wherein the antibody construct is delivered to the subject by infusion or injection.

32. The method of claim 30, wherein the antibody construct is delivered to the subject intravenously, intramuscularly, or subcutaneously.

33. The method of claim 30, wherein the subject suffers from a CLDN18.2-expressing disease or neoplasm.

34. The method of claim 33, wherein the disease or neoplasm is gastrointestinal cancer, ovarian cancer, or lung cancer.

35. The method of claim 34, wherein the gastrointestinal cancer is gastric cancer, esophageal cancer, gastroesophageal cancer, pancreatic cancer, or colorectal cancer.

36. The method of claim 20, wherein the CLDN18-2-positive human cells are GSU cells, NUGC4 cells, IM95 cells, SNU-620 cells, SNU-601 cells, or CHO-CLDN18.2 cells.

37. A method of treating a subject suffering from a Claudin 18.2 (CLDN18.2)-expressing disease or neoplasm comprising:
administering to a subject diagnosed as having the CLDN18.2-expressing disease or neoplasm an effective amount of a bispecific antibody construct comprising a first domain which binds to human CLDN18.2 and a second domain which binds to human CD3,
wherein the bispecific antibody construct has a half maximal effective concentration (EC₅₀) of ≤300 pM as measured in cell-based viability assay comprising combining CLDN18.2-positive human cells and human T cells at a ratio of about 1:10, respectively;
incubating the combined cells with the bispecific antibody construct in a culture medium for about 48 hours at about 37ºC; and
performing an assay to measure cell viability.

38. The method of claim 37, wherein the subject is a human subject.

39. The method of claim 37, wherein the administering is by infusion or injection.

40. The method of claim 37, wherein the administering is intravenous, intramuscular, or subcutaneous.

41. The method of claim 37, wherein the subject suffers from a CLDN18.2-expressing disease or neoplasm.

42. The method of claim 41, wherein the disease or neoplasm is gastrointestinal cancer, ovarian cancer, or lung cancer.

43. The method of claim 41, wherein the gastrointestinal cancer is gastric cancer, esophageal cancer, gastroesophageal cancer, pancreatic cancer, or colorectal cancer.


SEQUENCE LISTING (371 sequences)

SEQ ID NO: 1 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 2 - protein, 53 aa, Homo sapiens
DQWSTQDLYNNPVTAVFNYQGLWRSCVRESSGFTECRGYFTLLGLPAMLQAVR

SEQ ID NO: 3 - protein, 31 aa, Homo sapiens
VTNFWMSTANMYTGMGGMVQTVQTRYTFGAA

SEQ ID NO: 4 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 5 - protein, 211 aa, Homo sapiens
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYV

SEQ ID NO: 6 - protein, 230 aa, Homo sapiens
MASLGLQLVGYILGLLGLLGTLVAMLLPSWKTSSYVGASIVTAVGFSKGLWMECATHSTGITQCDIYSTLLGLPADIQAAQAMMVTSSAISSLACIISVVGMRCTVFCQESRAKDRVAVAGGVFFILGGLLGFIPVAWNLHGILRDFYSPLVPDSMKFEIGEALYLGIISSLFSLIAGIILCFSCSSQRNRSNYYDAYQAQPLATRSSPRPGQPPKVKSEFNSYSLTGYV

SEQ ID NO: 7 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 8 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 9 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 10 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 11 - protein, 261 aa, synthetic construct
MAVTACQGLGFVVSLIGIAGIIAATCMDMWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 12 - protein, 261 aa, synthetic construct
MAVTACQGLGFVVSLIGIAGIIAATCMDQWSTQDLYDNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 13 - protein, 261 aa, synthetic construct
MAVTACQGLGFVVSLIGIAGIIAATCMDMWSTQDLYDNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 14 - protein, 261 aa, synthetic construct
MAVTACQGLGFVVSLIGIAGIIAATCMDQWSTQDLYNNPVTSVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 15 - protein, 261 aa, synthetic construct
MAVTACQGLGFVVSLIGIAGIIAATCMDQWSTQDLYNNPVTAVFQYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 16 - protein, 261 aa, synthetic construct
MAVTACQGLGFVVSLIGIAGIIAATCMDQWSTQDLYNNPVTAVFNYEGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 17 - protein, 261 aa, synthetic construct
MAVTACQGLGFVVSLIGIAGIIAATCMDQWSTQDLYNNPVTSVFQYEGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 18 - protein, 261 aa, synthetic construct
MAVTACQGLGFVVSLIGIAGIIAATCMDQWSTQDLYNNPVTAVFNYQGLWRSCVRQSSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 19 - protein, 261 aa, synthetic construct
MAVTACQGLGFVVSLIGIAGIIAATCMDQWSTQDLYNNPVTAVFNYQGLWRSCVRESSGFTECRPYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 20 - protein, 261 aa, synthetic construct
MAVTACQGLGFVVSLIGIAGIIAATCMDQWSTQDLYNNPVTAVFNYQGLWRSCVRESSGFTECRGYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 21 - protein, 261 aa, synthetic construct
MAVTACQGLGFVVSLIGIAGIIAATCMDQWSTQDLYNNPVTAVFNYQGLWRSCVRESSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 22 - protein, 261 aa, synthetic construct
MAVTACQGLGFVVSLIGIAGIIAATCMDQWSTQDLYNNPVTAQVVWEGLWMSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 23 - protein, 250 aa, synthetic construct
MAVTACQGLGFVVSLIGIAGIIAATCMDQWSTQDLYNNPVTAVFNYQGLWRSCVVQSTGQMQCKVYFTLLGLPAMLQAVRALMIVGIVLGAIGLLVSIFALKCIRIGSMEDSAKANMTLTSGIMFIVSGLCAIAGVSVFANMLIQDFYNPLVAEALKRELGASLFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 24 - protein, 261 aa, synthetic construct
MAVTACQGLGFVVSLIGIAGIIAATCMDQWSTQDLYNNPVTAVFNYQGLWRSCVRESSGFTECRGYDSLLALPQDLQAARALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 25 - protein, 250 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLIQDFYNPLVAEALKRELGASLFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 26 - protein, 251 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLIRDFYGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 27 - protein, 260 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMNPLVAEAQKRELGASLFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 28 - protein, 14 aa, synthetic construct
GSSTGAVTSGYYPN

SEQ ID NO: 29 - protein, 7 aa, synthetic construct
GTKFLAP

SEQ ID NO: 30 - protein, 9 aa, synthetic construct
ALWYSNRWV

SEQ ID NO: 31 - protein, 5 aa, synthetic construct
IYAMN

SEQ ID NO: 32 - protein, 19 aa, synthetic construct
RIRSKYNNYATYYADSVKS

SEQ ID NO: 33 - protein, 14 aa, synthetic construct
HGNFGNSYVSFFAY

SEQ ID NO: 34 - protein, 125 aa, synthetic construct
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 35 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 36 - protein, 249 aa, synthetic construct
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 37 - protein, 14 aa, synthetic construct
GSSTGAVTSGYYPN

SEQ ID NO: 38 - protein, 7 aa, synthetic construct
GTKFLAP

SEQ ID NO: 39 - protein, 9 aa, synthetic construct
ALWYSNRWV

SEQ ID NO: 40 - protein, 5 aa, synthetic construct
KYAMN

SEQ ID NO: 41 - protein, 19 aa, synthetic construct
RIRSKYNNYATYYADSVKD

SEQ ID NO: 42 - protein, 14 aa, synthetic construct
HGNFGNSYISYWAY

SEQ ID NO: 43 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 44 - protein, 109 aa, synthetic construct; identical to SEQ ID NO: 35

SEQ ID NO: 45 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 46 - protein, 14 aa, synthetic construct
GSSTGAVTSGYYPN

SEQ ID NO: 47 - protein, 7 aa, synthetic construct
GTKFLAP

SEQ ID NO: 48 - protein, 9 aa, synthetic construct
ALWYSNRWV

SEQ ID NO: 49 - protein, 5 aa, synthetic construct
SYAMN

SEQ ID NO: 50 - protein, 19 aa, synthetic construct
RIRSKYNNYATYYADSVKG

SEQ ID NO: 51 - protein, 14 aa, synthetic construct
HGNFGNSYLSFWAY

SEQ ID NO: 52 - protein, 125 aa, synthetic construct
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 53 - protein, 109 aa, synthetic construct; identical to SEQ ID NO: 35

SEQ ID NO: 54 - protein, 249 aa, synthetic construct
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 55 - protein, 14 aa, synthetic construct
GSSTGAVTSGYYPN

SEQ ID NO: 56 - protein, 7 aa, synthetic construct
GTKFLAP

SEQ ID NO: 57 - protein, 9 aa, synthetic construct
ALWYSNRWV

SEQ ID NO: 58 - protein, 5 aa, synthetic construct
RYAMN

SEQ ID NO: 59 - protein, 19 aa, synthetic construct
RIRSKYNNYATYYADSVKG

SEQ ID NO: 60 - protein, 14 aa, synthetic construct
HGNFGNSYLSYFAY

SEQ ID NO: 61 - protein, 125 aa, synthetic construct
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 62 - protein, 109 aa, synthetic construct; identical to SEQ ID NO: 35

SEQ ID NO: 63 - protein, 249 aa, synthetic construct
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 64 - protein, 14 aa, synthetic construct
RSSTGAVTSGYYPN

SEQ ID NO: 65 - protein, 7 aa, synthetic construct
ATDMRPS

SEQ ID NO: 66 - protein, 9 aa, synthetic construct
ALWYSNRWV

SEQ ID NO: 67 - protein, 5 aa, synthetic construct
VYAMN

SEQ ID NO: 68 - protein, 19 aa, synthetic construct
RIRSKYNNYATYYADSVKK

SEQ ID NO: 69 - protein, 14 aa, synthetic construct
HGNFGNSYLSWWAY

SEQ ID NO: 70 - protein, 125 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 71 - protein, 109 aa, synthetic construct
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 72 - protein, 249 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 73 - protein, 14 aa, synthetic construct
GSSTGAVTSGYYPN

SEQ ID NO: 74 - protein, 7 aa, synthetic construct
GTKFLAP

SEQ ID NO: 75 - protein, 9 aa, synthetic construct
ALWYSNRWV

SEQ ID NO: 76 - protein, 5 aa, synthetic construct
KYAMN

SEQ ID NO: 77 - protein, 19 aa, synthetic construct
RIRSKYNNYATYYADSVKS

SEQ ID NO: 78 - protein, 14 aa, synthetic construct
HGNFGNSYTSYYAY

SEQ ID NO: 79 - protein, 125 aa, synthetic construct
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 80 - protein, 109 aa, synthetic construct; identical to SEQ ID NO: 35

SEQ ID NO: 81 - protein, 249 aa, synthetic construct
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 82 - protein, 14 aa, synthetic construct
RSSTGAVTSGYYPN

SEQ ID NO: 83 - protein, 7 aa, synthetic construct
ATDMRPS

SEQ ID NO: 84 - protein, 9 aa, synthetic construct
ALWYSNRWV

SEQ ID NO: 85 - protein, 5 aa, synthetic construct
GYAMN

SEQ ID NO: 86 - protein, 19 aa, synthetic construct
RIRSKYNNYATYYADSVKE

SEQ ID NO: 87 - protein, 14 aa, synthetic construct
HRNFGNSYLSWFAY

SEQ ID NO: 88 - protein, 125 aa, synthetic construct
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 89 - protein, 109 aa, synthetic construct; identical to SEQ ID NO: 71

SEQ ID NO: 90 - protein, 249 aa, synthetic construct
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 91 - protein, 14 aa, synthetic construct
GSSTGAVTSGYYPN

SEQ ID NO: 92 - protein, 7 aa, synthetic construct
GTKFLAP

SEQ ID NO: 93 - protein, 9 aa, synthetic construct
ALWYSNRWV

SEQ ID NO: 94 - protein, 5 aa, synthetic construct
VYAMN

SEQ ID NO: 95 - protein, 19 aa, synthetic construct
RIRSKYNNYATYYADSVKK

SEQ ID NO: 96 - protein, 14 aa, synthetic construct
HGNFGNSYISWWAY

SEQ ID NO: 97 - protein, 125 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 98 - protein, 109 aa, synthetic construct; identical to SEQ ID NO: 35

SEQ ID NO: 99 - protein, 249 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 100 - protein, 14 aa, synthetic construct
GSSTGAVTSGNYPN

SEQ ID NO: 101 - protein, 7 aa, synthetic construct
GTKFLAP

SEQ ID NO: 102 - protein, 9 aa, synthetic construct
VLWYSNRWV

SEQ ID NO: 103 - protein, 5 aa, synthetic construct
SYAMN

SEQ ID NO: 104 - protein, 19 aa, synthetic construct
RIRSKYNNYATYYADSVKG

SEQ ID NO: 105 - protein, 14 aa, synthetic construct
HGNFGNSYVSWWAY

SEQ ID NO: 106 - protein, 125 aa, synthetic construct
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 107 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 108 - protein, 249 aa, synthetic construct
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 109 - protein, 14 aa, synthetic construct
GSSTGAVTSGNYPN

SEQ ID NO: 110 - protein, 7 aa, synthetic construct
GTKFLAP

SEQ ID NO: 111 - protein, 9 aa, synthetic construct
VLWYSNRWV

SEQ ID NO: 112 - protein, 5 aa, synthetic construct
KYAMN

SEQ ID NO: 113 - protein, 19 aa, synthetic construct
RIRSKYNNYATYYADSVKD

SEQ ID NO: 114 - protein, 14 aa, synthetic construct
HGNFGNSYISYWAY

SEQ ID NO: 115 - protein, 125 aa, synthetic construct; identical to SEQ ID NO: 43

SEQ ID NO: 116 - protein, 109 aa, synthetic construct; identical to SEQ ID NO: 107

SEQ ID NO: 117 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 118 - protein, 125 aa, synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 119 - protein, 109 aa, synthetic construct; identical to SEQ ID NO: 107

SEQ ID NO: 120 - protein, 249 aa, synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 121 - protein, 5 aa, synthetic construct
GYYMH

SEQ ID NO: 122 - protein, 17 aa, synthetic construct
WINPNSGGTKYAQKFQG

SEQ ID NO: 123 - protein, 16 aa, synthetic construct
DRITVAGTYYYYGMDV

SEQ ID NO: 124 - protein, 11 aa, synthetic construct
RASQGVNNWLA

SEQ ID NO: 125 - protein, 7 aa, synthetic construct
TASSLQS

SEQ ID NO: 126 - protein, 9 aa, synthetic construct
QQANSFPIT

SEQ ID NO: 127 - protein, 125 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 128 - protein, 107 aa, synthetic construct
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 129 - protein, 247 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 130 - protein, 502 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 131 - protein, 508 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 132 - protein, 990 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 5 aa, synthetic construct
GYYMH

SEQ ID NO: 134 - protein, 17 aa, synthetic construct
WINPNSGGTKYAQKFQG

SEQ ID NO: 135 - protein, 16 aa, synthetic construct
DRITVAGTYYYYGMDV

SEQ ID NO: 136 - protein, 11 aa, synthetic construct
RASQGVNNWLA

SEQ ID NO: 137 - protein, 7 aa, synthetic construct
TASSLQS

SEQ ID NO: 138 - protein, 9 aa, synthetic construct
QQANSFPIT

SEQ ID NO: 139 - protein, 125 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 140 - protein, 107 aa, synthetic construct; identical to SEQ ID NO: 128

SEQ ID NO: 141 - protein, 247 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 142 - protein, 502 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 143 - protein, 508 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 144 - protein, 990 aa, synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 118 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 146 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 147 - protein, 246 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 148 - protein, 501 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 149 - protein, 989 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 118 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 151 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 152 - protein, 246 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 153 - protein, 501 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 154 - protein, 989 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 4 aa, synthetic construct
GGGG

SEQ ID NO: 156 - protein, 5 aa, synthetic construct
GGGGS

SEQ ID NO: 157 - protein, 5 aa, synthetic construct
GGGGQ

SEQ ID NO: 158 - protein, 6 aa, synthetic construct
SGGGGS

SEQ ID NO: 159 - protein, 6 aa, synthetic construct
PGGGGS

SEQ ID NO: 160 - protein, 6 aa, synthetic construct
PGGDGS

SEQ ID NO: 161 - protein, 9 aa, synthetic construct
GGGGSGGGS

SEQ ID NO: 162 - protein, 10 aa, synthetic construct
GGGGSGGGGS

SEQ ID NO: 163 - protein, 15 aa, synthetic construct
GGGGSGGGGSGGGGS

SEQ ID NO: 164 - protein, 20 aa, synthetic construct
GGGGSGGGGSGGGGSGGGGS

SEQ ID NO: 165 - protein, 25 aa, synthetic construct
GGGGSGGGGSGGGGSGGGGSGGGGS

SEQ ID NO: 166 - protein, 30 aa, synthetic construct
GGGGSGGGGSGGGGSGGGGSGGGGSGGGGS

SEQ ID NO: 167 - protein, 35 aa, synthetic construct
GGGGSGGGGSGGGGSGGGGSGGGGSGGGGSGGGGS

SEQ ID NO: 168 - protein, 40 aa, synthetic construct
GGGGSGGGGSGGGGSGGGGSGGGGSGGGGSGGGGSGGGGS

SEQ ID NO: 169 - protein, 6 aa, synthetic construct
HHHHHH

SEQ ID NO: 170 - protein, 16 aa, synthetic construct
RDWDFDVFGGGTPVGG

SEQ ID NO: 171 - protein, 16 aa, synthetic construct
  VARIANT 12: note = X can be any naturally occurring amino acid
QRFVTGHFGGLXPANG

SEQ ID NO: 172 - protein, 16 aa, synthetic construct
QRFVTGHFGGLYPANG

SEQ ID NO: 173 - protein, 16 aa, synthetic construct
QRFVTGHFGGLHPANG

SEQ ID NO: 174 - protein, 9 aa, synthetic construct
TGHFGGLHP

SEQ ID NO: 175 - protein, 16 aa, synthetic construct
QRFCTGHFGGLHPCNG

SEQ ID NO: 176 - protein, 585 aa, Homo sapiens [sequence omitted]

SEQ ID NO: 177 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 178 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 179 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 180 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 181 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 182 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 183 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 184 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 185 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 186 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 187 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 188 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 189 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 190 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 191 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 192 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 193 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 194 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 195 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 196 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 197 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 198 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 199 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 200 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 201 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 202 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 203 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 204 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 205 - protein, 585 aa, synthetic construct
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 206 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 207 - protein, 333 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 208 - protein, 330 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 209 - protein, 338 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 210 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 211 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 212 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 213 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 214 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 215 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 216 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 217 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 219 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 221 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 223 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 225 - protein, 484 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 480 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 227 - protein, 484 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 480 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 229 - protein, 484 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 480 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 231 - protein, 484 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 480 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 233 - protein, 109 aa, synthetic construct
ELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 234 - protein, 109 aa, synthetic construct
ELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 235 - protein, 109 aa, synthetic construct
ELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 236 - protein, 125 aa, synthetic construct
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 237 - protein, 125 aa, synthetic construct
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 238 - protein, 125 aa, synthetic construct
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 239 - protein, 125 aa, synthetic construct
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 240 - protein, 125 aa, synthetic construct
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 241 - protein, 125 aa, synthetic construct
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 242 - protein, 125 aa, synthetic construct
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 243 - protein, 125 aa, synthetic construct
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 244 - protein, 125 aa, synthetic construct
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 245 - protein, 125 aa, synthetic construct; identical to SEQ ID NO: 237

SEQ ID NO: 246 - protein, 249 aa, synthetic construct
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 247 - protein, 249 aa, synthetic construct
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 248 - protein, 249 aa, synthetic construct
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 249 - protein, 249 aa, synthetic construct
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 250 - protein, 249 aa, synthetic construct
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 251 - protein, 249 aa, synthetic construct
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 252 - protein, 249 aa, synthetic construct
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 253 - protein, 249 aa, synthetic construct
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 254 - protein, 249 aa, synthetic construct
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 255 - protein, 249 aa, synthetic construct
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 256 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 257 - protein, 27 aa, Homo sapiens
QDGNEEMGGITQTPYKVSISGTTVILT

SEQ ID NO: 258 - protein, 96 aa, Callithrix jacchus
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 259 - protein, 27 aa, Callithrix jacchus
QDGNEEMGDTTQNPYKVSISGTTVTLT

SEQ ID NO: 260 - protein, 96 aa, Saguinus oedipus; identical to SEQ ID NO: 258

SEQ ID NO: 261 - protein, 27 aa, Saguinus oedipus
QDGNEEMGDTTQNPYKVSISGTTVTLT

SEQ ID NO: 262 - protein, 96 aa, Saimiri sciureus
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 263 - protein, 27 aa, Saimiri sciureus
QDGNEEIGDTTQNPYKVSISGTTVTLT

SEQ ID NO: 264 - protein, 15 aa, Homo sapiens
DQWSTQDLYNNPVTA

SEQ ID NO: 265 - protein, 12 aa, Homo sapiens
VFNYQGLWRSCV

SEQ ID NO: 266 - protein, 12 aa, Homo sapiens
RESSGFTECRGY

SEQ ID NO: 267 - protein, 14 aa, Homo sapiens
FTLLGLPAMLQAVR

SEQ ID NO: 268 - protein, 31 aa, Homo sapiens; identical to SEQ ID NO: 3

SEQ ID NO: 269 - protein, 5 aa, synthetic construct
HHHHH

SEQ ID NO: 270 - protein, 7 aa, synthetic construct
HHHHHHH

SEQ ID NO: 271 - protein, 8 aa, synthetic construct
HHHHHHHH

SEQ ID NO: 272 - protein, 6 aa, synthetic construct
DTYRYI

SEQ ID NO: 273 - protein, 6 aa, synthetic construct
TDFYLK

SEQ ID NO: 274 - protein, 11 aa, synthetic construct
MASMTGGQQMG

SEQ ID NO: 275 - protein, 14 aa, synthetic construct
GKPIPNPLLGLDST

SEQ ID NO: 276 - protein, 6 aa, synthetic construct
QYPALT

SEQ ID NO: 277 - protein, 10 aa, synthetic construct
SSTSSDFRDR

SEQ ID NO: 278 - protein, 7 aa, synthetic construct
DYKDDDK

SEQ ID NO: 279 - protein, 6 aa, synthetic construct
EYMPME

SEQ ID NO: 280 - protein, 6 aa, synthetic construct
EFMPME

SEQ ID NO: 281 - protein, 19 aa, synthetic construct
KDHLIHNVHKEFHAHAHNK

SEQ ID NO: 282 - protein, 8 aa, synthetic construct
QPELAPED

SEQ ID NO: 283 - protein, 11 aa, synthetic construct
KPPTPPPEPET

SEQ ID NO: 284 - protein, 11 aa, synthetic construct
CEQKLISEEDL

SEQ ID NO: 285 - protein, 9 aa, synthetic construct
NANNPDWDF

SEQ ID NO: 286 - protein, 15 aa, synthetic construct
KETAAAKFERQHMDS

SEQ ID NO: 287 - protein, 8 aa, synthetic construct
WSHPQFEK

SEQ ID NO: 288 - protein, 9 aa, synthetic construct
AWAHPQPGG

SEQ ID NO: 289 - protein, 6 aa, synthetic construct
HTTPHH

SEQ ID NO: 290 - protein, 11 aa, synthetic construct
YTDIEMNRLGK

SEQ ID NO: 291 - protein, 12 aa, synthetic construct
EDQVDPRLIDGK

SEQ ID NO: 292 - protein, 5 aa, synthetic construct
NYGMH

SEQ ID NO: 293 - protein, 17 aa, synthetic construct
LIWYDGSKKYYADSVKG

SEQ ID NO: 294 - protein, 16 aa, synthetic construct
DPSSLTGSTGYYGMDV

SEQ ID NO: 295 - protein, 11 aa, synthetic construct
SGDKLGDKYAC

SEQ ID NO: 296 - protein, 7 aa, synthetic construct
QDRKRPS

SEQ ID NO: 297 - protein, 9 aa, synthetic construct
QAWDSSTEV

SEQ ID NO: 298 - protein, 125 aa, synthetic construct
QVQLVESGGGVVQPGRSLRLSCAASGFTFSNYGMHWVRQAPGKGLEWVALIWYDGSKKYYADSVKGRFTISRDNSKNTLYLQMNSLRAEDTAAYYCARDPSSLTGSTGYYGMDVWGQGTTVTVSS

SEQ ID NO: 299 - protein, 106 aa, synthetic construct
SYELTQPPSVSVSPGQTASITCSGDKLGDKYACWYQQKPGQSPVLVIYQDRKRPSGIPERFSGSNSGNTATLTISGTQAMDEADYYCQAWDSSTEVFGGGTKLTVL

SEQ ID NO: 300 - protein, 477 aa, synthetic construct
MDMRVPAQLLGLLLLWLRGARCQVQLVESGGGVVQPGRSLRLSCAASGFTFSNYGMHWVRQAPGKGLEWVALIWYDGSKKYYADSVKGRFTISRDNSKNTLYLQMNSLRAEDTAAYYCARDPSSLTGSTGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 231 aa, synthetic construct
MAWALLLLTLLTQGTGSWASYELTQPPSVSVSPGQTASITCSGDKLGDKYACWYQQKPGQSPVLVIYQDRKRPSGIPERFSGSNSGNTATLTISGTQAMDEADYYCQAWDSSTEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 302 - protein, 5 aa, synthetic construct
SYDMS

SEQ ID NO: 303 - protein, 17 aa, synthetic construct
LISGGGSNTYYADSVKG

SEQ ID NO: 304 - protein, 11 aa, synthetic construct
PSGHYFYAMDV

SEQ ID NO: 305 - protein, 11 aa, synthetic construct
RASQGISNWLA

SEQ ID NO: 306 - protein, 7 aa, synthetic construct
AASSLQS

SEQ ID NO: 307 - protein, 9 aa, synthetic construct
QQADSFPHT

SEQ ID NO: 308 - protein, 120 aa, synthetic construct
EVQLLESGGGLVQPGGALRLSCAASGFTFSSYDMSWVRQAPGKGLEWVSLISGGGSNTYYADSVKGRFTISRDNSKNTLYLQMNSLRAEDTAVYFCASPSGHYFYAMDVWGQGTTVTVSS

SEQ ID NO: 309 - protein, 107 aa, synthetic construct
DIQMTQSPSSVSASVGDRVTITCRASQGISNWLAWYQQKPGKAPKLLIFAASSLQSGVPSRFGGSGSGTDFTFTISSLQPEDFATYYCQQADSFPHTFGGGTKVEIK

SEQ ID NO: 310 - protein, 472 aa, synthetic construct
MDMRVPAQLLGLLLLWLRGARCEVQLLESGGGLVQPGGALRLSCAASGFTFSSYDMSWVRQAPGKGLEWVSLISGGGSNTYYADSVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 236 aa, synthetic construct
MDMRVPAQLLGLLLLWLRGARCDIQMTQSPSSVSASVGDRVTITCRASQGISNWLAWYQQKPGKAPKLLIFAASSLQSGVPSRFGGSGSGTDFTFTISSLQPEDFATYYCQQADSFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 5 aa, synthetic construct
DYSMS

SEQ ID NO: 313 - protein, 17 aa, synthetic construct
GINWNGGRTRYADSVKG

SEQ ID NO: 314 - protein, 12 aa, synthetic construct
EFNNFESNWFDP

SEQ ID NO: 315 - protein, 11 aa, synthetic construct
SGDALPKKYAY

SEQ ID NO: 316 - protein, 7 aa, synthetic construct
EDSKRPS

SEQ ID NO: 317 - protein, 11 aa, synthetic construct
YSTDSSGNHRV

SEQ ID NO: 318 - protein, 121 aa, synthetic construct
EVQLVESGGSVVRPGGSLRLSCAASGFTVDDYSMSWVRQVPGKGLEWVSGINWNGGRTRYADSVKGRFTISRDSAKNSLYLQMNSLRAEDTALYYCAREFNNFESNWFDPWGQGTLVTVSS

SEQ ID NO: 319 - protein, 108 aa, synthetic construct
SYELTQPPSVSVSPGQTARITCSGDALPKKYAYWYQQKSGQAPVLVISEDSKRPSGIPERFSGSSSGTMATLTISGAQVEDEADYYCYSTDSSGNHRVFGGGTKLTVL

SEQ ID NO: 320 - protein, 473 aa, synthetic construct
MDMRVPAQLLGLLLLWLRGARCEVQLVESGGSVVRPGGSLRLSCAASGFTVDDYSMSWVRQVPGKGLEWVSGINWNGGRTRYADSVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 233 aa, synthetic construct
MAWALLLLTLLTQGTGSWASYELTQPPSVSVSPGQTARITCSGDALPKKYAYWYQQKSGQAPVLVISEDSKRPSGIPERFSGSSSGTMATLTISGAQVEDEADYYCYSTDSSGNHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 322 - protein, 5 aa, synthetic construct
SYDMS

SEQ ID NO: 323 - protein, 17 aa, synthetic construct
LISGGGSNTYYAESVKG

SEQ ID NO: 324 - protein, 11 aa, synthetic construct
PSGHYFYAMDV

SEQ ID NO: 325 - protein, 11 aa, synthetic construct
RASQGISNWLA

SEQ ID NO: 326 - protein, 7 aa, synthetic construct
AASSLQS

SEQ ID NO: 327 - protein, 9 aa, synthetic construct
QQAESFPHT

SEQ ID NO: 328 - protein, 120 aa, synthetic construct
EVQLLESGGGLVQPGGSLRLSCAASGFTFSSYDMSWVRQAPGKGLEWVSLISGGGSNTYYAESVKGRFTISRDNSKNTLYLQMNSLRAEDTAVYFCASPSGHYFYAMDVWGQGTTVTVSS

SEQ ID NO: 329 - protein, 107 aa, synthetic construct
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 330 - protein, 472 aa, synthetic construct
MDMRVPAQLLGLLLLWLRGARCEVQLLESGGGLVQPGGSLRLSCAASGFTFSSYDMSWVRQAPGKGLEWVSLISGGGSNTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 236 aa, synthetic construct
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 5 aa, synthetic construct
SYDMS

SEQ ID NO: 333 - protein, 17 aa, synthetic construct
LISGGGSQTYYAESVKG

SEQ ID NO: 334 - protein, 11 aa, synthetic construct
PSGHYFYAMDV

SEQ ID NO: 335 - protein, 11 aa, synthetic construct
RASQGISNWLA

SEQ ID NO: 336 - protein, 7 aa, synthetic construct
AASSLQS

SEQ ID NO: 337 - protein, 9 aa, synthetic construct
QQAESFPHT

SEQ ID NO: 338 - protein, 120 aa, synthetic construct
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 339 - protein, 107 aa, synthetic construct; identical to SEQ ID NO: 329

SEQ ID NO: 340 - protein, 472 aa, synthetic construct
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 236 aa, synthetic construct; identical to SEQ ID NO: 331

SEQ ID NO: 342 - protein, 5 aa, synthetic construct
NYGMH

SEQ ID NO: 343 - protein, 17 aa, synthetic construct
LIWYDASKKYYAESVKG

SEQ ID NO: 344 - protein, 16 aa, synthetic construct
DPSSLTGSTGYYGMDV

SEQ ID NO: 345 - protein, 11 aa, synthetic construct
SGDKLGDKYAS

SEQ ID NO: 346 - protein, 7 aa, synthetic construct
QDRKRPS

SEQ ID NO: 347 - protein, 9 aa, synthetic construct
QAFESSTEV

SEQ ID NO: 348 - protein, 125 aa, synthetic construct
QVQLVESGGGVVQPGRSLRLSCAASGFTFSNYGMHWVRQAPGKGLEWVALIWYDASKKYYAESVKGRFTISRDNSKNTLYLQMNSLRAEDTAAYYCARDPSSLTGSTGYYGMDVWGQGTTVTVSS

SEQ ID NO: 349 - protein, 106 aa, synthetic construct
SYELTQPPSVSVSPGQTASITCSGDKLGDKYASWYQQKPGQSPVLVIYQDRKRPSGIPERFSGSNSGNTATLTISGTQAMDEADYYCQAFESSTEVFGGGTKLTVL

SEQ ID NO: 350 - protein, 477 aa, synthetic construct
MDMRVPAQLLGLLLLWLRGARCQVQLVESGGGVVQPGRSLRLSCAASGFTFSNYGMHWVRQAPGKGLEWVALIWYDASKKYYAESVKGRFTISRDNSKNTLYLQMNSLRAEDTAAYYCARDPSSLTGSTGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 231 aa, synthetic construct
MAWALLLLTLLTQGTGSWASYELTQPPSVSVSPGQTASITCSGDKLGDKYASWYQQKPGQSPVLVIYQDRKRPSGIPERFSGSNSGNTATLTISGTQAMDEADYYCQAFESSTEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 352 - protein, 5 aa, synthetic construct
DYSMS

SEQ ID NO: 353 - protein, 17 aa, synthetic construct
GINWNGGRTRYADAVKG

SEQ ID NO: 354 - protein, 12 aa, synthetic construct
EFNNFESNWFDP

SEQ ID NO: 355 - protein, 11 aa, synthetic construct
SGDALPKKYAY

SEQ ID NO: 356 - protein, 7 aa, synthetic construct
EDAKRPS

SEQ ID NO: 357 - protein, 11 aa, synthetic construct
YSTDASGNHRV

SEQ ID NO: 358 - protein, 121 aa, synthetic construct
EVQLVESGGSVVRPGGSLRLSCAASGFTVDDYSMSWVRQVPGKGLEWVSGINWNGGRTRYADAVKGRFTISRDSAKNSLYLQMNSLRAEDTALYYCAREFNNFESNWFDPWGQGTLVTVSS

SEQ ID NO: 359 - protein, 108 aa, synthetic construct
SYELTQPPSVSVSPGQTARITCSGDALPKKYAYWYQQKPGQAPVLVISEDAKRPSGIPERFSGSSSGTMATLTISGAQVEDEADYYCYSTDASGNHRVFGGGTKLTVL

SEQ ID NO: 360 - protein, 473 aa, synthetic construct
MDMRVPAQLLGLLLLWLRGARCEVQLVESGGSVVRPGGSLRLSCAASGFTVDDYSMSWVRQVPGKGLEWVSGINWNGGRTRYADAVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 233 aa, synthetic construct
MAWALLLLTLLTQGTGSWASYELTQPPSVSVSPGQTARITCSGDALPKKYAYWYQQKPGQAPVLVISEDAKRPSGIPERFSGSSSGTMATLTISGAQVEDEADYYCYSTDASGNHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 362 - protein, 5 aa, synthetic construct
DYSMS

SEQ ID NO: 363 - protein, 17 aa, synthetic construct
GINWNAGRTRYADAVKG

SEQ ID NO: 364 - protein, 12 aa, synthetic construct
EFNNFESNWFDP

SEQ ID NO: 365 - protein, 11 aa, synthetic construct
SGDALPKKYAY

SEQ ID NO: 366 - protein, 7 aa, synthetic construct
EDAKRPS

SEQ ID NO: 367 - protein, 11 aa, synthetic construct
YSTDASGNHRV

SEQ ID NO: 368 - protein, 121 aa, synthetic construct
EVQLVESGGSVVRPGGSLRLSCAASGFTVDDYSMSWVRQVPGKGLEWVSGINWNAGRTRYADAVKGRFTISRDSAKNSLYLQMNSLRAEDTALYYCAREFNNFESNWFDPWGQGTLVTVSS

SEQ ID NO: 369 - protein, 108 aa, synthetic construct; identical to SEQ ID NO: 359

SEQ ID NO: 370 - protein, 473 aa, synthetic construct
MDMRVPAQLLGLLLLWLRGARCEVQLVESGGSVVRPGGSLRLSCAASGFTVDDYSMSWVRQVPGKGLEWVSGINWNAGRTRYADAVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 233 aa, synthetic construct; identical to SEQ ID NO: 361